SEQUENCE LISTING (59 sequences)

SEQ ID NO: 1 - DNA, 3182 bp, Saccharomyces cerevisiae
cctactgcgccaattgatgacaatacagacgatgataacaaaccgaagttatctgatgtagaaaaggattaaagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaagcggccgcataacttcgtataatgtatgctatacgaagttatctgccagtatacagctagccttgaaagtgatggaaaacattgtcatcggcacataaataaaaaaattatgaatcacgtgatcaacagcaaattatgtactcgtatatatgcaagcgcattccttatattgacactctttcattgggcatgaggctgtgtaaacataagctgtaacggtctcacggaacactgtgtagttgcattactgtcaggcagttatgttgcttaatataaaggcaaaggcatggcagaatcactttaaaacgtggccccacccgctgcaccctgtgcattttgtacgttactgcgaaatgactcaacgatgaaatgaaaaaattttgcttgaaattttgaaaaaaagatgtgcgggacgcattgttagctcattgaatacatcgtgatcgaatccaatcaatgtttaatttcatattaatacagaaactttttctcatactttcttcttcttttcattggtatattatctatatatcgtgttaattcctctttcgtcatttttagcatcgttataagagtaattaagaataactagaagagtctctctttatattcgtttattttatatatttaaccgctaaatttagtaaacaaaagaatctatcagaaatgagtgaatctccaatgttcgctgccaacggcatgccaaaggtaaatcaaggtgctgaagaagatgtcagaattttaggttacgacccattagcttctccagctctccttcaagtgcaaatcccagccacaccaacttctttggaaactgccaagagaggtagaagagaagctatagatattattaccggtaaagacgacagagttcttgtcattgtcggtccttgttccatccatgatctagaagccgctcaagaatacgctttgagattaaagaaattgtcagatgaattaaaaggtgatttatccatcattatgagagcatacttggagaagccaagaacaaccgtcggctggaaaggtctaattaatgaccctgatgttaacaacactttcaacatcaacaagggtttgcaatccgctagacaattgtttgtcaacttgacaaatatcggtttgccaattggttctgaaatgcttgataccatttctcctaaatacttggctgatttggtctccttcggtgccattggtgccagaaccaccgaatctcaactgcacagagaattggcctccggtttgtctttcccagttggtttcaagaacggtaccgatggtaccttaaatgttgctgtggatgcttgtcaagccgctgctcattctcaccatttcatgggtgttactaagcatggtgttgctgctatcaccactactaagggtaacgaacactgcttcgttattctaagaggtggtaaaaagggtaccaactacgacgctaagtccgttgcagaagctaaggctcaattgcctgccggttccaacggtctaatgattgactactctcacggtaactccaataaggatttcagaaaccaaccaaaggtcaatgacgttgtttgtgagcaaatcgctaacggtgaaaacgccattaccggtgtcatgattgaatcaaacatcaacgaaggtaaccaaggcatcccagccgaaggtaaagccggcttgaaatatggtgtttccatcactgatgcttgtataggttgggaaactactgaagacgtcttgaggaaattggctgctgctgtcagacaaagaagagaagttaacaagaaatagatgtttttttaatgatatatgtaacgtacattctttcctctaccactgccaattcggtattatttaattgtgtttagcgctatttactaattaactagaaactcaatttttaaaggcaaagctcgctgacctttcactgatttcgtggatgttatactatcagttactcttctgcaaaaaaaaattgagtcatatcgtagctttgggattatttttctctctctccacggctaattaggtgatcatgaaaaaatgaaaaattcatgagaaaagagtcagacatcgaaacatacataagttgatattcctttgatatcgacgactactcaatcaggttttaaaagaaaagaggcagctattgaagtagcagtatccagtttaggttttttaattatttacaagtaaagaaaaagagaatgccggtcgttcacgataacttcgtataatgtatgctatacgaagttatgcggccgcgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttcatggcctttataaaaaggaactatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgtactctaatctggtagaaagatgtttcacagactgtgtcaatgacttcacaacatcaaagctaaccaataaggaacaaacatgcatcatgaagtgctcagaaaagttcttgaagcatagcgaacgtgtagggcagcgtttccaagag

SEQ ID NO: 2 - DNA, 3275 bp, Artificial Sequence
  Synthetic Polynucleotide
cctactgcgccaattgatgacaatacagacgatgataacaaaccgaagttatctgatgtagaaaaggattaaagatgctaagagatagtgatgatatttcataaataatgtaattctatatatgttaattaccttttttgcgaggcatatttatggtgaagaataagttttgaccatcaaagaaggttaatgtggctgtggtttcagggtccataaagcttttcaattcatcattttttttttattcttttttttgattccggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaatctgcaggaaacgaagataaagcggccgcataacttcgtatagcatacattatacgaagttatcgcctgttaagatataactgaaaaaagaggggaatttttagatactgaaatgatattttagaataaccagactatatataaggataaattacaaaaaattaactaatagataagatttaaatataaaagatatgcaactagaaaagtcttatcaatctccttatggagtgacgacgttacccaacaatttaccgacttcttcggcgatagccaaagttctctcttcggacaatcttctaccaataacttgaacagcaacaggagcaccgtgataagcctctgggtcgtattcttcttgaaccaaagcatccaattcggaaacagctttaaaagattcgttcttcttatcaatattcttatcagcgaaagtgactgggacgacaacagaggtgaaatccaataagttaataacggaggcgtaaccgtagtatctgaattgatcgtgtctgacagcggcggtaggagtaattggagcgataatagcgtccaattccttaccagctttttcttcagcttcacgccacttttccaagtattccatttgatagttccacttttgtaaatgagtgtcccacaattcgttcatgttaacagccttaatatttgggttcaacaagtccttaatgttagggatggctggctcaccagaggcagaaatgtctctcatgacgtcggcagaaccatcagcagcatagatgtgggaaatcaagtcatgaccgaaatcatgcttgtatggagtccatggagtaacggtgtgaccagccttggccaaagcggcaacggtagtttcgacaccacgtaaaattggtgggtgtggcaagacgttaccgtcgaaattgtaataaccaatgttcaaaccaccattcttaatcttagaggcaatgatgtcagattcagattgtctccatggcattgggatgaccttagagtcgtacttccaaggttcttgacccaagacagatttggtgaacaatctcaagtcttcgacggagtgagtgataggaccaacgacggagtgaacggtttcttgaccttccatagagttagccattttagcatatggcaatctaccgtgagatggtctcaaaccgtataaaaagttgaaagcagctgggactctaatggaaccaccaatgtcagtaccgacaccaataacaccacctctaataccaacaatagcaccttcaccaccagaagaaccaccacaggaccaatttttgtttcttggattgacagttctaccaatgatgttgttgacggtttcacagaccatcaaggtttgtgggacagaggtcttaacgtagaaaacagcaccagcttttctcaacatggtggttaagacggaatcaccttcatcgtatttgtttaaccaggaaatgtaacccatggaggtttcgtaacccttaacacgcaattggtcctttaaagagattggtaaaccgtgtaatggaccaactggtctcttatgcttagcgtagtattcatctaattctctagcttgagctaaagcagcatctgggaagaattcgtgagcacagttggttaattgttgagcaatagcagctctcttacaaaaagccaaagtgacttcaacagaagtcaactcaccagcggccaacttggagaccaaatcagcagcagaggcttcggtaatcttcaattcagcctcagacaaaataccggacttctttgggaaatcaataacggaatcttcggcaggcaaagtttgaaccttccattcgtcaggaatggttttagccaaacgggcacgtttgtcggcggccaattcttcccaggattgtggcattttgtaattaaaacttagattagattgctatgctttctttctaatgagcaagaagtaaaaaaagttgtaatagaacaagaaaaacgaaactgaaacttgagaaattgaagaccatttattaacttaaatatcaatgggaggtcatcgaaagagaaaaaaatcaaaaaaaaaatttttcaagaaaaagaaacgtgataaaaatttttattgcctttttcgacgaagaaaaagaaacgaggcggtctcttttttcttttccaaacctttagtacgggtaattaacgccaccctagaggaagaaagaggggaaatttagtatgctgtgcttgggtgttttgaagtggtacggcgatgcgcggagtccgagaaaatctggaagagtaaaaaaggagtagaaacattttgaagctatggtgtgtgggggatcacttgtgggggattgggtgtgatgtaaggataacttcgtatagcatacattatacgaagttatgcggccgcgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgtactctaatctggtagaaagatgttttacagactgtgtcaatgacttcacaacatcaaagctaaccaataaggaacaaacatgcatcatgaagtgctcagaaaagttcttgaagcatagcgaacgtgtagggcagcgtttccaagag

SEQ ID NO: 3 - DNA, 1132 bp, Artificial Sequence
  Synthetic Polynucleotide
ctcttttttacagatcatcaaggaagtaattatctactttttacaagaattcatgtctaatttacttactgttcaccaaaacttgcctgcattaccagttgacgcaacctccgatgaagtcagaaagaaccttatggatatgtttagagatagacaagctttctccgaacatacttggaaaatgttattatccgtttgtagatcctgggccgcttggtgtaaacttaacaatagaaaatggtttcctgctgaaccagaagacgtcagagattacttactttacttacaagctagaggtttggctgttaaaactatccaacaacacttaggtcaattgaatatgttacacagaagatccggtttaccaagaccatccgattccaacgcagtttcccttgttatgagaagaattagaaaagaaaatgttgacgctggtgaaagagctaaacaagcattagcatttgaaagaaccgatttcgatcaagttagatccttaatggaaaattccgatagatgtcaagatattagaaacttagctttcttaggtattgcttacaacacattattaagaatcgctgaaattgctagaattagagttaaagatatttcaagaaccgatggcggtagaatgttaatccacattggcagaacaaaaaccttagtctccacagcaggcgtcgaaaaagcattatcattaggtgttactaaattagttgaacgttggatttccgtttccggtgttgcagatgacccaaacaactacttattctgtcgtgttagaaaaaatggtgttgccgctccttccgctacctcacaattatccacaagagcattagaaggcatttttgaagctacccacagacttatttatggtgcaaaagacgattccggtcaaagatatttagcttggtctggtcattccgctagagttggtgccgcaagagacatggcaagagctggtgtttctattcctgaaattatgcaagccggtggttggactaatgttaacattgttatgaactatatcagaaacttagattccgaaacaggtgctatggttagattacttgaagacggtgattaagctagctaagatccgctctaaccgaaaaggaaggagttagacaacctg

SEQ ID NO: 4 - DNA, 6376 bp, Artificial Sequence
  Synthetic Polynucleotide
ctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaag

SEQ ID NO: 5 - DNA, 4632 bp, Artificial Sequence
  Synthetic Polynucleotide
cagagcctcttatattcactctgttcctccatcgcctattgagaaacgttggaataaaactctaaaaatatcatctagttggttagtttttattttaccagtacattgtcacttgcggagggaggatgacataaagattgagacgcagtcatttaatgaagtttaaacgcaggtatttgataaagtaatacgatattgaatcatgacgtataaagtgaaatgaacaaatgattacgtaaaaaatgtcgattttctcttgagagactcccatagcctctaagaggccttctactacgttccatatatctaagaatggggccatatccagtggaatcccagcaattatttaaggatcacctatttctcagccgatattttagcaaaatcactaccaatatcagggggcaatagttgatcgcctactttaacaaaaaatgttgctcacgtattaacacaggcaacaaaaaggatattacgcaagaacgtagtatccacatgccatcctccttgttgcatctttttttttccgaaatgattccctttcctgcacaacacgagatctttcacgcatacatcggaaggatcaccccccactcaagtcgttgcattgctaacatgtggcattctgcccatttttttcacgaaaattctctctctataatgaagacccttgtgccctggactctgtaatacttgaaactacttcctcaataatcgcttggagacctacccccacgcttttcaaacaaggcgctagcaaaaagcctgccgatatctccttgccccctccttctgttcgagagaactacgacccgaccaataataatgtcatacaagaaccgccaagaaccaactgctgaaccttagatctccaatacttcagttggagtatgtgaatatataagtacctggtcgactaatcttcttgcatcttttcgtattcttacatcctatgtcgctaatacagttcccgcatagagaagaaagcaaacaaaagtagtcactcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaatctagaatgattagattaaccgtattcctcactgcagtttttgcagcagtcgcttcctgtgttccagttgaattggataagagaaatacaggccatttccaagcatattctggttacaccgtagctagatcaaactttactcaatggattcacgagcaaccagccgtatcatggtactatttgcttcagaatatagactatccagaaggacaattcaagtctgccaagccaggggtcgttgtggcttccccttctacatccgaacctgattacttctaccaatggactagagatactgctatcaccttcttgtcacttatcgcggaagttgaggatcattctttttcaaatactacactagccaaggtggttgaatactacatctctaatacttacacattacaaagagtttccaacccatctggtaacttcgacagtccaaatcacgacggtttgggagaaccaaagtttaatgttgatgatacagcttatactgcatcttggggtagaccacaaaatgatggcccagcgttgagagcatacgcaatttcaagataccttaacgcagtagcaaaacacaacaacggtaagttactgctcgctggacaaaacggtattccttactcttcagcttctgatatctactggaagattatcaagccagatcttcaacatgtgtcaacccattggtctacatctggttttgatttgtgggaagagaatcagggaacacatttctttactgcgttggtccagctaaaagcacttagttacggcattcctttaagtaagacctacaacgatcctggtttcactagttggctagaaaagcaaaaggatgctttaaactcttatatcaacagctctggtttcgtaaactctggcaaaaagcatatagtggagagccctcaactatcttcaagaggagggttggatagcgccacatacattgcagccttaatcacacatgatattggcgacgacgacacttacacacctttcaacgttgacaactcctatgtcttgaactcactgtattaccttctagtcgataacaaaaaccgttacaaaatcaatggtaactacaaggccggtgctgctgttggtagatacccagaggatgtttacaacggtgttgggacatcagaaggcaatccatggcaattagctacagcctacgccggccaaacattttacacactggcttacaactcattgaaaaacaaaaaaaacttagtgattgaaaagttgaactacgacctctacaattctttcatagcagatttatccaagatcgatagttcttacgcatcaaaagactccttgactttgacctacggttctgacaactacaaaaacgtcataaagtcactattacagtttggagattcattcctgaaggtcttgctcgatcacattgatgataatggacaattaacagaagagatcaatagatacacagggttccaggctggtgctgttagtttgacatggtcctctggttcattactttcagcaaaccgtgcgagaaataagttgattgaactattgtagttaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacgc

SEQ ID NO: 6 - DNA, 4363 bp, Artificial Sequence
  Synthetic Polynucleotide
ggccgctccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatctcgagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctgttgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgatcagacttacagttttcctaacagccgttttcgccgccgttgcatcatgtgtcccagtagaattggataagagaaacaccggccatttccaagcatattcaggatacaccgttgcacgttctaatttcacacaatggattcatgagcagcctgctgtgtcctggtactacttattacaaaacattgattatcctgagggacaattcaagtcagcgaaaccaggcgttgtggttgcttctccatccacttcagaaccagactacttctaccagtggacccgtgacacagcaataactttcttatctttgatagcagaagtagaagatcactcattttcaaatacaactctagctaaggttgtcgaatactacatctctaacacatacaccctacaaagagtttctaacccatctggtaatttcgatagcccaaatcacgatggtctgggtgaaccaaagttcaacgttgacgacactgcttacactgcatcatggggcagacctcaaaacgacggtccagccttaagagcttacgcgatctcaagatatttgaacgcagttgccaagcataacaacggtaagctattgctcgcgggtcaaaatggtattccttactcatctgcatcagatatctactggaagattatcaagccagatttacaacatgtaagtactcactggagtacatctggttttgacttatgggaagagaatcaaggtacacatttctttactgcacttgtccagttaaaagctctttcatacggtatacctttgtctaagacatataacgatccaggatttacttcttggttggaaaagcagaaggatgccttgaactcttacatcaattccagcggcttcgtcaactccgggaaaaagcacattgtcgaatctcctcaattatctagtagagggggtcttgatagcgctacttacatcgctgctctaattacacatgatattggtgatgatgatacatacactccttttaacgtagataattcttatgtgctgaactctttatactatctgcttgtagacaacaaaaacagatacaagatcaacgggaactacaaagcaggagctgcagttggtagatacccagaagatgtgtacaatggagtgggaacctcagagggaaacccatggcaattggcgacagcatacgccggccaaaccttttacacactggcttacaattctctcaaaaacaaaaaaaatttggttattgagaagttgaattacgatctatacaactcctttatagctgacttaagtaagattgactcctcttacgcttctaaggattcattgacattgacctacggctcagataactacaaaaatgtcattaagtcacttttacaattcggggattctttcttgaaagtcttgttggaccatattgatgataatggtcagctaacagaggaaatcaacagatatacaggttttcaagctggcgcagtttccctcacttggagtagtggttcactcttatctgcaaacagagccagaaacaagttgatcgaattgctttagttaattaagaagttttgttagaaaataaatcattttttaattgagcattcttattcctattttatttaaatagttttatgtattgttagctacatacaacagtttaaatcaaattttctttttcccaagtccaaaatggaggtttattttgatgacccgcatgcgattatgttttgaaagtataagactacatacatgtacatatatttaaacatgtaaacccgtccattatattgccgggcagacggccggccttatagcctagctttaaggctactttaaaaactttttatttattcatacacatatattatcgaacattcgtataacttaatatcattcaaaaaaaaaaaaaaaaaaaaaagaaaacatatacacatatatatttatgtttatagagagagagagagaaaatttgaatttttgaatcatttgcaaagttatatgttttatacattatttattcattttttttggtgtcgaggacattgtgctgttcagagaaccacttaaaatacgcatcgttctgtaaatatccactttcattaaaaaccttattcacttctaactttgccttcaactccttcttggagttttctcccttttttttctgaacaagctcaaccagatataatggttcgttcttttcgaactttgtctttacatatatttcctcctttgtacctcttctctttcccacataaacagtccccttttcaataaaacgagagaaataccagaaaagtagcgagagaacaaaatatgcgcctaccaaaagcttttgatacgtaacaatctgatctctctcaaattttttatccaagaagaaactcaaaccagctacaacagctatggaataacctatgtacaatttagcatcgagtaaagcgtatgatctctcgtaatttaatctcgcgaaaacagaaggtagggcttcatctaaagcttggttcaactccgggattgaatatacattaataggtttagcagaactcatcttgaacaggcgtctcttttccttacaataacttgtgcttttccttctataattccgtttcaacgtgtacaattgtcattttttgtctggtatgattttgcagaactgaaaaaatctcttaaatgttccgcctcatcaagaaggcatattcctttacaaaagtacattgatcttacaagaagctagctaatggtactatttaaaaaacaactacactccatcaatacataaaattgttatgatagacttgagggacgg

SEQ ID NO: 7 - DNA, 5015 bp, Artificial Sequence
  Synthetic Polynucleotide
cagagcctcttatattcactctgttcctccatcgcctattgagaaacgttggaataaaactctaaaaatatcatctagttggttagtttttattttaccagtacattgtcacttgcggagggaggatgacataaagattgagacgcagtcatttaatgaagtttaaacgcaggtatttgataaagtaatacgatattgaatcatgacgtataaagtgaaatgaacaaatgattacgtaaaaaatgtcgattttctcttgagagactcccatagcctctaagaggccttctactacgttccatatatctaagaatggggccatatccagtggaatcccagcaattatttaaggatcacctatttctcagccgatattttagcaaaatcactaccaatatcagggggcaatagttgatcgcctactttaacaaaaaatgttgctcacgtattaacacaggcaacaaaaaggatattacgcaagaacgtagtatccacatgccatcctccttgttgcatctttttttttccgaaatgattccctttcctgcacaacacgagatctttcacgcatacatcggaaggatcaccccccactcaagtcgttgcattgctaacatgtggcattctgcccatttttttcacgaaaattctctctctataatgaagacccttgtgccctggactctgtaatacttgaaactacttcctcaataatcgcttggagacctacccccacgcttttcaaacaaggcgctagcaaaaagcctgccgatatctccttgccccctccttctgttcgagagaactacgacccgaccaataataatgtcatacaagaaccgccaagaaccaactgctgaaccttagatctccaatacttcagttggagtatgtgaatatataagtacctggtcgactaatcttcttgcatcttttcgtattcttacatcctatgtcgctaatacagttcccgcatagagaagaaagcaaacaaaagtagtcactcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaatctagaatgatcagacttactgttttcctcacagccgtttttgcagcagtagcttcttgtgttccagttgaattggataagagaaatacaggtcatttccaagcttactctggttacactgtggctagatctaacttcacacaatggattcatgaacagcctgccgtgagttggtactatttgctacaaaacattgattaccctgagggtcaattcaaatcagctaagccaggtgttgttgtcgcgagcccatcaacttctgaaccagattacttctaccaatggactagagataccgcaataaccttcttatctctaatcgcagaggtagaagatcactctttttcaaatactaccctggcaaaagtggtcgagtactacatctcaaacacatacaccttgcagagagtctcaaacccatcaggaaacttcgattctcctaatcatgacggcttaggagaaccaaagtttaatgttgacgataccgcttatactgcatcttggggtagaccacagaatgatggccctgccttacgtgcatacgccatttccagatatctcaacgctgtagcgaagcacaacaacggtaagctgcttttagctggtcaaaatgggataccatactcttccgcttcagacatttactggaagattatcaaaccagacttgcagcatgtcagtacacattggtcaacttctggttttgatttgtgggaagagaaccaaggcactcacttctttacagccttggttcaactaaaggcattgtcttacggaatccctttgtccaagacatacaatgatcctggattcactagttggctagaaaagcaaaaggatgcactgaactcatacattaacagttcaggctttgtgaactccggtaaaaagcatattgttgaaagcccacaactatctagcagaggtggtttagattctgcaacctacatagcagccttgatcacacacgacattggggatgacgatacatacacaccattcaacgtcgacaattcatacgttttgaatagcttatactacctactggtagataacaaaaacagatataagatcaatggcaactacaaggccggtgctgccgtaggaagataccctgaagatgtctacaacggagttggtacatcagaaggtaacccatggcaattagcaacagcatatgcgggccagacattttacactttggcttacaattcattgaaaaacaaaaaaaatttagtgatagaaaagcttaactatgacctttacaactctttcattgccgatttatccaagattgattcctcctacgcatcaaaggactccttgacacttacatacggttctgacaactacaaaaatgttatcaagtctctcttgcaatttggtgattctttcttgaaggttttactcgatcatatcgatgataatggtcaactaactgaggaaatcaacagatacactgggttccaagctggagctgtctctttaacatggagttcagggagtttgttatctgctaacagagcgcgtaacaaacttattgagcttctgtagttaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagaccgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtctccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataagagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggc

SEQ ID NO: 8 - DNA, 4771 bp, Artificial Sequence
  Synthetic Polynucleotide
ggccgcgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatcccaatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttcggtcatgacttgatttcccacatctatgctgctgatggttctgccgacgtcatgagagacatttctgcctctggtgagccagccatccctaacattaaggacttgttgaacccaaatattaaggctgttaacatgaacgaattgtgggacactcatttacaaaagtggaactatcaaatggaatacttggaaaagtggcgtgaagctgaagaaaaagctggtaaggaattggacgctattatcgctccaattactcctaccgccgctgtcagacacgatcaattcagatactacggttacgcctccgttattaacttattggatttcacctctgttgtcgtcccagtcactttcgctgataagaatattgataagaagaacgaatcttttaaagctgtttccgaattggatgctttggttcaagaagaatacgacccagaggcttatcacggtgctcctgttgctgttcaagttattggtagaagattgtccgaagagagaactttggctatcgccgaagaagtcggtaaattgttgggtaacgtcgtcactccataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacataacttcgtataatgtatgctatacgaagttatctcgagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctgttgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgattagattaacagtatttcttacagccgttttcgcagccgtcgcatcctgtgttccagtagaattagataagcgtaatacaggacattttcaagcttactctggctatacagttgcgagatctaactttacacaatggattcacgaacagccagcagtttcttggtactatttgctccaaaacatcgactaccctgaaggccaattcaagtctgcaaagccaggagtggtcgtcgcttctcctagtacttcagaaccagattacttctaccagtggacaagagacactgctattaccttcctgagcttaatcgctgaagttgaagatcactctttttctaatacaacactggccaaagtagttgagtactacatctctaacacttacactctacaaagagtgtcaaacccttctgggaacttcgacagcccaaaccatgatggtttgggggagccaaaattcaacgttgatgatacagcctacaccgcatcttggggtagaccacaaaacgacggaccagctttaagagcatacgcaatatctcgttaccttaatgctgttgcaaagcacaataatggaaagttgttgttggctggtcaaaacggtattccttactcttcagcatctgatatctactggaagattatcaagccagatcttcaacacgtatccacacattggtcaacctccggcttcgatttatgggaggaaaatcagggtacacatttcttcaccgctctagtgcaattgaaggctttgagttacggcattccattgtctaagacttacaacgatcctggtttcacctcatggcttgaaaagcagaaggatgccctgaatagctacatcaactcatctggttttgttaactcagggaaaaagcatatagttgaatccccacaactatcatcaagaggaggtttagactccgccacatacattgctgccttgattacacatgatattggggatgatgacacatatactccatttaacgtcgataacagttatgtccttaattccttatactatttgttggtcgataacaaaaatagatacaaaatcaacggcaactacaaggctggcgcagcggtgggtagataccctgaggatgtttacaatggtgtaggtacatctgaaggcaatccatggcaattagcgactgcttacgctggacaaactttctacacacttgcgtacaactcattgaaaaacaaaaaaaacctagtcattgaaaagttgaattacgatctgtacaactctttcatcgcagacctatcaaagattgactcatcttatgcaagtaaagattcactaactttaacctacggtagtgataactacaaaaacgttatcaagtctttactccagtttggtgattcattcttgaaggtgttgttagatcatatagacgacaatggtcaactcacagaggagataaacagatacactggttttcaagcaggagctgtttcacttacttggtcaagtggttctttgctttccgccaacagagccagaaacaagctcatcgaattactatagttaattaagaagttttgttagaaaataaatcattttttaattgagcattcttattcctattttatttaaatagttttatgtattgttagctacatacaacagtttaaatcaaattttctttttcccaagtccaaaatggaggtttattttgatgacccgcatgcgattatgttttgaaagtataagactacatacatgtacatatatttaaacatgtaaacccgtccattatattgccgggcagacggccggccttatagcctagctttaaggctactttaaaaactttttatttattcatacacatatattatcgaacattcgtataacttaatatcattcaaaaaaaaaaaaaaaaaaaaaagaaaacatatacacatatatatttatgtttatagagagagagagagaaaatttgaatttttgaatcatttgcaaagttatatgttttatacattatttattcattttttttggtgtcgaggacattgtgctgttcagagaaccacttaaaatacgcatcgttctgtaaatatccactttcattaaaaaccttattcacttctaactttgccttcaactccttcttggagttttctcccttttttttctgaacaagctcaaccagatataatggttcgttcttttcgaactttgtctttacatatatttcctcctttgtacctcttctctttcccacataaacagtccccttttcaataaaacgagagaaataccagaaaagtagcgagagaacaaaatatgcgcctaccaaaagcttttgatacgtaacaatctgatctctctcaaattttttatccaagaagaaactcaaaccagctacaacagctatggaataacctatgtacaatttagcatcgagtaaagcgtatgatctctcgtaatttaatctcgcgaaaacagaaggtagggcttcatctaaagcttggttcaactccgggattgaatatacattaataggtttagcagaactcatcttgaacaggcgtctcttttccttacaataacttgtgcttttccttctataattccgtttcaacgtgtacaattgtcattttttgtctggtatgattttgcagaactgaaaaaatctcttaaatgttccgcctcatcaagaaggcatattcctttacaaaagtacattgatcttacaagaagctagctaatggtactatttaaaaaacaactacactccatcaatacataaaattgttatgatagacttgagggacgg

SEQ ID NO: 9 - DNA, 8719 bp, Artificial Sequence
  Synthetic Polynucleotide
atcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaatctagaatgagtgaatctccaatgttcgctgccaacggcatgccaaaggtaaatcaaggtgctgaagaagatgtcagaattttaggttacgacccattagcttctccagctctccttcaagtgcaaatcccagccacaccaacttctttggaaactgccaagagaggtagaagagaagctatagatattattaccggtaaagacgacagagttcttgtcattgtcggtccttgttccatccatgatcttgaagccgctcaagaatacgctttgagattaaagaaattgtcagatgaattaaaaggtgatttatccatcattatgagagcatacttggagaagccaagaacaaccgtcggctggaaaggtctaattaatgaccctgatgttaacaacactttcaacatcaacaagggtttgcaatccgctagacaattgtttgtcaacttgacaaatatcggtttgccaattggttctgaaatgcttgataccatttctcctaaatacttggctgatttggtctccttcggtgccattggtgccagaaccaccgaatctcaactgcacagagaattggcctccggtttgtctttcccagttggtttcaagaacggtaccgatggtaccttaaatgttgctgtggatgcttgtcaagccgctgctcattctcaccatttcatgggtgttactaagcatggtgttgctgctatcaccactactaagggtaacgaacactgcttcgttattctaagaggtggtaaaaagggtaccaactacgacgctaagtccgttgcagaagctaaggctcaattgcctgccggttccaacggtctaatgattgactactctcacggtaactccaataaggatttcagaaaccaaccaaaggtcaatgacgttgtttgtgagcaaatcgctaacggtgaaaacgccattaccggtgtcatgattgaatcaaacatcaacgaaggtaaccaaggcatcccagccgaaggtaaagccggcttgaaatatggtgtttccatcactgatgcttgtataggttgggaaactactgaagacgtcttgaggaaattggctgctgctgtcagacaaagaagagaagttaacaagaaatagatgtttttttaatgatatatgtaacgtacattctttcctctaccactgccaattcggtattatttaattgtgtttagcgctatttactaattaactagaaactcaatttttaaaggcaaagctcgctgacctttcactgatttcgtggatgttatactatcagttactcttctgcaaaaaaaaattgagtcatatcgtagctttgggattatttttctctctctccacggctaattaggtgatcatgaaaaaatgaaaaattcatgagaaaagagtcagacatcgaaacatacataagttgatattcctttgatatcgacgactactcaatcaggttttaaaagaaaagaggcagctattgaagtagcagtatccagtttaggttttttaattatttacaagtaaagaaaaagagaatgccggtcgttcacggcggccgcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaactcttttttacagatcatcaaggaagtaattatctactttttacaagaattcatgtctaatttacttactgttcaccaaaacttgcctgcattaccagttgacgcaacctccgatgaagtcagaaagaaccttatggatatgtttagagatagacaagctttctccgaacatacttggaaaatgttattatccgtttgtagatcctgggccgcttggtgtaaacttaacaatagaaaatggtttcctgctgaaccagaagacgtcagagattacttactttacttacaagctagaggtttggctgttaaaactatccaacaacacttaggtcaattgaatatgttacacagaagatccggtttaccaagaccatccgattccaacgcagtttcccttgttatgagaagaattagaaaagaaaatgttgacgctggtgaaagagctaaacaagcattagcatttgaaagaaccgatttcgatcaagttagatccttaatggaaaattccgatagatgtcaagatattagaaacttagctttcttaggtattgcttacaacacattattaagaatcgctgaaattgctagaattagagttaaagatatttcaagaaccgatggcggtagaatgttaatccacattggcagaacaaaaaccttagtctccacagcaggcgtcgaaaaagcattatcattaggtgttactaaattagttgaacgttggatttccgtttccggtgttgcagatgacccaaacaactacttattctgtcgtgttagaaaaaatggtgttgccgctccttccgctacctcacaattatccacaagagcattagaaggcatttttgaagctacccacagacttatttatggtgcaaaagacgattccggtcaaagatatttagcttggtctggtcattccgctagagttggtgccgcaagagacatggcaagagctggtgtttctattcctgaaattatgcaagccggtggttggactaatgttaacattgttatgaactatatcagaaacttagattccgaaacaggtgctatggttagattacttgaagacggtgattaagctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgag

SEQ ID NO: 10 - DNA, 1632 bp, Artificial Sequence
  Synthetic Polynucleotide
ggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttg

SEQ ID NO: 11 - DNA, 4863 bp, Artificial Sequence
  Synthetic Polynucleotide
ctaaattcggccttgctcagagactcctggattttggctaacaacgcagtcccttcgatgcatatagctaggccacaaattatgccaataacggtccatgggttgatgttttcttgaattctttcgtttttcatgctatttgcgtcttcccaagtcccagcgttccagtattcatactgcgcgttagagtggtagccataagagccggcatattggtaattttcagtattaacgttagaacgtggtgaatacgatgtggtccagccttgcctcgttgtgtcatatacgatctttttctttgggtcacaaagaatatcatatgcttgagagatgactttaaatctatgtagtttttcgcttgatgttagcagcagcggtgatttactatcactgttggtaaccttttctgagctaaatatttgaatgttatcggaatggtcagggtggtacaattttacataacgatgatattttttttttaacgacttcttgtccagtttaggatttccagatccggcctttggaatgccaaaaatatcatagggagttggatctgccaactcaggccattgttcatcccttatcgtaagttttctattgccatttttatcgttcgctgtagcatacttagctataaaagtgatttgtgggggacacttttctacacatgataagtgccacttgaataaaaatgggtatacgaacttatggtgtagcataacaaatatattgcaagtagtgacctatggtgtgtagatatacgtacagttagttacgagcctaaagacacaacgtgtttgttaattatactgtcgctgtaatatcttctcttccattatcaccggtcattccttgcaggggcggtagtacccggagaccctgaacttttctttttttttttgcgaaattaaaaagttcattttcaattcgacaatgagatctacaagccattgttttatgttgatgagagccagcttaaagagttctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaatctagaatgaagttcatttccactttcttgaccttcattttggctgctgtctctgtcaccgctgcatctattccatctagtgcatctgtacaattggactcctacaattacgatggttccacattttccggcaagatttatgtcaaaaacatcgcttactctaaaaaggttactgttgtgtacgcagacggttctgacaactggaacaataacggcaacactattgctgcatcattttcaggcccaatctctggatcaaattacgaatactggacattctcagcatcagtgaagggcataaaggagttctacatcaaatacgaagtttcaggtaagacatattacgacaataacaactctgcaaactaccaagtctcaacttctaaacctactacaactactgcagctacaaccacaactacagctccatcaacttctacaacaacccgtccatctagttcagagcctgccaccttccctactggtaattctaccatcagctcttggatcaaaaagcaggaagatatttccagattcgctatgcttagaaacatcaacccacctggttctgccacagggtttatcgccgcatcactctctaccgctggtccagattactactacgcgtggacaagagatgccgctttgacatctaacgttatcgtttacgaatacaacaccacattgtctgggaataagacaattctaaacgtacttaaggattacgtcacattcagtgttaagacacagtctacttcaacagtttgtaattgccttggtgaaccaaagttcaatccagacggcagtggttacacaggtgcttggggtagacctcaaaatgatggtcctgcagaaagagcgactacatttgttctgtttgccgacagctacttgactcaaactaaggatgcctcatacgtcactggtacattaaagccagcaattttcaaagatctcgattacgttgttaacgtctggagtaacggatgtttcgatttatgggaggaggtgaacggagttcatttctacacccttatggttatgagaaaagggctattgttgggggctgatttcgcgaagagaaacggtgactcaactagagcctcaacttactcttctactgcttccacaattgctaacaagatatcaagtttctgggttagctcaaacaactgggtgcaagtatcccaatctgtcacaggaggtgtaagtaaaaaggggttagacgttagcaccctgttagctgcgaatctaggatcagtcgatgatggatttttcactccaggttctgaaaagatattagctacagctgtggcagtcgaagattcctttgccagtctatacccaatcaacaaaaaccttccatcatacttggggaacgctattggaagataccctgaagatacatacaacggtaatggtaactcacaaggcaatccttggtttctggcggttaccggctacgcagagttgtactatagagcaattaaggaatggatttctaatggaggcgttacagtgtcctctatctcattgccatttttcaaaaagttcgatagctctgcaacatccggtaaaaagtacaccgtaggtacttctgacttcaacaatttagcacaaaacattgctcttgctgcagatcgtttcctatctactgtacaactccatgcaccaaacaatggttcattagcagaggaatttgatagaacaacaggtttttctaccggcgctagagatttaacatggtcccacgcctcattgataacagcatcctatgccaaagccggtgctccagctgcataattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacgc

SEQ ID NO: 12 - DNA, 4748 bp, Artificial Sequence
  Synthetic Polynucleotide
ggccgctccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatctcgagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctgttgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgaagtttatctccacgtttttaacctttatcctagcagctgtcagcgtcaccgccgcatcaattccgagttcagcatctgtacaacttgactcttacaattacgatggcagcactttctcagggaaaatttatgtgaaaaacatagcatatagtaagaaggttaccgtggtatatgcagacggttctgataattggaataataatggaaacactattgccgccagtttttccggcccaatttctggttccaattacgagtattggaccttttctgcatcagtaaaaggcatcaaggaattctatattaagtacgaagtttcaggtaagacatattacgataacaataactcagcaaattatcaagtctctacatctaagcccacaacaacaactgctgctaccaccactacaaccgctccttctaccagcaccactaccagaccaagctctagtgaaccggctacctttcctaccggaaacagtaccatctcaagctggatcaaaaagcaagaggacataagtcgttttgctatgttgaggaacattaatcctccaggatccgcgaccggtttcattgcagcatcactaagtactgccgggcctgattattattatgcttggactagagacgctgcattaacatcaaacgtgattgtttatgaatataatacgaccctttccggtaataaaacgatcttgaacgtattaaaagactatgtgacctttagtgtgaagacccaatctacatctacagtgtgtaattgtttgggagaacctaaattcaatccagacggttctgggtacactggtgcctggggtagacctcaaaacgacggtccagcagaaagagcaacaacctttgttctatttgctgactcttatttaacgcaaacaaaggacgcctcatatgttacagggaccctaaaaccagcaattttcaaagacttggattatgttgttaatgtttggagcaacggatgttttgacttgtgggaggaggttaacggtgtacacttttatacattgatggtgatgagaaaagggttgctattgggagcagatttcgctaaaagaaatggtgattctacaagagcgagcacatatagtagcaccgcttcaacaatcgccaataaaatctcatctttctgggtatctagcaacaactgggtacaagtttcccaaagtgttaccggcggtgtgtccaaaaagggtttagacgttagcacacttctagctgctaatttgggtagcgttgatgacgggttttttactccaggtagtgagaagatactggcaaccgcggtggcggttgaagacagctttgcttcattgtatcctataaataaaaatctgccctcttatctgggtaatgcaattggcagatacccagaagatacctacaatggtaatggtaattcccaggggaacccatggtttttggctgttacaggctacgcagaactttattaccgtgcaatcaaggaatggatttcaaatggcggcgtcactgtcagtagtataagtttgcccttttttaagaaatttgattcctcagcaacgtctggtaaaaaatacaccgtaggtactagtgatttcaataatttggcccaaaatattgcgcttgctgctgacaggtttcttagtaccgttcagttgcacgctccaaataatggctcattggctgaagaatttgatcgtacgacaggtttctccactggtgctagggatttgacttggagtcatgcctccttaatcacagcaagctatgctaaagctggtgcacctgctgcttagttaattaatttaccagcttactatccttcttgaaaatatgcactctatatcttttagttcttaattgcaacacatagatttgctgtataacgaattttatgctatttttttaatttggagttcggtgatgaaagtgtcacagcgaatttcctcacatgtagggaccgaattgtttacaagttctctgtaccaccatggagacatcaaagattgaaaatctatggaaagatatggacggtagcaacaagaatatagcacgagccgcggagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctcaggccggcagcacgcagcacgctgtatttacgtatttaattttatatatttgtgcatacactactagggaagacttgaaaaaaacctaggaaatgaaaaaacgacacaggaagtcccgtatttactattttttccttccttttgatggggcagggcggaaatagaggataggataagcctactgcttagctgtttccgtctctacttcggtagttgtctcaattgtcgtttcagtattacctttagagccgctagacgatggttgagctatttgttgagggaaaactaagttcatgtaacacacgcataacccgattaaactcatgaatagcttgattgcaggaggctggtccattggagatggtgccttattttccttataggcaacgatgatgtcttcgtcggtgttcaggtagtagtgtacactctgaatcagggagaaccaggcaatgaacttgttcctcaagaaaatagcggccataggcatggattggttaaccacaccagatatgcttggtgtggcagaatatagtccttttggtggcgcaattttcttgtacctgtggtagaaagggagcggttgaactgttagtatatattggcaatatcagcaaatttgaaagaaaattgtcggtgaaaaacatacgaaacacaaaggtcgggccttgcaacgttattcaaagtcattgtttagttgaggaggtagcagcggagtatatgtattccttttttttgcctatggatgttgtaccatgcccattctgctcaagcttttgttaaaattatttttcagtattttttcttccatgttgcgcgttacgagaacagaagcgacagataaccgcaatcatacaactagcgctactgcggggtgtaaaaagcacaagaactaagccaagatcacaacagttat

SEQ ID NO: 13 - DNA, 4260 bp, Artificial Sequence
  Synthetic Polynucleotide
tcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaatctagaatgaagttcatttccactttcttgaccttcattttggctgctgtctctgtcaccgctgcatctattccatctagtgcatctgtacaattggactcctacaattacgatggttccacattttccggcaagatttatgtcaaaaacatcgcttactctaaaaaggttactgttgtgtacgcagacggttctgacaactggaacaataacggcaacactattgctgcatcattttcaggcccaatctctggatcaaattacgaatactggacattctcagcatcagtgaagggcataaaggagttctacatcaaatacgaagtttcaggtaagacatattacgacaataacaactctgcaaactaccaagtctcaacttctaaacctactacaactactgcagctacaaccacaactacagctccatcaacttctacaacaacccgtccatctagttcagagcctgccaccttccctactggtaattctaccatcagctcttggatcaaaaagcaggaagatatttccagattcgctatgcttagaaacatcaacccacctggttctgccacagggtttatcgccgcatcactctctaccgctggtccagattactactacgcgtggacaagagatgccgctttgacatctaacgttatcgtttacgaatacaacaccacattgtctgggaataagacaattctaaacgtacttaaggattacgtcacattcagtgttaagacacagtctacttcaacagtttgtaattgccttggtgaaccaaagttcaatccagacggcagtggttacacaggtgcttggggtagacctcaaaatgatggtcctgcagaaagagcgactacatttgttctgtttgccgacagctacttgactcaaactaaggatgcctcatacgtcactggtacattaaagccagcaattttcaaagatctcgattacgttgttaacgtctggagtaacggatgtttcgatttatgggaggaggtgaacggagttcatttctacacccttatggttatgagaaaagggctattgttgggggctgatttcgcgaagagaaacggtgactcaactagagcctcaacttactcttctactgcttccacaattgctaacaagatatcaagtttctgggttagctcaaacaactgggtgcaagtatcccaatctgtcacaggaggtgtaagtaaaaaggggttagacgttagcaccctgttagctgcgaatctaggatcagtcgatgatggatttttcactccaggttctgaaaagatattagctacagctgtggcagtcgaagattcctttgccagtctatacccaatcaacaaaaaccttccatcatacttggggaacgctattggaagataccctgaagatacatacaacggtaatggtaactcacaaggcaatccttggtttctggcggttaccggctacgcagagttgtactatagagcaattaaggaatggatttctaatggaggcgttacagtgtcctctatctcattgccatttttcaaaaagttcgatagctctgcaacatccggtaaaaagtacaccgtaggtacttctgacttcaacaatttagcacaaaacattgctcttgctgcagatcgtttcctatctactgtacaactccatgcaccaaacaatggttcattagcagaggaatttgatagaacaacaggtttttctaccggcgctagagatttaacatggtcccacgcctcattgataacagcatcctatgccaaagccggtgctccagctgcataattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagaccgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtctccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataagagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggc

SEQ ID NO: 14 - DNA, 5008 bp, Artificial Sequence
  Synthetic Polynucleotide
ggccgcgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatcccaatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttcggtcatgacttgatttcccacatctatgctgctgatggttctgccgacgtcatgagagacatttctgcctctggtgagccagccatccctaacattaaggacttgttgaacccaaatattaaggctgttaacatgaacgaattgtgggacactcatttacaaaagtggaactatcaaatggaatacttggaaaagtggcgtgaagctgaagaaaaagctggtaaggaattggacgctattatcgctccaattactcctaccgccgctgtcagacacgatcaattcagatactacggttacgcctccgttattaacttattggatttcacctctgttgtcgtcccagtcactttcgctgataagaatattgataagaagaacgaatcttttaaagctgtttccgaattggatgctttggttcaagaagaatacgacccagaggcttatcacggtgctcctgttgctgttcaagttattggtagaagattgtccgaagagagaactttggctatcgccgaagaagtcggtaaattgttgggtaacgtcgtcactccataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacataacttcgtataatgtatgctatacgaagttatctcgagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctgttgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgaagtttatctccacgtttttaacctttatcctagcagctgtcagcgtcaccgccgcatcaattccgagttcagcatctgtacaacttgactcttacaattacgatggcagcactttctcagggaaaatttatgtgaaaaacatagcatatagtaagaaggttaccgtggtatatgcagacggttctgataattggaataataatggaaacactattgccgccagtttttccggcccaatttctggttccaattacgagtattggaccttttctgcatcagtaaaaggcatcaaggaattctatattaagtacgaagtttcaggtaagacatattacgataacaataactcagcaaattatcaagtctctacatctaagcccacaacaacaactgctgctaccaccactacaaccgctccttctaccagcaccactaccagaccaagctctagtgaaccggctacctttcctaccggaaacagtaccatctcaagctggatcaaaaagcaagaggacataagtcgttttgctatgttgaggaacattaatcctccaggatccgcgaccggtttcattgcagcatcactaagtactgccgggcctgattattattatgcttggactagagacgctgcattaacatcaaacgtgattgtttatgaatataatacgaccctttccggtaataaaacgatcttgaacgtattaaaagactatgtgacctttagtgtgaagacccaatctacatctacagtgtgtaattgtttgggagaacctaaattcaatccagacggttctgggtacactggtgcctggggtagacctcaaaacgacggtccagcagaaagagcaacaacctttgttctatttgctgactcttatttaacgcaaacaaaggacgcctcatatgttacagggaccctaaaaccagcaattttcaaagacttggattatgttgttaatgtttggagcaacggatgttttgacttgtgggaggaggttaacggtgtacacttttatacattgatggtgatgagaaaagggttgctattgggagcagatttcgctaaaagaaatggtgattctacaagagcgagcacatatagtagcaccgcttcaacaatcgccaataaaatctcatctttctgggtatctagcaacaactgggtacaagtttcccaaagtgttaccggcggtgtgtccaaaaagggtttagacgttagcacacttctagctgctaatttgggtagcgttgatgacgggttttttactccaggtagtgagaagatactggcaaccgcggtggcggttgaagacagctttgcttcattgtatcctataaataaaaatctgccctcttatctgggtaatgcaattggcagatacccagaagatacctacaatggtaatggtaattcccaggggaacccatggtttttggctgttacaggctacgcagaactttattaccgtgcaatcaaggaatggatttcaaatggcggcgtcactgtcagtagtataagtttgcccttttttaagaaatttgattcctcagcaacgtctggtaaaaaatacaccgtaggtactagtgatttcaataatttggcccaaaatattgcgcttgctgctgacaggtttcttagtaccgttcagttgcacgctccaaataatggctcattggctgaagaatttgatcgtacgacaggtttctccactggtgctagggatttgacttggagtcatgcctccttaatcacagcaagctatgctaaagctggtgcacctgctgcttagttaattaatttaccagcttactatccttcttgaaaatatgcactctatatcttttagttcttaattgcaacacatagatttgctgtataacgaattttatgctatttttttaatttggagttcggtgatgaaagtgtcacagcgaatttcctcacatgtagggaccgaattgtttacaagttctctgtaccaccatggagacatcaaagattgaaaatctatggaaagatatggacggtagcaacaagaatatagcacgagccgcggagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctcaggccggcagcacgcagcacgctgtatttacgtatttaattttatatatttgtgcatacactactagggaagacttgaaaaaaacctaggaaatgaaaaaacgacacaggaagtcccgtatttactattttttccttccttttgatggggcagggcggaaatagaggataggataagcctactgcttagctgtttccgtctctacttcggtagttgtctcaattgtcgtttcagtattacctttagagccgctagacgatggttgagctatttgttgagggaaaactaagttcatgtaacacacgcataacccgattaaactcatgaatagcttgattgcaggaggctggtccattggagatggtgccttattttccttataggcaacgatgatgtcttcgtcggtgttcaggtagtagtgtacactctgaatcagggagaaccaggcaatgaacttgttcctcaagaaaatagcggccataggcatggattggttaaccacaccagatatgcttggtgtggcagaatatagtccttttggtggcgcaattttcttgtacctgtggtagaaagggagcggttgaactgttagtatatattggcaatatcagcaaatttgaaagaaaattgtcggtgaaaaacatacgaaacacaaaggtcgggccttgcaacgttattcaaagtcattgtttagttgaggaggtagcagcggagtatatgtattccttttttttgcctatggatgttgtaccatgcccattctga

SEQ ID NO: 15 - DNA, 4881 bp, Artificial Sequence
  Synthetic Polynucleotide
ctaaattcggccttgctcagagactcctggattttggctaacaacgcagtcccttcgatgcatatagctaggccacaaattatgccaataacggtccatgggttgatgttttcttgaattctttcgtttttcatgctatttgcgtcttcccaagtcccagcgttccagtattcatactgcgcgttagagtggtagccataagagccggcatattggtaattttcagtattaacgttagaacgtggtgaatacgatgtggtccagccttgcctcgttgtgtcatatacgatctttttctttgggtcacaaagaatatcatatgcttgagagatgactttaaatctatgtagtttttcgcttgatgttagcagcagcggtgatttactatcactgttggtaaccttttctgagctaaatatttgaatgttatcggaatggtcagggtggtacaattttacataacgatgatattttttttttaacgacttcttgtccagtttaggatttccagatccggcctttggaatgccaaaaatatcatagggagttggatctgccaactcaggccattgttcatcccttatcgtaagttttctattgccatttttatcgttcgctgtagcatacttagctataaaagtgatttgtgggggacacttttctacacatgataagtgccacttgaataaaaatgggtatacgaacttatggtgtagcataacaaatatattgcaagtagtgacctatggtgtgtagatatacgtacagttagttacgagcctaaagacacaacgtgtttgttaattatactgtcgctgtaatatcttctcttccattatcaccggtcattccttgcaggggcggtagtacccggagaccctgaacttttctttttttttttgcgaaattaaaaagttcattttcaattcgacaatgagatctacaagccattgttttatgttgatgagagccagcttaaagagttctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaatctagaatgcagttattcaacttaccacttaaggtatctttctttctagtcttatcttacttttcattgttagtatcagctgcctctataccaagttcagcatccgtacaactagattcatacaattacgacggttcaacattctcaggaaagatatacgtgaaaaatattgcttacagcaaaaaggttactgtgatttacgcagatgggtcagacaactggaataacaatggaaacacaattgctgcttcctattctgcccctatttctggatctaactacgaatactggactttttcagcgagtataaacggaattaaggaattctatatcaaatatgaagtctctggtaagacctactacgataacaacaactccgcaaactaccaagttagcacatcaaagccaaccacaacaactgctactgcgacaactacaaccgcaccaagcacttctactacaacacctcctagttcatctgagccagcaactttcccaactggtaattccactatttcttcttggatcaaaaaacaagagggtatctcaagattcgccatgcttagaaatatcaatcctccaggctctgcaacaggattcattgcagcatctttatcaactgcggggccagactactactacgcctggactagagatgcagctttgacatcaaatgtgattgtttatgaatacaacacaactttgtccggtaacaagacaatcttgaacgtcttgaaggattatgtgacattctctgtcaagactcaatctacatcaacagtttgtaactgtctcggcgaaccaaagttcaaccctgatggtagtggttacactggtgcttggggtagaccacaaaacgatggtccagcagagagagctacaactttcatcttgtttgctgactcttacctaacacaaaccaaggatgcaagctacgttactggaacactaaagcctgcaatctttaaagacctggactatgttgtaaacgtttggtcaaatggctgcttcgatctatgggaggaagtgaacggtgttcacttctacacattaatggtcatgagaaagggactcttgcttggtgcagactttgctaagagaaacggtgattctacacgtgcctccacttactcctccacagcttcaaccattgccaacaaaatctcttctttctgggtcagctcaaataactggattcaagtttctcaatcagttactggtggtgtttctaaaaagggcctggatgtgtcaaccttgcttgctgccaatttgggcagtgttgatgacgggttcttcaccccaggttctgaaaagatcctcgccaccgcagttgccgttgaagattcatttgctagtttatacccaatcaacaaaaatctaccatcataccttggaaattcaatcggtagatatccagaggatacatacaacggtaatggaaactctcagggtaacccttggtttcttgcagttacagggtacgctgaactgtactacagagcgattaaggaatggattggtaatggcggcgtaactgttagttctatttctctacctttcttcaaaaagttcgatagttctgcaacatctggtaaaaagtacacagtcggcacttccgattttaacaatttagctcagaacatagcactggcagctgatcgtttcttgagtacagtccaattgcatgcccataacaacggtagtttggctgaagagtttgatagaaccaccggtttatcaaccggcgccagagatttaacatggtcccatgcgtctttgataactgcttcttacgccaaggctggggcaccagctgcctgattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacgc

SEQ ID NO: 16 - DNA, 4824 bp, Artificial Sequence
  Synthetic Polynucleotide
ggccgctccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatctcgagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctgttgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgcagctgttcaacttgccattaaaggtttcattctttttggtcctatcatactttagtttgttggtgtcagccgcatctattccatcttcagcatctgtacaattagactcctacaattacgacggctctacattcagcggaaagatttacgtgaaaaatattgcgtacagcaaaaaagtaactgttatctatgccgacggatcagataactggaacaacaatggaaacactatcgctgccagttactctgcaccaatttcaggttctaactacgaatattggacattctcagcctccatcaatggcattaaggaattctacataaagtacgaagtttccggtaagacttactacgataacaacaattctgcaaactatcaagtatcaacatcaaaacctactaccaccaccgccacagctacaactacaactgcaccttcaacatctaccacaaccccaccatcttctagcgaaccagctacattcccaactggcaattctactatttctagttggatcaaaaaacaagagggtatttccagattcgcaatgttgagaaacataaatccaccaggatcagcaactggattcatcgcagcttctttgtccacagcggggccagattactactacgcatggaccagagatgctgctttgacaagtaacgttattgtttacgaatacaataccactttgtccggtaacaagactattcttaacgtcctaaaggattacgttacattctctgttaagactcagtctacatccacagtctgcaattgtttgggtgaaccaaagttcaacccagatggctctggatacacaggtgcctggggtcgtccacaaaacgatgggcctgccgagagagccactacatttatcctatttgctgactcataccttacacaaacaaaagatgcatcctacgtgactggaacattaaagcctgcaatcttcaaagacctggattacgttgtcaacgtgtggtctaacggctgtttcgatctatgggaagaggttaacggcgtgcacttctacactctaatggtcatgagaaagggtctgttgttaggtgcagattttgctaagagaaacggtgattctacacgtgcttctacctactcctcaacagcatcaactattgcgaacaagatttcttcattttgggtttcaagtaataactggatacaagtatctcaaagcgttacagggggtgtctcaaaaaagggtcttgatgtttctacattactggctgctaatcttgggtctgttgatgacggtttcttcacccctggttctgaaaagatcctcgctaccgccgtcgcggttgaggatagttttgcttcactctatcctataaacaaaaaccttccttcatacttaggaaacagtatcggtagatacccagaggatacatacaatggtaatggcaattcacagggaaatccatggttccttgctgttacagggtacgcagaactttactatagagctattaaggaatggatcggcaacggcggtgtgacagtttcctcaatctcattgccatttttcaaaaagtttgactccagcgcgacatctggtaaaaagtatactgtggggacttctgatttcaacaatttggctcaaaacattgccttagctgccgacagattcttatctaccgtacaactccatgcacataacaatggtagtttggcagaggaatttgatagaactacaggactctctacaggtgcgagagatttaacttggtcacatgcaagtttaattacagcctcttacgcaaaggctggtgctcctgctgcataattaattaatttaccagcttactatccttcttgaaaatatgcactctatatcttttagttcttaattgcaacacatagatttgctgtataacgaattttatgctatttttttaatttggagttcggtgatgaaagtgtcacagcgaatttcctcacatgtagggaccgaattgtttacaagttctctgtaccaccatggagacatcaaagattgaaaatctatggaaagatatggacggtagcaacaagaatatagcacgagccgcggagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctcaggccggcagcacgcagcacgctgtatttacgtatttaattttatatatttgtgcatacactactagggaagacttgaaaaaaacctaggaaatgaaaaaacgacacaggaagtcccgtatttactattttttccttccttttgatggggcagggcggaaatagaggataggataagcctactgcttagctgtttccgtctctacttcggtagttgtctcaattgtcgtttcagtattacctttagagccgctagacgatggttgagctatttgttgagggaaaactaagttcatgtaacacacgcataacccgattaaactcatgaatagcttgattgcaggaggctggtccattggagatggtgccttattttccttataggcaacgatgatgtcttcgtcggtgttcaggtagtagtgtacactctgaatcagggagaaccaggcaatgaacttgttcctcaagaaaatagcggccataggcatggattggttaaccacaccagatatgcttggtgtggcagaatatagtccttttggtggcgcaattttcttgtacctgtggtagaaagggagcggttgaactgttagtatatattggcaatatcagcaaatttgaaagaaaattgtcggtgaaaaacatacgaaacacaaaggtcgggccttgcaacgttattcaaagtcattgtttagttgaggaggtagcagcggagtatatgtattccttttttttgcctatggatgttgtaccatgcccattctgctcaagcttttgttaaaattatttttcagtattttttcttccatgttgcgcgttacgagaacagaagcgacagataaccgcaatcatacaactagcgctactgcggggtgtaaaaagcacaagaactaagccaagatcacaacagttatcgataaaatagcagtgtttgcatggccattgagaaggacaacattggcgtgcggcatg

SEQ ID NO: 17 - DNA, 5264 bp, Artificial Sequence
  Synthetic Polynucleotide
ctaaattcggccttgctcagagactcctggattttggctaacaacgcagtcccttcgatgcatatagctaggccacaaattatgccaataacggtccatgggttgatgttttcttgaattctttcgtttttcatgctatttgcgtcttcccaagtcccagcgttccagtattcatactgcgcgttagagtggtagccataagagccggcatattggtaattttcagtattaacgttagaacgtggtgaatacgatgtggtccagccttgcctcgttgtgtcatatacgatctttttctttgggtcacaaagaatatcatatgcttgagagatgactttaaatctatgtagtttttcgcttgatgttagcagcagcggtgatttactatcactgttggtaaccttttctgagctaaatatttgaatgttatcggaatggtcagggtggtacaattttacataacgatgatattttttttttaacgacttcttgtccagtttaggatttccagatccggcctttggaatgccaaaaatatcatagggagttggatctgccaactcaggccattgttcatcccttatcgtaagttttctattgccatttttatcgttcgctgtagcatacttagctataaaagtgatttgtgggggacacttttctacacatgataagtgccacttgaataaaaatgggtatacgaacttatggtgtagcataacaaatatattgcaagtagtgacctatggtgtgtagatatacgtacagttagttacgagcctaaagacacaacgtgtttgttaattatactgtcgctgtaatatcttctcttccattatcaccggtcattccttgcaggggcggtagtacccggagaccctgaacttttctttttttttttgcgaaattaaaaagttcattttcaattcgacaatgagatctacaagccattgttttatgttgatgagagccagcttaaagagttctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaatctagaatgcagttattcaacttaccacttaaggtatctttctttctagtcttatcttacttttcattgttagtatcagctgcctctataccaagttcagcatccgtacaactagattcatacaattacgacggttcaacattctcaggaaagatatacgtgaaaaatattgcttacagcaaaaaggttactgtgatttacgcagatgggtcagacaactggaataacaatggaaacacaattgctgcttcctattctgcccctatttctggatctaactacgaatactggactttttcagcgagtataaacggaattaaggaattctatatcaaatatgaagtctctggtaagacctactacgataacaacaactccgcaaactaccaagttagcacatcaaagccaaccacaacaactgctactgcgacaactacaaccgcaccaagcacttctactacaacacctcctagttcatctgagccagcaactttcccaactggtaattccactatttcttcttggatcaaaaaacaagagggtatctcaagattcgccatgcttagaaatatcaatcctccaggctctgcaacaggattcattgcagcatctttatcaactgcggggccagactactactacgcctggactagagatgcagctttgacatcaaatgtgattgtttatgaatacaacacaactttgtccggtaacaagacaatcttgaacgtcttgaaggattatgtgacattctctgtcaagactcaatctacatcaacagtttgtaactgtctcggcgaaccaaagttcaaccctgatggtagtggttacactggtgcttggggtagaccacaaaacgatggtccagcagagagagctacaactttcatcttgtttgctgactcttacctaacacaaaccaaggatgcaagctacgttactggaacactaaagcctgcaatctttaaagacctggactatgttgtaaacgtttggtcaaatggctgcttcgatctatgggaggaagtgaacggtgttcacttctacacattaatggtcatgagaaagggactcttgcttggtgcagactttgctaagagaaacggtgattctacacgtgcctccacttactcctccacagcttcaaccattgccaacaaaatctcttctttctgggtcagctcaaataactggattcaagtttctcaatcagttactggtggtgtttctaaaaagggcctggatgtgtcaaccttgcttgctgccaatttgggcagtgttgatgacgggttcttcaccccaggttctgaaaagatcctcgccaccgcagttgccgttgaagattcatttgctagtttatacccaatcaacaaaaatctaccatcataccttggaaattcaatcggtagatatccagaggatacatacaacggtaatggaaactctcagggtaacccttggtttcttgcagttacagggtacgctgaactgtactacagagcgattaaggaatggattggtaatggcggcgtaactgttagttctatttctctacctttcttcaaaaagttcgatagttctgcaacatctggtaaaaagtacacagtcggcacttccgattttaacaatttagctcagaacatagcactggcagctgatcgtttcttgagtacagtccaattgcatgcccataacaacggtagtttggctgaagagtttgatagaaccaccggtttatcaaccggcgccagagatttaacatggtcccatgcgtctttgataactgcttcttacgccaaggctggggcaccagctgcctgattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagaccgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtctccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataagagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggc

SEQ ID NO: 18 - DNA, 5026 bp, Artificial Sequence
  Synthetic Polynucleotide
ggccgcgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatcccaatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttcggtcatgacttgatttcccacatctatgctgctgatggttctgccgacgtcatgagagacatttctgcctctggtgagccagccatccctaacattaaggacttgttgaacccaaatattaaggctgttaacatgaacgaattgtgggacactcatttacaaaagtggaactatcaaatggaatacttggaaaagtggcgtgaagctgaagaaaaagctggtaaggaattggacgctattatcgctccaattactcctaccgccgctgtcagacacgatcaattcagatactacggttacgcctccgttattaacttattggatttcacctctgttgtcgtcccagtcactttcgctgataagaatattgataagaagaacgaatcttttaaagctgtttccgaattggatgctttggttcaagaagaatacgacccagaggcttatcacggtgctcctgttgctgttcaagttattggtagaagattgtccgaagagagaactttggctatcgccgaagaagtcggtaaattgttgggtaacgtcgtcactccataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacataacttcgtataatgtatgctatacgaagttatctcgagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctgttgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgcagctgttcaacttgccattaaaggtttcattctttttggtcctatcatactttagtttgttggtgtcagccgcatctattccatcttcagcatctgtacaattagactcctacaattacgacggctctacattcagcggaaagatttacgtgaaaaatattgcgtacagcaaaaaagtaactgttatctatgccgacggatcagataactggaacaacaatggaaacactatcgctgccagttactctgcaccaatttcaggttctaactacgaatattggacattctcagcctccatcaatggcattaaggaattctacataaagtacgaagtttccggtaagacttactacgataacaacaattctgcaaactatcaagtatcaacatcaaaacctactaccaccaccgccacagctacaactacaactgcaccttcaacatctaccacaaccccaccatcttctagcgaaccagctacattcccaactggcaattctactatttctagttggatcaaaaaacaagagggtatttccagattcgcaatgttgagaaacataaatccaccaggatcagcaactggattcatcgcagcttctttgtccacagcggggccagattactactacgcatggaccagagatgctgctttgacaagtaacgttattgtttacgaatacaataccactttgtccggtaacaagactattcttaacgtcctaaaggattacgttacattctctgttaagactcagtctacatccacagtctgcaattgtttgggtgaaccaaagttcaacccagatggctctggatacacaggtgcctggggtcgtccacaaaacgatgggcctgccgagagagccactacatttatcctatttgctgactcataccttacacaaacaaaagatgcatcctacgtgactggaacattaaagcctgcaatcttcaaagacctggattacgttgtcaacgtgtggtctaacggctgtttcgatctatgggaagaggttaacggcgtgcacttctacactctaatggtcatgagaaagggtctgttgttaggtgcagattttgctaagagaaacggtgattctacacgtgcttctacctactcctcaacagcatcaactattgcgaacaagatttcttcattttgggtttcaagtaataactggatacaagtatctcaaagcgttacagggggtgtctcaaaaaagggtcttgatgtttctacattactggctgctaatcttgggtctgttgatgacggtttcttcacccctggttctgaaaagatcctcgctaccgccgtcgcggttgaggatagttttgcttcactctatcctataaacaaaaaccttccttcatacttaggaaacagtatcggtagatacccagaggatacatacaatggtaatggcaattcacagggaaatccatggttccttgctgttacagggtacgcagaactttactatagagctattaaggaatggatcggcaacggcggtgtgacagtttcctcaatctcattgccatttttcaaaaagtttgactccagcgcgacatctggtaaaaagtatactgtggggacttctgatttcaacaatttggctcaaaacattgccttagctgccgacagattcttatctaccgtacaactccatgcacataacaatggtagtttggcagaggaatttgatagaactacaggactctctacaggtgcgagagatttaacttggtcacatgcaagtttaattacagcctcttacgcaaaggctggtgctcctgctgcataattaattaatttaccagcttactatccttcttgaaaatatgcactctatatcttttagttcttaattgcaacacatagatttgctgtataacgaattttatgctatttttttaatttggagttcggtgatgaaagtgtcacagcgaatttcctcacatgtagggaccgaattgtttacaagttctctgtaccaccatggagacatcaaagattgaaaatctatggaaagatatggacggtagcaacaagaatatagcacgagccgcggagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctcaggccggcagcacgcagcacgctgtatttacgtatttaattttatatatttgtgcatacactactagggaagacttgaaaaaaacctaggaaatgaaaaaacgacacaggaagtcccgtatttactattttttccttccttttgatggggcagggcggaaatagaggataggataagcctactgcttagctgtttccgtctctacttcggtagttgtctcaattgtcgtttcagtattacctttagagccgctagacgatggttgagctatttgttgagggaaaactaagttcatgtaacacacgcataacccgattaaactcatgaatagcttgattgcaggaggctggtccattggagatggtgccttattttccttataggcaacgatgatgtcttcgtcggtgttcaggtagtagtgtacactctgaatcagggagaaccaggcaatgaacttgttcctcaagaaaatagcggccataggcatggattggttaaccacaccagatatgcttggtgtggcagaatatagtccttttggtggcgcaattttcttgtacctgtggtagaaagggagcggttgaactgttagtatatattggcaatatcagcaaatttgaaagaaaattgtcggtgaaaaacatacgaaacacaaaggtcgggccttgcaacgttattcaaagtcattgtttagttgaggaggtagcagcggagtatatgtattccttttttttgcctatggatgttgtaccatgcccattctga

SEQ ID NO: 19 - DNA, 4884 bp, Artificial Sequence
  Synthetic Polynucleotide
ctaaattcggccttgctcagagactcctggattttggctaacaacgcagtcccttcgatgcatatagctaggccacaaattatgccaataacggtccatgggttgatgttttcttgaattctttcgtttttcatgctatttgcgtcttcccaagtcccagcgttccagtattcatactgcgcgttagagtggtagccataagagccggcatattggtaattttcagtattaacgttagaacgtggtgaatacgatgtggtccagccttgcctcgttgtgtcatatacgatctttttctttgggtcacaaagaatatcatatgcttgagagatgactttaaatctatgtagtttttcgcttgatgttagcagcagcggtgatttactatcactgttggtaaccttttctgagctaaatatttgaatgttatcggaatggtcagggtggtacaattttacataacgatgatattttttttttaacgacttcttgtccagtttaggatttccagatccggcctttggaatgccaaaaatatcatagggagttggatctgccaactcaggccattgttcatcccttatcgtaagttttctattgccatttttatcgttcgctgtagcatacttagctataaaagtgatttgtgggggacacttttctacacatgataagtgccacttgaataaaaatgggtatacgaacttatggtgtagcataacaaatatattgcaagtagtgacctatggtgtgtagatatacgtacagttagttacgagcctaaagacacaacgtgtttgttaattatactgtcgctgtaatatcttctcttccattatcaccggtcattccttgcaggggcggtagtacccggagaccctgaacttttctttttttttttgcgaaattaaaaagttcattttcaattcgacaatgagatctacaagccattgttttatgttgatgagagccagcttaaagagttctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaatctagaatgaaacttatgaatccatctatgaaggcatacgttttctttatcttaagctacttctctttactcgttagctcagctgcggtgccaacctctgccgccgtacaagttgagtcatacaattatgacggtaccactttttcaggtagaatattcgtcaaaaacattgcctactcaaaggtcgtaacagttatctactccgatggatcagataactggaacaataacaacaacaaagtttctgcagcttactcagaagcaatttctgggtctaactacgaatactggacattctccgcaaagttatccggaattaaacagttttatgtcaaatacgaagtttctggttcaacatattacgacaacaacggtaccaaaaactaccaagtccaagcaacctcagcgacatctacaacagctactgcaaccacaactacagctactggcacaacaactacttctacaggtccaactagtactgcatccgtatcattccctaccggtaactcaacaatttcttcctggataaaaaatcaagaggaaatcagccgttttgctatgttgagaaatatcaatccacctgggtctgccacagggttcatagccgcatctctgtccacagccggcccagattactattactcttggactagagattcagcactaacagctaatgtgatcgcttacgaatacaacacaacattcactggaaacaccacccttcttaagtacttgaaagattacgttacattttctgtcaaaagccaatctgtatctaccgtttgtaactgtctgggagaaccaaagttcaacgctgatggtagttcttttacaggtccatggggcagaccacaaaacgacggaccagcagagagagctgttacttttatgttgattgctgacagctacttgactcaaactaaggacgcatcctacgttaccggtacattaaagccagcaatcttcaaagatcttgattacgtagtttctgtttggtctaacggttgctacgatttatgggaagaggttaatggtgttcatttctatactctcatggtcatgagaaagggtttgatcttaggtgccgacttcgctgctagaaatggtgactctagtagagcttcaacctacaagcaaactgcatcaacaatggaatcaaagatcagttctttttggtcagattctaacaactacgtccaagtttctcaatcagttaccgccggagtgtcaaaaaagggactagatgttagtacactattggcggccaacattggtagtctgcctgatggctttttcactccaggctccgaaaagatattggctacagcagtggcgttagaaaatgcattcgcatccttgtacccaattaactctaacctaccttcttacttgggtaactcaattggaagatatcctgaggatacatacaacggtaatggcaactctcaggggaatccatggttccttgccgtcaacgcatacgcagaactttactacagagctattaaggaatggattagtaatggcaaggtgacagtatccaatatctcactacctttcttcaaaaagtttgattcttccgccacttctggaaagacatacactgctggtacatcagatttcaataacttggctcagaacattgctttaggcgccgatagattcctgtctactgttaagttccacgcatacactaacgggagtctatcagaagagtacgatagatctaccggtatgagtactggggctcgtgatttaacatggtcccatgcttcattgatcacagtggcgtacgcaaaggccggtagtcctgcagcttagttaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacgc

SEQ ID NO: 20 - DNA, 1790 bp, Saccharomyces cerevisiae
ggaagagctcctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatctttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcgcacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagagccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgcggccgccagcacgcagcacgctgtatttacgtatttaattttatatatttgtgcatacactactagggaagacttgaaaaaaacctaggaaatgaaaaaacgacacaggaagtcccgtatttactattttttccttccttttgatggggcagggcggaaatagaggataggataagcctactgc

SEQ ID NO: 21 - DNA, 4474 bp, Artificial Sequence
  Synthetic Polynucleotide
gtgtttgttaattatactgtcgctgtaatatcttctcttccattatcaccggtcattccttgcaggggcggtagtacccggagaccctgaacttttctttttttttttgcgaaattaaaaagttcattttcaattcgacaatgagatctacaagccattgttttatgttgatgagagccagcttaaagagttctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaatctagaatgaaacttatgaatccatctatgaaggcatacgttttctttatcttaagctacttctctttactcgttagctcagctgcggtgccaacctctgccgccgtacaagttgagtcatacaattatgacggtaccactttttcaggtagaatattcgtcaaaaacattgcctactcaaaggtcgtaacagttatctactccgatggatcagataactggaacaataacaacaacaaagtttctgcagcttactcagaagcaatttctgggtctaactacgaatactggacattctccgcaaagttatccggaattaaacagttttatgtcaaatacgaagtttctggttcaacatattacgacaacaacggtaccaaaaactaccaagtccaagcaacctcagcgacatctacaacagctactgcaaccacaactacagctactggcacaacaactacttctacaggtccaactagtactgcatccgtatcattccctaccggtaactcaacaatttcttcctggataaaaaatcaagaggaaatcagccgttttgctatgttgagaaatatcaatccacctgggtctgccacagggttcatagccgcatctctgtccacagccggcccagattactattactcttggactagagattcagcactaacagctaatgtgatcgcttacgaatacaacacaacattcactggaaacaccacccttcttaagtacttgaaagattacgttacattttctgtcaaaagccaatctgtatctaccgtttgtaactgtctgggagaaccaaagttcaacgctgatggtagttcttttacaggtccatggggcagaccacaaaacgacggaccagcagagagagctgttacttttatgttgattgctgacagctacttgactcaaactaaggacgcatcctacgttaccggtacattaaagccagcaatcttcaaagatcttgattacgtagtttctgtttggtctaacggttgctacgatttatgggaagaggttaatggtgttcatttctatactctcatggtcatgagaaagggtttgatcttaggtgccgacttcgctgctagaaatggtgactctagtagagcttcaacctacaagcaaactgcatcaacaatggaatcaaagatcagttctttttggtcagattctaacaactacgtccaagtttctcaatcagttaccgccggagtgtcaaaaaagggactagatgttagtacactattggcggccaacattggtagtctgcctgatggctttttcactccaggctccgaaaagatattggctacagcagtggcgttagaaaatgcattcgcatccttgtacccaattaactctaacctaccttcttacttgggtaactcaattggaagatatcctgaggatacatacaacggtaatggcaactctcaggggaatccatggttccttgccgtcaacgcatacgcagaactttactacagagctattaaggaatggattagtaatggcaaggtgacagtatccaatatctcactacctttcttcaaaaagtttgattcttccgccacttctggaaagacatacactgctggtacatcagatttcaataacttggctcagaacattgctttaggcgccgatagattcctgtctactgttaagttccacgcatacactaacgggagtctatcagaagagtacgatagatctaccggtatgagtactggggctcgtgatttaacatggtcccatgcttcattgatcacagtggcgtacgcaaaggccggtagtcctgcagcttagttaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagaccgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtctccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataagagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggc

SEQ ID NO: 22 - DNA, 1878 bp, Artificial Sequence
  Synthetic Polynucleotide
gaagattaccgaagcctctgctgctgatttggtctccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataagagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatcccaatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttcggtcatgacttgatttcccacatctatgctgctgatggttctgccgacgtcatgagagacatttctgcctctggtgagccagccatccctaacattaaggacttgttgaacccaaatattaaggctgttaacatgaacgaattgtgggacactcatttacaaaagtggaactatcaaatggaatacttggaaaagtggcgtgaagctgaagaaaaagctggtaaggaattggacgctattatcgctccaattactcctaccgccgctgtcagacacgatcaattcagatactacggttacgcctccgttattaacttattggatttcacctctgttgtcgtcccagtcactttcgctgataagaatattgataagaagaacgaatcttttaaagctgtttccgaattggatgctttggttcaagaagaatacgacccagaggcttatcacggtgctcctgttgctgttcaagttattggtagaagattgtccgaagagagaactttggctatcgccgaagaagtcggtaaattgttgggtaacgtcgtcactccataaggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgataacttcgtataatgtatgctatacgaagttatgcggccgccagcacgcagcacgctgtatttacgtatttaattttatatatttgtgcatacactactagggaagacttgaaaaaaacctaggaaatgaaaaaacgacacaggaagtcccgtatttactattttttccttccttttgatggggcagggcggaaatagaggataggataagcctactgc

SEQ ID NO: 23 - DNA, 3921 bp, Artificial Sequence
  Synthetic Polynucleotide
gcccgaaagagttatcgttactccgattattttgtacagctgatgggaccttgccgtcttcatttttttttttttcacctatagagccgggcagagctgcccggctcaactaagggccggaaaaaaaacggaaaaaagaaagccaagcgtgtagacgtagtataacagtatatctgacacgcacgtgatgaccacgtaatcgcatcgcccctcacatctcacctctcaccgctgactcagcttcactaaaaaggaaaatatatactctttcccaggcaaggtgacagcggtccccgtctcctccacaaaggcctctcctggggtttgagcaagtctaagtttacgtagcataaaaattctcggattgcgtcaaataataaaaaaagtaactccacttctacttctacatcggaaaaacattccattcacatatcgtctttggcctatcttgttttgtccttggtagatcaggtcagtacaaacgcaacacgctcgaggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgacaacatcaaatacctacaaattctatctaaacggtgaatggagagaatcttcctctggagaaactattgagataccatcaccatacttacatgaagtgatcggacaggttcaagcaatcactagaggagaggttgacgaagcgattgctagcgctaaggaagcacagaaatcttgggctgaggcatctctacaagatagagctaagtacttgtacaaatgggcagatgaattggtaaacatgcaagacgaaatcgccgatatcatcatgaaggaagtgggcaagggttacaaagacgctaaaaaggaggttgttagaaccgccgatttcatcagatacaccattgaagaggcactccatatgcacggtgaatccatgatgggcgattcatttcctggtggaacaaaatctaagctagcaataatccaaagagcgcctctgggtgtagtcttagccatcgctccattcaattaccctgtaaacctttctgctgcaaaattggcaccagccttaattatgggtaacgctgtgatattcaagccagcaactcagggtgctatttccggcatcaaaatggttgaagctttgcataaggctggtttgccaaagggtttggttaacgttgccacaggtagaggtagcgtcataggcgattatttggtcgaacacgaagggataaacatggtttccttcaccggtggcactaacactggtaagcatttagcaaaaaaggcctcaatgattccattagtcttggaacttggtggcaaagatccaggcatcgttcgtgaagatgcagacctacaagatgctgcgaatcatatcgtatctggtgcgttcagttactcagggcagagatgtacagccattaagagagtccttgttcatgaaaatgttgctgatgaactggtatcattggttaaggaacaagtggcaaagctttctgtgggatcaccagagcaagattcaacaattgttcctctgattgacgataagtccgctgattttgttcagggtttagtggacgatgcagtcgaaaagggcgctacaattgtcattgggaacaagagagaacgtaacctaatctacccaacattgattgatcacgtcacagaggaaatgaaagttgcctgggaggaaccattcggtcctattcttccaattattagagttagtagcgacgagcaagctattgaaattgcaaataagagtgagttcggattacaagcttctgtgtttaccaaagacataaacaaggcattcgcaatcgcaaataagattgagactggttcagtgcaaatcaacggtagaacagagagaggaccagatcactttccttttatcggggttaagggatctgggatgggtgcccaaggcatcagaaagtctttggaatctatgactagagaaaaagttactgtcttaaatctcgtatgattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacgcggccgcggc

SEQ ID NO: 24 - DNA, 1130 bp, Saccharomyces cerevisiae
gctccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatctcgaggtactttagaatatctatattcaagtacgtggcgcgcatatgtttgagtgtgcacacaataaaggtttttagatattttgcggcgtcctaagaaaataaggggtttctagaaaaataacaatagcaaacaaagttccttacgatgatttcagatgtgaacagcatgg

SEQ ID NO: 25 - DNA, 4306 bp, Artificial Sequence
  Synthetic Polynucleotide
gcccgaaagagttatcgttactccgattattttgtacagctgatgggaccttgccgtcttcatttttttttttttcacctatagagccgggcagagctgcccggctcaactaagggccggaaaaaaaacggaaaaaagaaagccaagcgtgtagacgtagtataacagtatatctgacacgcacgtgatgaccacgtaatcgcatcgcccctcacatctcacctctcaccgctgactcagcttcactaaaaaggaaaatatatactctttcccaggcaaggtgacagcggtccccgtctcctccacaaaggcctctcctggggtttgagcaagtctaagtttacgtagcataaaaattctcggattgcgtcaaataataaaaaaagtaactccacttctacttctacatcggaaaaacattccattcacatatcgtctttggcctatcttgttttgtccttggtagatcaggtcagtacaaacgcaacacgcctcgaggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgacaacatcaaatacctacaaattctatctaaacggtgaatggagagaatcttcctctggagaaactattgagataccatcaccatacttacatgaagtgatcggacaggttcaagcaatcactagaggagaggttgacgaagcgattgctagcgctaaggaagcacagaaatcttgggctgaggcatctctacaagatagagctaagtacttgtacaaatgggcagatgaattggtaaacatgcaagacgaaatcgccgatatcatcatgaaggaagtgggcaagggttacaaagacgctaaaaaggaggttgttagaaccgccgatttcatcagatacaccattgaagaggcactccatatgcacggtgaatccatgatgggcgattcatttcctggtggaacaaaatctaagctagcaataatccaaagagcgcctctgggtgtagtcttagccatcgctccattcaattaccctgtaaacctttctgctgcaaaattggcaccagccttaattatgggtaacgctgtgatattcaagccagcaactcagggtgctatttccggcatcaaaatggttgaagctttgcataaggctggtttgccaaagggtttggttaacgttgccacaggtagaggtagcgtcataggcgattatttggtcgaacacgaagggataaacatggtttccttcaccggtggcactaacactggtaagcatttagcaaaaaaggcctcaatgattccattagtcttggaacttggtggcaaagatccaggcatcgttcgtgaagatgcagacctacaagatgctgcgaatcatatcgtatctggtgcgttcagttactcagggcagagatgtacagccattaagagagtccttgttcatgaaaatgttgctgatgaactggtatcattggttaaggaacaagtggcaaagctttctgtgggatcaccagagcaagattcaacaattgttcctctgattgacgataagtccgctgattttgttcagggtttagtggacgatgcagtcgaaaagggcgctacaattgtcattgggaacaagagagaacgtaacctaatctacccaacattgattgatcacgtcacagaggaaatgaaagttgcctgggaggaaccattcggtcctattcttccaattattagagttagtagcgacgagcaagctattgaaattgcaaataagagtgagttcggattacaagcttctgtgtttaccaaagacataaacaaggcattcgcaatcgcaaataagattgagactggttcagtgcaaatcaacggtagaacagagagaggaccagatcactttccttttatcggggttaagggatctgggatgggtgcccaaggcatcagaaagtctttggaatctatgactagagaaaaagttactgtcttaaatctcgtatgattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagaccgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtctccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataagagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggcggccgcggca

SEQ ID NO: 26 - DNA, 1366 bp, Artificial Sequence
  Synthetic Polynucleotide
agaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttctgtcatgacttgatttcccacatctatgctgctgatggttctgccgacgtcatgagagacatttctgcctctggtgagccagccatccctaacattaaggacttgttgaacccaaatattaaggctgttaacatgaacgaattgtgggacactcatttacaaaagtggaactatcaaatggaatacttggaaaagtggcgtgaagctgaagaaaaagctggtaaggaattggacgctattatcgctccaattactcctaccgtcgctgtcagacacgatcaattcagatactacggttacgcctccgttattagcttattggatttcacctctgttgtcgtcccagtcactttcgctgataagaatattgataagaagaacgaatcttttaaagctgtttccgaattggatgctttggttcaagaagaatacgacccagaggcttatcacggtgctcctgttgctgttcaagttattggtagaagattgtccgaagagagaactttggctatcgccgaagaagtcggtaaattgttgggtaacgtcgtcactccataaggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgataacttcgtataatgtatgctatacgaagttatgtactttagaatatctatattcaagtacgtggcgcgcatatgtttgagtgtgcacacaataaaggtttttagatattttgcggcgtcctaagaaaataaggggtttctagaaaaataacaatagcaaacaaagttccttacgatgatttcagatgtgaacagcatgg

SEQ ID NO: 27 - DNA, 2447 bp, Saccharomyces cerevisiae
ctatggaataatacaatgcacacaaacaaaaggtaacatttgaaaaatggagtagagaatatattccattcccctaattttttgcgggtcttccagggctgcgaacccatcgctcaaaacaagcgcagtgtcaattaagacatcattgaactaaaacggaaaatttgcttgcgccacacaccctggtcaatcgtaccaagggatatcactctgtacgggtgggaggaaggcgcggcaattagaatgtgtgggtgcggaagctcgccgctcccatcaagagagtggaagacgtatggtctgggtgcgaagtaccaccacgtttctttttcatctcttaagtgggattcttacgaaacacgtcacagggtcaaaagaaagagaacaaaagcaatattgtaattgtctcagtccacggcaatgacatggcatggccccgaaggctttttttgtctgtcttccttgggtcttaccccgccacgcgttaatagtgagacaagcaataacttcgtatagcatacattatacgaagttatcggagacaatcatatgggagaagcaattggaagatagaaaaaaggtactcggtacataaatatatgtaattctgggtagaagatcggtctgcattggatggtggtaacgcatttttttacacacattacttgcctcgagcatcaaatggtggttattcgtggatctatatcacgtgatttgcttaagaattgtcgttcatggtgacacttttagctttgacatgattaagctcatctcaattgatgttatctaaagtcatttcaactatctaagatgtggttgtgattgggccattttgtgaaagccagtacgccagcgtcaatacactcccgtcaattagttgcaccatgtccacaaaatcatataccagtagagctgagactcatgcaagtccggttgcatcgaaacttttacgtttaatggatgaaaagaagaccaatttgtgtgcttctcttgacgttcgttcgactgatgagctattgaaactagttgaaacgttgggtccatacatttgccttttgaaaacacacgttgatatcttggatgatttcagttatgagggtactgtcgttccattgaaagcattggcagagaaatacaagttcttgatatttgaggacagaaaattcgccgatatcggtaacacagtcaaattacaatatacatcgggcgtttaccgtatcgcagaatggtctgatatcaccaacgcccacggggttactggtgctggtattgttgctggcttgaaacaaggtgcgcaagaggtcaccaaagaaccaaggggattattgatgcttgctgaattgtcttccaagggttctctagcacacggtgaatatactaagggtaccgttgatattgcaaagagtgataaagatttcgttattgggttcattgctcagaacgatatgggaggcagagaagaagggtttgattggctaatcatgaccccaggtgtaggtttagacgacaaaggcgatgcattgggtcagcagtacagaaccgtcgacgaagttgtaagtggtggatcagatatcatcattgttggcagaggacttttcgccaagggtagagatcctaaggttgaaggtgaaagatacagaaatgctggatgggaagcgtaccaaaagagaatcagcgctccccattaattatacaggaaacttaatagaacaaatcacatatttaatctaatagccacctgcattggcacggtgcaacactacttcaacttcatcctacaaaaagatcacgtgatctgttgtattgaactgaaaattttttgtttgcttctctctctctctttcattatgtgagatttaaaaaccagaaactacatcatcgaaaaagaataacttcgtatagcatacattatacgaagttatactggccgtcgttttacaaccggccgctactagtaacaaaaaacccctagccccccgtttcgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtaagctctggaaacagtcttgtgggtcttggagacagtagcgtattgtcttggtctcaaagtggaaataccttgtagagcactaccacagtcaccacactttggtctagtagccaacttcttaacgtgttgggcacgcaagataccacctggggtcttaacaaccttgattttgttagaacgggtgttgtctgtacgtagtaaagagaaaattttcccattaatgttagtaatcacttctttattatcctatgatttaagaacttgagtgggattgctccatatg

SEQ ID NO: 28 - DNA, 4158 bp, Artificial Sequence
  Synthetic Polynucleotide
tgagctccgggtgggaggaaggcgcggcaattagaatgtgtgggtgcggaagctcgccgctcccatcaagagagtggaagacgtatggtctgggtgcgaagtaccaccacgtttctttttcatctcttaagtgggattcttacgaaacacgtcacagggtcaaaagaaagagaacaaaagcaatattgtaattgtctcagtccacggcaatgacatggcatggccccgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttctcgaggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgacaacatcaaatacctacaaattctatctaaacggtgaatggagagaatcttcctctggagaaactattgagataccatcaccatacttacatgaagtgatcggacaggttcaagcaatcactagaggagaggttgacgaagcgattgctagcgctaaggaagcacagaaatcttgggctgaggcatctctacaagatagagctaagtacttgtacaaatgggcagatgaattggtaaacatgcaagacgaaatcgccgatatcatcatgaaggaagtgggcaagggttacaaagacgctaaaaaggaggttgttagaaccgccgatttcatcagatacaccattgaagaggcactccatatgcacggtgaatccatgatgggcgattcatttcctggtggaacaaaatctaagctagcaataatccaaagagcgcctctgggtgtagtcttagccatcgctccattcaattaccctgtaaacctttctgctgcaaaattggcaccagccttaattatgggtaacgctgtgatattcaagccagcaactcagggtgctatttccggcatcaaaatggttgaagctttgcataaggctggtttgccaaagggtttggttaacgttgccacaggtagaggtagcgtcataggcgattatttggtcgaacacgaagggataaacatggtttccttcaccggtggcactaacactggtaagcatttagcaaaaaaggcctcaatgattccattagtcttggaacttggtggcaaagatccaggcatcgttcgtgaagatgcagacctacaagatgctgcgaatcatatcgtatctggtgcgttcagttactcagggcagagatgtacagccattaagagagtccttgttcatgaaaatgttgctgatgaactggtatcattggttaaggaacaagtggcaaagctttctgtgggatcaccagagcaagattcaacaattgttcctctgattgacgataagtccgctgattttgttcagggtttagtggacgatgcagtcgaaaagggcgctacaattgtcattgggaacaagagagaacgtaacctaatctacccaacattgattgatcacgtcacagaggaaatgaaagttgcctgggaggaaccattcggtcctattcttccaattattagagttagtagcgacgagcaagctattgaaattgcaaataagagtgagttcggattacaagcttctgtgtttaccaaagacataaacaaggcattcgcaatcgcaaataagattgagactggttcagtgcaaatcaacggtagaacagagagaggaccagatcactttccttttatcggggttaagggatctgggatgggtgcccaaggcatcagaaagtctttggaatctatgactagagaaaaagttactgtcttaaatctcgtatgattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacgcggccgcggc

SEQ ID NO: 29 - DNA, 1127 bp, Saccharomyces cerevisiae
gctccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatctcgaggataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtc

SEQ ID NO: 30 - DNA, 4542 bp, Artificial Sequence
  Synthetic Polynucleotide
tgagctccgggtgggaggaaggcgcggcaattagaatgtgtgggtgcggaagctcgccgctcccatcaagagagtggaagacgtatggtctgggtgcgaagtaccaccacgtttctttttcatctcttaagtgggattcttacgaaacacgtcacagggtcaaaagaaagagaacaaaagcaatattgtaattgtctcagtccacggcaatgacatggcatggccccgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttctcgaggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgacaacatcaaatacctacaaattctatctaaacggtgaatggagagaatcttcctctggagaaactattgagataccatcaccatacttacatgaagtgatcggacaggttcaagcaatcactagaggagaggttgacgaagcgattgctagcgctaaggaagcacagaaatcttgggctgaggcatctctacaagatagagctaagtacttgtacaaatgggcagatgaattggtaaacatgcaagacgaaatcgccgatatcatcatgaaggaagtgggcaagggttacaaagacgctaaaaaggaggttgttagaaccgccgatttcatcagatacaccattgaagaggcactccatatgcacggtgaatccatgatgggcgattcatttcctggtggaacaaaatctaagctagcaataatccaaagagcgcctctgggtgtagtcttagccatcgctccattcaattaccctgtaaacctttctgctgcaaaattggcaccagccttaattatgggtaacgctgtgatattcaagccagcaactcagggtgctatttccggcatcaaaatggttgaagctttgcataaggctggtttgccaaagggtttggttaacgttgccacaggtagaggtagcgtcataggcgattatttggtcgaacacgaagggataaacatggtttccttcaccggtggcactaacactggtaagcatttagcaaaaaaggcctcaatgattccattagtcttggaacttggtggcaaagatccaggcatcgttcgtgaagatgcagacctacaagatgctgcgaatcatatcgtatctggtgcgttcagttactcagggcagagatgtacagccattaagagagtccttgttcatgaaaatgttgctgatgaactggtatcattggttaaggaacaagtggcaaagctttctgtgggatcaccagagcaagattcaacaattgttcctctgattgacgataagtccgctgattttgttcagggtttagtggacgatgcagtcgaaaagggcgctacaattgtcattgggaacaagagagaacgtaacctaatctacccaacattgattgatcacgtcacagaggaaatgaaagttgcctgggaggaaccattcggtcctattcttccaattattagagttagtagcgacgagcaagctattgaaattgcaaataagagtgagttcggattacaagcttctgtgtttaccaaagacataaacaaggcattcgcaatcgcaaataagattgagactggttcagtgcaaatcaacggtagaacagagagaggaccagatcactttccttttatcggggttaagggatctgggatgggtgcccaaggcatcagaaagtctttggaatctatgactagagaaaaagttactgtcttaaatctcgtatgattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagaccgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtctccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataagagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggcggccgcggca

SEQ ID NO: 31 - DNA, 1363 bp, Artificial Sequence
  Synthetic Polynucleotide
agaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttctgtcatgacttgatttcccacatctatgctgctgatggttctgccgacgtcatgagagacatttctgcctctggtgagccagccatccctaacattaaggacttgttgaacccaaatattaaggctgttaacatgaacgaattgtgggacactcatttacaaaagtggaactatcaaatggaatacttggaaaagtggcgtgaagctgaagaaaaagctggtaaggaattggacgctattatcgctccaattactcctaccgtcgctgtcagacacgatcaattcagatactacggttacgcctccgttattagcttattggatttcacctctgttgtcgtcccagtcactttcgctgataagaatattgataagaagaacgaatcttttaaagctgtttccgaattggatgctttggttcaagaagaatacgacccagaggcttatcacggtgctcctgttgctgttcaagttattggtagaagattgtccgaagagagaactttggctatcgccgaagaagtcggtaaattgttgggtaacgtcgtcactccataaggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgataacttcgtataatgtatgctatacgaagttatgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtc

SEQ ID NO: 32 - DNA, 4825 bp, Artificial Sequence
  Synthetic Polynucleotide
ccgggctaattgaggggtgtcgcccttattcgactcggggtgagctcacccaccttcatccaccatatccgaagttataggggaaatataatcgtcgatgtcattgatcacgtcgttatagttgatattgtcgttagagtccagttgttgggcggatctcgtcaggtgcggatcatgaaagatattaccggcaccacctctaccaattgcaaaacgaggaaccttttcctggttgctaccgttattattgttgtttgctactgtctttgaattggatttcaatggaagaagtacgggagacggcttggacatagatttatggatgttgccagctccgcctctgccagtggagaccttgtactcttgtacacgtgcctggttctccatctcgttttgtgggttgaacgtagccatactaacttggtcttacgctactgctgctgctaacgctgctgctgcttttgctcatatgcttccattgaccgtcattagtatcagcgtcagcctttttgacataagccaccgctctgtcagggtaaccctatgaaacatttcaaaacgttataaaggaactcgtctggttacaacaaggaaatatcactacaaacagctgtccgtacggctcctcaactctctcaatgttgttcgcctggtcacacacagcatagtttcgtcattcggcgccgacggtcgctgtctcttggagccttcaagctcttgtcaacccaggtccgttgtgccgataaaagtaacagcagacccccacgcccgcatcccactctcttctccgaccacctccctcgaagttcttccctgccaatcccacgtcgatccagcgtagttggccccaactggtgcagtaataaccgcttagcgattttgcactcggaactacatatgtatatatatatgtgtgtgtgtgtgtgggctggaaagatttcttgagcttccgtgttatagtgcaatttaaatattgtacatcattccgatccagctggaaacaaaagcaagaacactcgaggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgacaacatcaaatacctacaaattctatctaaacggtgaatggagagaatcttcctctggagaaactattgagataccatcaccatacttacatgaagtgatcggacaggttcaagcaatcactagaggagaggttgacgaagcgattgctagcgctaaggaagcacagaaatcttgggctgaggcatctctacaagatagagctaagtacttgtacaaatgggcagatgaattggtaaacatgcaagacgaaatcgccgatatcatcatgaaggaagtgggcaagggttacaaagacgctaaaaaggaggttgttagaaccgccgatttcatcagatacaccattgaagaggcactccatatgcacggtgaatccatgatgggcgattcatttcctggtggaacaaaatctaagctagcaataatccaaagagcgcctctgggtgtagtcttagccatcgctccattcaattaccctgtaaacctttctgctgcaaaattggcaccagccttaattatgggtaacgctgtgatattcaagccagcaactcagggtgctatttccggcatcaaaatggttgaagctttgcataaggctggtttgccaaagggtttggttaacgttgccacaggtagaggtagcgtcataggcgattatttggtcgaacacgaagggataaacatggtttccttcaccggtggcactaacactggtaagcatttagcaaaaaaggcctcaatgattccattagtcttggaacttggtggcaaagatccaggcatcgttcgtgaagatgcagacctacaagatgctgcgaatcatatcgtatctggtgcgttcagttactcagggcagagatgtacagccattaagagagtccttgttcatgaaaatgttgctgatgaactggtatcattggttaaggaacaagtggcaaagctttctgtgggatcaccagagcaagattcaacaattgttcctctgattgacgataagtccgctgattttgttcagggtttagtggacgatgcagtcgaaaagggcgctacaattgtcattgggaacaagagagaacgtaacctaatctacccaacattgattgatcacgtcacagaggaaatgaaagttgcctgggaggaaccattcggtcctattcttccaattattagagttagtagcgacgagcaagctattgaaattgcaaataagagtgagttcggattacaagcttctgtgtttaccaaagacataaacaaggcattcgcaatcgcaaataagattgagactggttcagtgcaaatcaacggtagaacagagagaggaccagatcactttccttttatcggggttaagggatctgggatgggtgcccaaggcatcagaaagtctttggaatctatgactagagaaaaagttactgtcttaaatctcgtatgattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagaccgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtctccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataagagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggcggccgc

SEQ ID NO: 33 - DNA, 1029 bp, Saccharomyces cerevisiae
catggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatctcgaggtatctgattttcctttttcacccttcacgtaaacctgaaatatatttcatgtaatatatatagttcatc

SEQ ID NO: 34 - DNA, 4442 bp, Artificial Sequence
  Synthetic Polynucleotide
ccgggctaattgaggggtgtcgcccttattcgactcggggtgagctcacccaccttcatccaccatatccgaagttataggggaaatataatcgtcgatgtcattgatcacgtcgttatagttgatattgtcgttagagtccagttgttgggcggatctcgtcaggtgcggatcatgaaagatattaccggcaccacctctaccaattgcaaaacgaggaaccttttcctggttgctaccgttattattgttgtttgctactgtctttgaattggatttcaatggaagaagtacgggagacggcttggacatagatttatggatgttgccagctccgcctctgccagtggagaccttgtactcttgtacacgtgcctggttctccatctcgttttgtgggttgaacgtagccatactaacttggtcttacgctactgctgctgctaacgctgctgctgcttttgctcatatgcttccattgaccgtcattagtatcagcgtcagcctttttgacataagccaccgctctgtcagggtaaccctatgaaacatttcaaaacgttataaaggaactcgtctggttacaacaaggaaatatcactacaaacagctgtccgtacggctcctcaactctctcaatgttgttcgcctggtcacacacagcatagtttcgtcattcggcgccgacggtcgctgtctcttggagccttcaagctcttgtcaacccaggtccgttgtgccgataaaagtaacagcagacccccacgcccgcatcccactctcttctccgaccacctccctcgaagttcttccctgccaatcccacgtcgatccagcgtagttggccccaactggtgcagtaataaccgcttagcgattttgcactcggaactacatatgtatatatatatgtgtgtgtgtgtgtgggctggaaagatttcttgagcttccgtgttatagtgcaatttaaatattgtacatcattccgatccagctggaaacaaaagcaagaacactcgaggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgacaacatcaaatacctacaaattctatctaaacggtgaatggagagaatcttcctctggagaaactattgagataccatcaccatacttacatgaagtgatcggacaggttcaagcaatcactagaggagaggttgacgaagcgattgctagcgctaaggaagcacagaaatcttgggctgaggcatctctacaagatagagctaagtacttgtacaaatgggcagatgaattggtaaacatgcaagacgaaatcgccgatatcatcatgaaggaagtgggcaagggttacaaagacgctaaaaaggaggttgttagaaccgccgatttcatcagatacaccattgaagaggcactccatatgcacggtgaatccatgatgggcgattcatttcctggtggaacaaaatctaagctagcaataatccaaagagcgcctctgggtgtagtcttagccatcgctccattcaattaccctgtaaacctttctgctgcaaaattggcaccagccttaattatgggtaacgctgtgatattcaagccagcaactcagggtgctatttccggcatcaaaatggttgaagctttgcataaggctggtttgccaaagggtttggttaacgttgccacaggtagaggtagcgtcataggcgattatttggtcgaacacgaagggataaacatggtttccttcaccggtggcactaacactggtaagcatttagcaaaaaaggcctcaatgattccattagtcttggaacttggtggcaaagatccaggcatcgttcgtgaagatgcagacctacaagatgctgcgaatcatatcgtatctggtgcgttcagttactcagggcagagatgtacagccattaagagagtccttgttcatgaaaatgttgctgatgaactggtatcattggttaaggaacaagtggcaaagctttctgtgggatcaccagagcaagattcaacaattgttcctctgattgacgataagtccgctgattttgttcagggtttagtggacgatgcagtcgaaaagggcgctacaattgtcattgggaacaagagagaacgtaacctaatctacccaacattgattgatcacgtcacagaggaaatgaaagttgcctgggaggaaccattcggtcctattcttccaattattagagttagtagcgacgagcaagctattgaaattgcaaataagagtgagttcggattacaagcttctgtgtttaccaaagacataaacaaggcattcgcaatcgcaaataagattgagactggttcagtgcaaatcaacggtagaacagagagaggaccagatcactttccttttatcggggttaagggatctgggatgggtgcccaaggcatcagaaagtctttggaatctatgactagagaaaaagttactgtcttaaatctcgtatgattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacgcggccgc

SEQ ID NO: 35 - DNA, 1447 bp, Artificial Sequence
  Synthetic Polynucleotide
gcggccgcgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatcccaatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttcggtcatgacttgatttcccacatctatgctgctgatggttctgccgacgtcatgagagacatttctgcctctggtgagccagccatccctaacattaaggacttgttgaacccaaatattaaggctgttaacatgaacgaattgtgggacactcatttacaaaagtggaactatcaaatggaatacttggaaaagtggcgtgaagctgaagaaaaagctggtaaggaattggacgctattatcgctccaattactcctaccgccgctgtcagacacgatcaattcagatactacggttacgcctccgttattaacttattggatttcacctctgttgtcgtcccagtcactttcgctgataagaatattgataagaagaacgaatcttttaaagctgtttccgaattggatgctttggttcaagaagaatacgacccagaggcttatcacggtgctcctgttgctgttcaagttattggtagaagattgtccgaagagagaactttggctatcgccgaagaagtcggtaaattgttgggtaacgtcgtcactccataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacataacttcgtataatgtatgctatacgaagttatctcgaggtatctgattttcctttttcacccttcacgtaaacctgaaatatatttcatgtaatatatatagttcatc

SEQ ID NO: 36 - DNA, 3579 bp, Artificial Sequence
  Synthetic Polynucleotide
gaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtgggagaatactcccaatgctcttatctatgggacttccgggaaacacagtaccgatacttcccaattcgtcttcagagctcattgtttgtttgaagagactaatcaaagaatcgttttctcaaaaaatttaatatcttaactgatagtttgatcaaaggcggccgccgcgctgcggatatttctaaggcatggtcgtgcggagctacaataatacgattgaattatagctacatagtgtacaaaagcgggtatatactttcatatgtgatcagtttttggtggcagaggagcttgttgagcttgatgatgtactgtataattcatggacgaaattttcaccccagaaggcagaagtgtatttagagatgtatttgtaaagtttttcccagttaacttctttctttacatcgggcaaagtcaaggcctcgttgatggcatcagaaagatcatcggtgttccaaggatttacaataatagcaccattcaaggattgtgcggcacctgtgaactcactcaggattaaggaacctttcttttcttcttggcaagcaatatattcgtaggaaaccaagttcataccatcacgggtggacgagaccaaacagacatcgctcacagcatataacgaaatcagctcttcaaatggtatagacttgtgcatgaaatggatggggacgaattccacagtaccgaactgaccgttgattctaccgaccaactcattgaccacagatcttaaatattggtactcttccacatctccacgacttggcactgcaacctgtaccagaacaaccttgcccctccattctggatgctcgttcagaaacacttccatggcgtgcaacttctgaggcacacctttgatgtaatccagcctgtcgacaccaactatgatcttgcagcccttgaaagtttccttcaattgttggattctcttttgtacggattcctttttcaacccatcggtgaacttgtccacgtcgataccgatagggaaggcccctacgttaacgaatctgccctggtattccaccccattaggcaatgtgttcacgttaagcactctttgcacggaagacaagaaatgtcttgcataatcgtatgtgtggaacccgactaaatcacaactcaaaacacccttcaaaatctcttgtctgacaggtaagattctgtaaatttcactcgaagggaatggtgtgtgcaggaaccacccgaccttaacgttttgcagttgcttctcgtgaatcttgactctcaacatttccggaaccaacatcaaatggtaatcatgcacccagattaaatcgttatggttcatagtcttagcaatctcgttggtgaacgtctggtttgcctcgttgtatgccaaccacgcattctcgtcgaaattgatctcaccaggatggtaatggaataacggccatagaatagaattactgaacccgttgtagtgtaagtctgcgatttcatcgctcaggaagatgggtacggcattaaacttttccagcaagtccttcctcacctgatccttctcatcgtcaggaatctctagcccaggccatccgaaccacttgaaagtgtacgtcttcttcaacccttccaacgccgtgaccagccctccggacgacattgcgtactcgtactgtcccgtactgctgtttttagtgattgtcacgggaagcctgttggacaccacaataatgttaccccctgaagacgaggtcagttgcgccttagcgttatccgtagtcattgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaaatcacgagcgacggtaatttctttctcgataagaggccacgtgctttatgagggtaacatcaattcaagaaggagggaaacacttcctttttctggccctgataatagtatgagggtgaagccaaaataaaggattcgcgcccaaatcggcatctttaaatgcaggtatgcgatagttcctcactctttccttactcacgagctcataacttcgtatagcatacattatacgaagttatttaattaaatttaaactgtgaggaccttaatacattcagacacttcggcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggatgccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaggcgcgccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctctaccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgc

SEQ ID NO: 37 - DNA, 4848 bp, Artificial Sequence
  Synthetic Polynucleotide
gacaatctatcgattgtatgggaagcccgatgcgccagagttgtttctgaaacatggcaaaggtagcgttgccaatgatgttacagatgagatggtcagactaaactggctgacggaatttatgcctctaccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaacctagggcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgataacttcgtatagcatacattatacgaagttatttaattaacccgggatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaacaaacaaaatgaccaccactgcccaagacaattctccaaagaagagacagcgtatcatcaattgtgtcacgcagctgccctacaaaatccaattgggagaaagcaacgatgactggaaaatatctgctactacaggtaacagcgcattatattcctctctagaataccttcaatttgattctaccgagtacgagcaacacgttgttggttggaccggcgaaataacaagaaccgaacgcaacctgtttactagagaagcgaaagagaaaccacaggatctggacgatgacccactatatttaacaaaagagcagatcaatgggttgactactactctacaagatcatatgaaatctgataaagaggcaaagaccgatactactcaaacagctcccgttaccaataacgttcatcccgtttggctacttagaaaaaaccagagtagatggagaaattacgcggaaaaagtaatttggccaaccttccactacatcttgaatccttcaaatgaaggtgagcaagaaaaaaactggtggtacgactacgtcaagtttaacgaagcttatgcacaaaaaatcggggaagtttacaggaagggtgacatcatctggatccatgactactacctactgctattgcctcaactactgagaatgaaatttaacgacgaatctatcattattggttatttccatcatgccccatggcctagtaatgaatattttcgctgtttgccacgtagaaaacaaatcttagatggtcttgttggggccaatagaatttgtttccaaaatgaatctttctcccgtcattttgtatcgagttgtaaaagattactcgacgcaaccgccaagaaatctaaaaactcttccgatagtgatcaatatcaagtgtctgtgtacggtggtgacgtactcgtagattctttgcctataggtgttaacacaactcaaatactgaaagatgctttcacgaaggatatagattccaaggttctttccatcaagcaagcttatcaaaacaaaaaaattattattggtagagatcgtctggattccgtcagaggcgtcgttcaaaaattaagagcttttgaaactttcttggccatgtatccagaatggcgagatcaagtggtattgatccaggtcagcagtcctactgctaacagaaattccccccaaactatcagattggaacaacaagtcaacgagttggttaattccataaattctgaatatggtaatttgaatttttctcccgtccagcattattatatgagaatccctaaagatgtatacttgtccttactaagagttgcagacttatgtttaatcacaagtgttagagacggtatgaataccactgctttggaatacgtcactgtgaaatctcacatgtcgaactttttatgctacggaaatccattgattttaagtgagttttctggctctagtaacgtattgaaagatgccattgtcgttaacccatgggattcggtggccgtggctaaatctattaacatggctttgaaattggacaaggaagaaaagtccaatttagaatcaaaattatggaaagaagttcctacaattcaagattggactaataagtttttgagttcattaaaggaaaaggcgtcatctgatgatgatgtggaaaggaaaatgactccagcacttaatagacctgttcttttagaaaactacaagcaggctaagcgtagattattcctttttgattacgatggtactttgaccccaattgtcaaagacccagctgcagctattccatcggcaagactttatacaattctacaaaaattatgtgccgatcctcataatcaaatctggattatttctggtcgtgaccagaagtttttgaacaagtggttaggcggtaaacttcctcaactgggtctaagtgcggagcatggatgtttcatgaaagatgtttcttgccaagattgggtcaatttgaccgaaaaagttgatatgtcttggcaagtacgcgtcaatgaagtgatggaagaatttaccacaaggaccccaggttcattcatcgaaagaaagaaagtcgctctaacttggcattatagacgtaccgttccagaattgggtgaattccacgccaaagaactgaaagaaaaattgttatcatttactgatgacttcgatttagaggtcatggatggtaaagcaaacattgaagttcgtccaagattcgtcaacaaaggtgaaatagtcaagagactagtctggcatcaacatggcaaaccacaggacatgttgaagggaatcagtgaaaaactacctaaggatgaaatgcctgattttgtattatgtctgggtgatgacttcactgacgaagacatgtttagacagttgaataccattgaaacttgttggaaagaaaaatatcctgaccaaaaaaatcaatggggcaactacggattctatcctgtcactgtgggatctgcatccaagaaaactgtcgcaaaggctcatttaaccgatcctcagcaagtcctggagactttaggtttacttgttggtgatgtctctctcttccaaagtgctggtacggtcgacctggattccagaggtcatgtcaagaatagtgagagcagtttgaaatcaaagctagcatctaaagcttatgttatgaaaagatcggcttcttacaccggcgcaaaggtttgaacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggactcgaggcgggcctatacaggaagtagtatttgtaaaagtaaaccatgttgctagtacgaacgacttccctgaatgtgtcaaggatgccagtgccatgcctcgccagaggaataggcatcctcaagggcaaatatagactagcgaacctgatgaatgcccaaccctcagtgagacatgtgtcgagcgagatccagcaaaaggatcagcaggcaggagagtcaaacaccgccaccgatactggtgttattcacaaatcagatgaagaaactctgatatatttcgataatgtttacgctagaaccacctcggtttggaatccaacactgtggtacaatctcctgctaagaaaccagtcacgggatgcagtgaggg

SEQ ID NO: 38 - protein, 515 aa, Saccharomycopsis fibuligera
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 39 - protein, 599 aa, Rhizopus oryzae
MKFISTFLTFILAAVSVTAGASIPSSASVQLDSYNYDGSTFSGKIYVKNIAYSKKVTVVYADGSDNWNNNGNTIAASFSGPISGSNYEYWTFSASVKGIKEFYIKYEVSGKTYYDNNNSANYQVSTSKPTTTTAATTTTTAPSTSTTTRPSSSEPATFPTGNSTISSWIKKQEDISRFAMLRNINPPGSATGFIAASLSTAGPDYYYAWTRDAALTSNVIVYEYNTTLSGNKTILNVLKDYVTFSVKTQSTSTVCNCLGEPKFNPDGSGYTGAWGRPQNDGPAERATTFVLFADSYLTQTKDASYVTGTLKPAIFKDLDYVVNVWSNGCFDLWEEVNGVHFYTLMVMRKGLLLGADFAKRNGDSTRASTYSSTASTIANKISSFWVSSNNWVQVSQSVTGGVSKKGLDVSTLLAANLGSVDDGFFTPGSEKILATAVAVEDSFASLYPINKNLPSYLGNAIGRYPEDTYNGNGNSQGNPWFLAVTGYAELYYRAIKEWISNGGVTVSSISLPFFKKFDSSATSGKKYTVGTSDFNNLAQNIALAADRFLSTVQLHAPNNGSLAEEFDRTTGFSTGARDLTWSHASLITASYAKAGAPAA

SEQ ID NO: 40 - protein, 604 aa, Rhizopus delemar
MQLFNLPLKVSFFLVLSYFSLLVSAASIPSSASVQLDSYNYDGSTFSGKIYVKNIAYSKKVTVIYADGSDNWNNNGNTIAASYSAPISGSNYEYWTFSASINGIKEFYIKYEVSGKTYYDNNNSANYQVSTSKPTTTTATATTTTAPSTSTTTPPSSSEPATFPTGNSTISSWIKKQEGISRFAMLRNINPPGSATGFIAASLSTAGPDYYYAWTRDAALTSNVIVYEYNTTLSGNKTILNVLKDYVTFSVKTQSTSTVCNCLGEPKFNPDGSGYTGAWGRPQNDGPAERATTFILFADSYLTQTKDASYVTGTLKPAIFKDLDYVVNVWSNGCFDLWEEVNGVHFYTLMVMRKGLLLGADFAKRNGDSTRASTYSSTASTIANKISSFWVSSNNWIQVSQSVTGGVSKKGLDVSTLLAANLGSVDDGFFTPGSEKILATAVAVEDSFASLYPINKNLPSYLGNSIGRYPEDTYNGNGNSQGNPWFLAVTGYAELYYRAIKEWIGNGGVTVSSISLPFFKKFDSSATSGKKYTVGTSDFNNLAQNIALAADRFLSTVQLHAHNNGSLAEEFDRTTGLSTGARDLTWSHASLITASYAKAGAPAA

SEQ ID NO: 41 - protein, 605 aa, Rhizopus microsporus
MKLMNPSMKAYVFFILSYFSLLVSSAAVPTSAAVQVESYNYDGTTFSGRIFVKNIAYSKVVTVIYSDGSDNWNNNNNKVSAAYSEAISGSNYEYWTFSAKLSGIKQFYVKYEVSGSTYYDNNGTKNYQVQATSATSTTATATTTTATGTTTTSTGPTSTASVSFPTGNSTISSWIKNQEEISRFAMLRNINPPGSATGFIAASLSTAGPDYYYSWTRDSALTANVIAYEYNTTFTGNTTLLKYLKDYVTFSVKSQSVSTVCNCLGEPKFNADGSSFTGPWGRPQNDGPAERAVTFMLIADSYLTQTKDASYVTGTLKPAIFKDLDYVVSVWSNGCYDLWEEVNGVHFYTLMVMRKGLILGADFAARNGDSSRASTYKQTASTMESKISSFWSDSNNYVQVSQSVTAGVSKKGLDVSTLLAANIGSLPDGFFTPGSEKILATAVALENAFASLYPINSNLPSYLGNSIGRYPEDTYNGNGNSQGNPWFLAVNAYAELYYRAIKEWISNGKVTVSNISLPFFKKFDSSATSGKTYTAGTSDFNNLAQNIALGADRFLSTVKFHAYTNGSLSEEYDRSTGMSTGARDLTWSHASLITVAYAKAGSPAA

SEQ ID NO: 42 - protein, 479 aa, Bacillus cereus
MTTSNTYKFYLNGEWRESSSGETIEIPSPYLHEVIGQVQAITRGEVDEAIASAKEAQKSWAEASLQDRAKYLYKWADELVNMQDEIADIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSVIGDYLVEHEGINMVSFTGGTNTGKHLAKKASMIPLVLELGGKDPGIVREDADLQDAANHIVSGAFSYSGQRCTAIKRVLVHENVADELVSLVKEQVAKLSVGSPEQDSTIVPLIDDKSADFVQGLVDDAVEKGATIVIGNKRERNLIYPTLIDHVTEEMKVAWEEPFGPILPIIRVSSDEQAIEIANKSEFGLQASVFTKDINKAFAIANKIETGSVQINGRTERGPDHFPFIGVKGSGMGAQGIRKSLESMTREKVTVLNLV

SEQ ID NO: 43 - protein, 495 aa, Saccharomyces cerevisiae
MTTDNAKAQLTSSSGGNIIVVSNRLPVTITKNSSTGQYEYAMSSGGLVTALEGLKKTYTFKWFGWPGLEIPDDEKDQVRKDLLEKFNAVPIFLSDEIADLHYNGFSNSILWPLFHYHPGEINFDENAWLAYNEANQTFTNEIAKTMNHNDLIWVHDYHLMLVPEMLRVKIHEKQLQNVKVGWFLHTPFPSSEIYRILPVRQEILKGVLSCDLVGFHTYDYARHFLSSVQRVLNVNTLPNGVEYQGRFVNVGAFPIGIDVDKFTDGLKKESVQKRIQQLKETFKGCKIIVGVDRLDYIKGVPQKLHAMEVFLNEHPEWRGKVVLVQVAVPSRGDVEEYQYLRSVVNELVGRINGQFGTVEFVPIHFMHKSIPFEELISLYAVSDVCLVSSTRDGMNLVSYEYIACQEEKKGSLILSEFTGAAQSLNGAIIVNPWNTDDLSDAINEALTLPDVKKEVNWEKLYKYISKYTSAFWGENFVHELYSTSSSSTSSSATKN

SEQ ID NO: 44 - protein, 896 aa, Saccharomyces cerevisiae
MTTTAQDNSPKKRQRIINCVTQLPYKIQLGESNDDWKISATTGNSALFSSLEYLQFDSTEYEQHVVGWTGEITRTERNLFTREAKEKPQDLDDDPLYLTKEQINGLTTTLQDHMKSDKEAKTDTTQTAPVTNNVHPVWLLRKNQSRWRNYAEKVIWPTFHYILNPSNEGEQEKNWWYDYVKFNEAYAQKIGEVYRKGDIIWIHDYYLLLLPQLLRMKFNDESIIIGYFHHAPWPSNEYFRCLPRRKQILDGLVGANRICFQNESFSRHFVSSCKRLLDATAKKSKNSSNSDQYQVSVYGGDVLVDSLPIGVNTTQILKDAFTKDIDSKVLSIKQAYQNKKIIIGRDRLDSVRGVVQKLRAFETFLAMYPEWRDQVVLIQVSSPTANRNSPQTIRLEQQVNELVNSINSEYGNLNFSPVQHYYMRIPKDVYLSLLRVADLCLITSVRDGMNTTALEYVTVKSHMSNFLCYGNPLILSEFSGSSNVLKDAIVVNPWDSVAVAKSINMALKLDKEEKSNLESKLWKEVPTIQDWTNKFLSSLKEQASSNDDMERKMTPALNRPVLLENYKQAKRRLFLFDYDGTLTPIVKDPAAAIPSARLYTILQKLCADPHNQIWIISGRDQKFLNKWLGGKLPQLGLSAEHGCFMKDVSCQDWVNLTEKVDMSWQVRVNEVMEEFTTRTPGSFIERKKVALTWHYRRTVPELGEFHAKELKEKLLSFTDDFDLEVMDGKANIEVRPRFVNKGEIVKRLVWHQHGKPQDMLKGISEKLPKDEMPDFVLCLGDDFTDEDMFRQLNTIETCWKEKYPDQKNQWGNYGFYPVTVGSASKKTVAKAHLTDPQQVLETLGLLVGDVSLFQSAGTVDLDSRGHVKNSESSLKSKLASKAYVMKRSASYTGAKV

SEQ ID NO: 45 - DNA, 1440 bp, Bacillus cereus
atgacaacatcaaatacctacaaattctatctaaacggtgaatggagagaatcttcctctggagaaactattgagataccatcaccatacttacatgaagtgatcggacaggttcaagcaatcactagaggagaggttgacgaagcgattgctagcgctaaggaagcacagaaatcttgggctgaggcatctctacaagatagagctaagtacttgtacaaatgggcagatgaattggtaaacatgcaagacgaaatcgccgatatcatcatgaaggaagtgggcaagggttacaaagacgctaaaaaggaggttgttagaaccgccgatttcatcagatacaccattgaagaggcactccatatgcacggtgaatccatgatgggcgattcatttcctggtggaacaaaatctaagctagcaataatccaaagagcgcctctgggtgtagtcttagccatcgctccattcaattaccctgtaaacctttctgctgcaaaattggcaccagccttaattatgggtaacgctgtgatattcaagccagcaactcagggtgctatttccggcatcaaaatggttgaagctttgcataaggctggtttgccaaagggtttggttaacgttgccacaggtagaggtagcgtcataggcgattatttggtcgaacacgaagggataaacatggtttccttcaccggtggcactaacactggtaagcatttagcaaaaaaggcctcaatgattccattagtcttggaacttggtggcaaagatccaggcatcgttcgtgaagatgcagacctacaagatgctgcgaatcatatcgtatctggtgcgttcagttactcagggcagagatgtacagccattaagagagtccttgttcatgaaaatgttgctgatgaactggtatcattggttaaggaacaagtggcaaagctttctgtgggatcaccagagcaagattcaacaattgttcctctgattgacgataagtccgctgattttgttcagggtttagtggacgatgcagtcgaaaagggcgctacaattgtcattgggaacaagagagaacgtaacctaatctacccaacattgattgatcacgtcacagaggaaatgaaagttgcctgggaggaaccattcggtcctattcttccaattattagagttagtagcgacgagcaagctattgaaattgcaaataagagtgagttcggattacaagcttctgtgtttaccaaagacataaacaaggcattcgcaatcgcaaataagattgagactggttcagtgcaaatcaacggtagaacagagagaggaccagatcactttccttttatcggggttaagggatctgggatgggtgcccaaggcatcagaaagtctttggaatctatgactagagaaaaagttactgtcttaaatctcgtatga

SEQ ID NO: 46 - DNA, 1548 bp, Saccharomycopsis fibuligera
atgattagattaaccgtattcctcactgcagtttttgcagcagtcgcttcctgtgttccagttgaattggataagagaaatacaggccatttccaagcatattctggttacaccgtagctagatcaaactttactcaatggattcacgagcaaccagccgtatcatggtactatttgcttcagaatatagactatccagaaggacaattcaagtctgccaagccaggggtcgttgtggcttccccttctacatccgaacctgattacttctaccaatggactagagatactgctatcaccttcttgtcacttatcgcggaagttgaggatcattctttttcaaatactacactagccaaggtggttgaatactacatctctaatacttacacattacaaagagtttccaacccatctggtaacttcgacagtccaaatcacgacggtttgggagaaccaaagtttaatgttgatgatacagcttatactgcatcttggggtagaccacaaaatgatggcccagcgttgagagcatacgcaatttcaagataccttaacgcagtagcaaaacacaacaacggtaagttactgctcgctggacaaaacggtattccttactcttcagcttctgatatctactggaagattatcaagccagatcttcaacatgtgtcaacccattggtctacatctggttttgatttgtgggaagagaatcagggaacacatttctttactgcgttggtccagctaaaagcacttagttacggcattcctttaagtaagacctacaacgatcctggtttcactagttggctagaaaagcaaaaggatgctttaaactcttatatcaacagctctggtttcgtaaactctggcaaaaagcatatagtggagagccctcaactatcttcaagaggagggttggatagcgccacatacattgcagccttaatcacacatgatattggcgacgacgacacttacacacctttcaacgttgacaactcctatgtcttgaactcactgtattaccttctagtcgataacaaaaaccgttacaaaatcaatggtaactacaaggccggtgctgctgttggtagatacccagaggatgtttacaacggtgttgggacatcagaaggcaatccatggcaattagctacagcctacgccggccaaacattttacacactggcttacaactcattgaaaaacaaaaaaaacttagtgattgaaaagttgaactacgacctctacaattctttcatagcagatttatccaagatcgatagttcttacgcatcaaaagactccttgactttgacctacggttctgacaactacaaaaacgtcataaagtcactattacagtttggagattcattcctgaaggtcttgctcgatcacattgatgataatggacaattaacagaagagatcaatagatacacagggttccaggctggtgctgttagtttgacatggtcctctggttcattactttcagcaaaccgtgcgagaaataagttgattgaactattgtag

SEQ ID NO: 47 - DNA, 1548 bp, Saccharomycopsis fibuligera
atgatcagacttacagttttcctaacagccgttttcgccgccgttgcatcatgtgtcccagtagaattggataagagaaacaccggccatttccaagcatattcaggatacaccgttgcacgttctaatttcacacaatggattcatgagcagcctgctgtgtcctggtactacttattacaaaacattgattatcctgagggacaattcaagtcagcgaaaccaggcgttgtggttgcttctccatccacttcagaaccagactacttctaccagtggacccgtgacacagcaataactttcttatctttgatagcagaagtagaagatcactcattttcaaatacaactctagctaaggttgtcgaatactacatctctaacacatacaccctacaaagagtttctaacccatctggtaatttcgatagcccaaatcacgatggtctgggtgaaccaaagttcaacgttgacgacactgcttacactgcatcatggggcagacctcaaaacgacggtccagccttaagagcttacgcgatctcaagatatttgaacgcagttgccaagcataacaacggtaagctattgctcgcgggtcaaaatggtattccttactcatctgcatcagatatctactggaagattatcaagccagatttacaacatgtaagtactcactggagtacatctggttttgacttatgggaagagaatcaaggtacacatttctttactgcacttgtccagttaaaagctctttcatacggtatacctttgtctaagacatataacgatccaggatttacttcttggttggaaaagcagaaggatgccttgaactcttacatcaattccagcggcttcgtcaactccgggaaaaagcacattgtcgaatctcctcaattatctagtagagggggtcttgatagcgctacttacatcgctgctctaattacacatgatattggtgatgatgatacatacactccttttaacgtagataattcttatgtgctgaactctttatactatctgcttgtagacaacaaaaacagatacaagatcaacgggaactacaaagcaggagctgcagttggtagatacccagaagatgtgtacaatggagtgggaacctcagagggaaacccatggcaattggcgacagcatacgccggccaaaccttttacacactggcttacaattctctcaaaaacaaaaaaaatttggttattgagaagttgaattacgatctatacaactcctttatagctgacttaagtaagattgactcctcttacgcttctaaggattcattgacattgacctacggctcagataactacaaaaatgtcattaagtcacttttacaattcggggattctttcttgaaagtcttgttggaccatattgatgataatggtcagctaacagaggaaatcaacagatatacaggttttcaagctggcgcagtttccctcacttggagtagtggttcactcttatctgcaaacagagccagaaacaagttgatcgaattgctttag

SEQ ID NO: 48 - DNA, 1548 bp, Saccharomycopsis fibuligera
atgatcagacttactgttttcctcacagccgtttttgcagcagtagcttcttgtgttccagttgaattggataagagaaatacaggtcatttccaagcttactctggttacactgtggctagatctaacttcacacaatggattcatgaacagcctgccgtgagttggtactatttgctacaaaacattgattaccctgagggtcaattcaaatcagctaagccaggtgttgttgtcgcgagcccatcaacttctgaaccagattacttctaccaatggactagagataccgcaataaccttcttatctctaatcgcagaggtagaagatcactctttttcaaatactaccctggcaaaagtggtcgagtactacatctcaaacacatacaccttgcagagagtctcaaacccatcaggaaacttcgattctcctaatcatgacggcttaggagaaccaaagtttaatgttgacgataccgcttatactgcatcttggggtagaccacagaatgatggccctgccttacgtgcatacgccatttccagatatctcaacgctgtagcgaagcacaacaacggtaagctgcttttagctggtcaaaatgggataccatactcttccgcttcagacatttactggaagattatcaaaccagacttgcagcatgtcagtacacattggtcaacttctggttttgatttgtgggaagagaaccaaggcactcacttctttacagccttggttcaactaaaggcattgtcttacggaatccctttgtccaagacatacaatgatcctggattcactagttggctagaaaagcaaaaggatgcactgaactcatacattaacagttcaggctttgtgaactccggtaaaaagcatattgttgaaagcccacaactatctagcagaggtggtttagattctgcaacctacatagcagccttgatcacacacgacattggggatgacgatacatacacaccattcaacgtcgacaattcatacgttttgaatagcttatactacctactggtagataacaaaaacagatataagatcaatggcaactacaaggccggtgctgccgtaggaagataccctgaagatgtctacaacggagttggtacatcagaaggtaacccatggcaattagcaacagcatatgcgggccagacattttacactttggcttacaattcattgaaaaacaaaaaaaatttagtgatagaaaagcttaactatgacctttacaactctttcattgccgatttatccaagattgattcctcctacgcatcaaaggactccttgacacttacatacggttctgacaactacaaaaatgttatcaagtctctcttgcaatttggtgattctttcttgaaggttttactcgatcatatcgatgataatggtcaactaactgaggaaatcaacagatacactgggttccaagctggagctgtctctttaacatggagttcagggagtttgttatctgctaacagagcgcgtaacaaacttattgagcttctgtag

SEQ ID NO: 49 - DNA, 1548 bp, Saccharomycopsis fibuligera
atgattagattaacagtatttcttacagccgttttcgcagccgtcgcatcctgtgttccagtagaattagataagcgtaatacaggacattttcaagcttactctggctatacagttgcgagatctaactttacacaatggattcacgaacagccagcagtttcttggtactatttgctccaaaacatcgactaccctgaaggccaattcaagtctgcaaagccaggagtggtcgtcgcttctcctagtacttcagaaccagattacttctaccagtggacaagagacactgctattaccttcctgagcttaatcgctgaagttgaagatcactctttttctaatacaacactggccaaagtagttgagtactacatctctaacacttacactctacaaagagtgtcaaacccttctgggaacttcgacagcccaaaccatgatggtttgggggagccaaaattcaacgttgatgatacagcctacaccgcatcttggggtagaccacaaaacgacggaccagctttaagagcatacgcaatatctcgttaccttaatgctgttgcaaagcacaataatggaaagttgttgttggctggtcaaaacggtattccttactcttcagcatctgatatctactggaagattatcaagccagatcttcaacacgtatccacacattggtcaacctccggcttcgatttatgggaggaaaatcagggtacacatttcttcaccgctctagtgcaattgaaggctttgagttacggcattccattgtctaagacttacaacgatcctggtttcacctcatggcttgaaaagcagaaggatgccctgaatagctacatcaactcatctggttttgttaactcagggaaaaagcatatagttgaatccccacaactatcatcaagaggaggtttagactccgccacatacattgctgccttgattacacatgatattggggatgatgacacatatactccatttaacgtcgataacagttatgtccttaattccttatactatttgttggtcgataacaaaaatagatacaaaatcaacggcaactacaaggctggcgcagcggtgggtagataccctgaggatgtttacaatggtgtaggtacatctgaaggcaatccatggcaattagcgactgcttacgctggacaaactttctacacacttgcgtacaactcattgaaaaacaaaaaaaacctagtcattgaaaagttgaattacgatctgtacaactctttcatcgcagacctatcaaagattgactcatcttatgcaagtaaagattcactaactttaacctacggtagtgataactacaaaaacgttatcaagtctttactccagtttggtgattcattcttgaaggtgttgttagatcatatagacgacaatggtcaactcacagaggagataaacagatacactggttttcaagcaggagctgtttcacttacttggtcaagtggttctttgctttccgccaacagagccagaaacaagctcatcgaattactatag

SEQ ID NO: 50 - DNA, 1797 bp, Rhizopus oryzae
atgaagttcatttccactttcttgaccttcattttggctgctgtctctgtcaccgctgcatctattccatctagtgcatctgtacaattggactcctacaattacgatggttccacattttccggcaagatttatgtcaaaaacatcgcttactctaaaaaggttactgttgtgtacgcagacggttctgacaactggaacaataacggcaacactattgctgcatcattttcaggcccaatctctggatcaaattacgaatactggacattctcagcatcagtgaagggcataaaggagttctacatcaaatacgaagtttcaggtaagacatattacgacaataacaactctgcaaactaccaagtctcaacttctaaacctactacaactactgcagctacaaccacaactacagctccatcaacttctacaacaacccgtccatctagttcagagcctgccaccttccctactggtaattctaccatcagctcttggatcaaaaagcaggaagatatttccagattcgctatgcttagaaacatcaacccacctggttctgccacagggtttatcgccgcatcactctctaccgctggtccagattactactacgcgtggacaagagatgccgctttgacatctaacgttatcgtttacgaatacaacaccacattgtctgggaataagacaattctaaacgtacttaaggattacgtcacattcagtgttaagacacagtctacttcaacagtttgtaattgccttggtgaaccaaagttcaatccagacggcagtggttacacaggtgcttggggtagacctcaaaatgatggtcctgcagaaagagcgactacatttgttctgtttgccgacagctacttgactcaaactaaggatgcctcatacgtcactggtacattaaagccagcaattttcaaagatctcgattacgttgttaacgtctggagtaacggatgtttcgatttatgggaggaggtgaacggagttcatttctacacccttatggttatgagaaaagggctattgttgggggctgatttcgcgaagagaaacggtgactcaactagagcctcaacttactcttctactgcttccacaattgctaacaagatatcaagtttctgggttagctcaaacaactgggtgcaagtatcccaatctgtcacaggaggtgtaagtaaaaaggggttagacgttagcaccctgttagctgcgaatctaggatcagtcgatgatggatttttcactccaggttctgaaaagatattagctacagctgtggcagtcgaagattcctttgccagtctatacccaatcaacaaaaaccttccatcatacttggggaacgctattggaagataccctgaagatacatacaacggtaatggtaactcacaaggcaatccttggtttctggcggttaccggctacgcagagttgtactatagagcaattaaggaatggatttctaatggaggcgttacagtgtcctctatctcattgccatttttcaaaaagttcgatagctctgcaacatccggtaaaaagtacaccgtaggtacttctgacttcaacaatttagcacaaaacattgctcttgctgcagatcgtttcctatctactgtacaactccatgcaccaaacaatggttcattagcagaggaatttgatagaacaacaggtttttctaccggcgctagagatttaacatggtcccacgcctcattgataacagcatcctatgccaaagccggtgctccagctgcataa

SEQ ID NO: 51 - DNA, 1797 bp, Rhizopus oryzae
atgaagtttatctccacgtttttaacctttatcctagcagctgtcagcgtcaccgccgcatcaattccgagttcagcatctgtacaacttgactcttacaattacgatggcagcactttctcagggaaaatttatgtgaaaaacatagcatatagtaagaaggttaccgtggtatatgcagacggttctgataattggaataataatggaaacactattgccgccagtttttccggcccaatttctggttccaattacgagtattggaccttttctgcatcagtaaaaggcatcaaggaattctatattaagtacgaagtttcaggtaagacatattacgataacaataactcagcaaattatcaagtctctacatctaagcccacaacaacaactgctgctaccaccactacaaccgctccttctaccagcaccactaccagaccaagctctagtgaaccggctacctttcctaccggaaacagtaccatctcaagctggatcaaaaagcaagaggacataagtcgttttgctatgttgaggaacattaatcctccaggatccgcgaccggtttcattgcagcatcactaagtactgccgggcctgattattattatgcttggactagagacgctgcattaacatcaaacgtgattgtttatgaatataatacgaccctttccggtaataaaacgatcttgaacgtattaaaagactatgtgacctttagtgtgaagacccaatctacatctacagtgtgtaattgtttgggagaacctaaattcaatccagacggttctgggtacactggtgcctggggtagacctcaaaacgacggtccagcagaaagagcaacaacctttgttctatttgctgactcttatttaacgcaaacaaaggacgcctcatatgttacagggaccctaaaaccagcaattttcaaagacttggattatgttgttaatgtttggagcaacggatgttttgacttgtgggaggaggttaacggtgtacacttttatacattgatggtgatgagaaaagggttgctattgggagcagatttcgctaaaagaaatggtgattctacaagagcgagcacatatagtagcaccgcttcaacaatcgccaataaaatctcatctttctgggtatctagcaacaactgggtacaagtttcccaaagtgttaccggcggtgtgtccaaaaagggtttagacgttagcacacttctagctgctaatttgggtagcgttgatgacgggttttttactccaggtagtgagaagatactggcaaccgcggtggcggttgaagacagctttgcttcattgtatcctataaataaaaatctgccctcttatctgggtaatgcaattggcagatacccagaagatacctacaatggtaatggtaattcccaggggaacccatggtttttggctgttacaggctacgcagaactttattaccgtgcaatcaaggaatggatttcaaatggcggcgtcactgtcagtagtataagtttgcccttttttaagaaatttgattcctcagcaacgtctggtaaaaaatacaccgtaggtactagtgatttcaataatttggcccaaaatattgcgcttgctgctgacaggtttcttagtaccgttcagttgcacgctccaaataatggctcattggctgaagaatttgatcgtacgacaggtttctccactggtgctagggatttgacttggagtcatgcctccttaatcacagcaagctatgctaaagctggtgcacctgctgcttag

SEQ ID NO: 52 - DNA, 1815 bp, Rhizopus delemar
atgcagctgttcaacttgccattaaaggtttcattctttttggtcctatcatactttagtttgttggtgtcagccgcatctattccatcttcagcatctgtacaattagactcctacaattacgacggctctacattcagcggaaagatttacgtgaaaaatattgcgtacagcaaaaaagtaactgttatctatgccgacggatcagataactggaacaacaatggaaacactatcgctgccagttactctgcaccaatttcaggttctaactacgaatattggacattctcagcctccatcaatggcattaaggaattctacataaagtacgaagtttccggtaagacttactacgataacaacaattctgcaaactatcaagtatcaacatcaaaacctactaccaccaccgccacagctacaactacaactgcaccttcaacatctaccacaaccccaccatcttctagcgaaccagctacattcccaactggcaattctactatttctagttggatcaaaaaacaagagggtatttccagattcgcaatgttgagaaacataaatccaccaggatcagcaactggattcatcgcagcttctttgtccacagcggggccagattactactacgcatggaccagagatgctgctttgacaagtaacgttattgtttacgaatacaataccactttgtccggtaacaagactattcttaacgtcctaaaggattacgttacattctctgttaagactcagtctacatccacagtctgcaattgtttgggtgaaccaaagttcaacccagatggctctggatacacaggtgcctggggtcgtccacaaaacgatgggcctgccgagagagccactacatttatcctatttgctgactcataccttacacaaacaaaagatgcatcctacgtgactggaacattaaagcctgcaatcttcaaagacctggattacgttgtcaacgtgtggtctaacggctgtttcgatctatgggaagaggttaacggcgtgcacttctacactctaatggtcatgagaaagggtctgttgttaggtgcagattttgctaagagaaacggtgattctacacgtgcttctacctactcctcaacagcatcaactattgcgaacaagatttcttcattttgggtttcaagtaataactggatacaagtatctcaaagcgttacagggggtgtctcaaaaaagggtcttgatgtttctacattactggctgctaatcttgggtctgttgatgacggtttcttcacccctggttctgaaaagatcctcgctaccgccgtcgcggttgaggatagttttgcttcactctatcctataaacaaaaaccttccttcatacttaggaaacagtatcggtagatacccagaggatacatacaatggtaatggcaattcacagggaaatccatggttccttgctgttacagggtacgcagaactttactatagagctattaaggaatggatcggcaacggcggtgtgacagtttcctcaatctcattgccatttttcaaaaagtttgactccagcgcgacatctggtaaaaagtatactgtggggacttctgatttcaacaatttggctcaaaacattgccttagctgccgacagattcttatctaccgtacaactccatgcacataacaatggtagtttggcagaggaatttgatagaactacaggactctctacaggtgcgagagatttaacttggtcacatgcaagtttaattacagcctcttacgcaaaggctggtgctcctgctgcataa

SEQ ID NO: 53 - DNA, 1815 bp, Rhizopus delemar
atgcagttattcaacttaccacttaaggtatctttctttctagtcttatcttacttttcattgttagtatcagctgcctctataccaagttcagcatccgtacaactagattcatacaattacgacggttcaacattctcaggaaagatatacgtgaaaaatattgcttacagcaaaaaggttactgtgatttacgcagatgggtcagacaactggaataacaatggaaacacaattgctgcttcctattctgcccctatttctggatctaactacgaatactggactttttcagcgagtataaacggaattaaggaattctatatcaaatatgaagtctctggtaagacctactacgataacaacaactccgcaaactaccaagttagcacatcaaagccaaccacaacaactgctactgcgacaactacaaccgcaccaagcacttctactacaacacctcctagttcatctgagccagcaactttcccaactggtaattccactatttcttcttggatcaaaaaacaagagggtatctcaagattcgccatgcttagaaatatcaatcctccaggctctgcaacaggattcattgcagcatctttatcaactgcggggccagactactactacgcctggactagagatgcagctttgacatcaaatgtgattgtttatgaatacaacacaactttgtccggtaacaagacaatcttgaacgtcttgaaggattatgtgacattctctgtcaagactcaatctacatcaacagtttgtaactgtctcggcgaaccaaagttcaaccctgatggtagtggttacactggtgcttggggtagaccacaaaacgatggtccagcagagagagctacaactttcatcttgtttgctgactcttacctaacacaaaccaaggatgcaagctacgttactggaacactaaagcctgcaatctttaaagacctggactatgttgtaaacgtttggtcaaatggctgcttcgatctatgggaggaagtgaacggtgttcacttctacacattaatggtcatgagaaagggactcttgcttggtgcagactttgctaagagaaacggtgattctacacgtgcctccacttactcctccacagcttcaaccattgccaacaaaatctcttctttctgggtcagctcaaataactggattcaagtttctcaatcagttactggtggtgtttctaaaaagggcctggatgtgtcaaccttgcttgctgccaatttgggcagtgttgatgacgggttcttcaccccaggttctgaaaagatcctcgccaccgcagttgccgttgaagattcatttgctagtttatacccaatcaacaaaaatctaccatcataccttggaaattcaatcggtagatatccagaggatacatacaacggtaatggaaactctcagggtaacccttggtttcttgcagttacagggtacgctgaactgtactacagagcgattaaggaatggattggtaatggcggcgtaactgttagttctatttctctacctttcttcaaaaagttcgatagttctgcaacatctggtaaaaagtacacagtcggcacttccgattttaacaatttagctcagaacatagcactggcagctgatcgtttcttgagtacagtccaattgcatgcccataacaacggtagtttggctgaagagtttgatagaaccaccggtttatcaaccggcgccagagatttaacatggtcccatgcgtctttgataactgcttcttacgccaaggctggggcaccagctgcctga

SEQ ID NO: 54 - DNA, 1818 bp, Rhizopus microsporus
atgaaacttatgaatccatctatgaaggcatacgttttctttatcttaagctacttctctttactcgttagctcagctgcggtgccaacctctgccgccgtacaagttgagtcatacaattatgacggtaccactttttcaggtagaatattcgtcaaaaacattgcctactcaaaggtcgtaacagttatctactccgatggatcagataactggaacaataacaacaacaaagtttctgcagcttactcagaagcaatttctgggtctaactacgaatactggacattctccgcaaagttatccggaattaaacagttttatgtcaaatacgaagtttctggttcaacatattacgacaacaacggtaccaaaaactaccaagtccaagcaacctcagcgacatctacaacagctactgcaaccacaactacagctactggcacaacaactacttctacaggtccaactagtactgcatccgtatcattccctaccggtaactcaacaatttcttcctggataaaaaatcaagaggaaatcagccgttttgctatgttgagaaatatcaatccacctgggtctgccacagggttcatagccgcatctctgtccacagccggcccagattactattactcttggactagagattcagcactaacagctaatgtgatcgcttacgaatacaacacaacattcactggaaacaccacccttcttaagtacttgaaagattacgttacattttctgtcaaaagccaatctgtatctaccgtttgtaactgtctgggagaaccaaagttcaacgctgatggtagttcttttacaggtccatggggcagaccacaaaacgacggaccagcagagagagctgttacttttatgttgattgctgacagctacttgactcaaactaaggacgcatcctacgttaccggtacattaaagccagcaatcttcaaagatcttgattacgtagtttctgtttggtctaacggttgctacgatttatgggaagaggttaatggtgttcatttctatactctcatggtcatgagaaagggtttgatcttaggtgccgacttcgctgctagaaatggtgactctagtagagcttcaacctacaagcaaactgcatcaacaatggaatcaaagatcagttctttttggtcagattctaacaactacgtccaagtttctcaatcagttaccgccggagtgtcaaaaaagggactagatgttagtacactattggcggccaacattggtagtctgcctgatggctttttcactccaggctccgaaaagatattggctacagcagtggcgttagaaaatgcattcgcatccttgtacccaattaactctaacctaccttcttacttgggtaactcaattggaagatatcctgaggatacatacaacggtaatggcaactctcaggggaatccatggttccttgccgtcaacgcatacgcagaactttactacagagctattaaggaatggattagtaatggcaaggtgacagtatccaatatctcactacctttcttcaaaaagtttgattcttccgccacttctggaaagacatacactgctggtacatcagatttcaataacttggctcagaacattgctttaggcgccgatagattcctgtctactgttaagttccacgcatacactaacgggagtctatcagaagagtacgatagatctaccggtatgagtactggggctcgtgatttaacatggtcccatgcttcattgatcacagtggcgtacgcaaaggccggtagtcctgcagcttag

SEQ ID NO: 55 - DNA, 1488 bp, Saccharomyces cerevisiae
atgactacggataacgctaaggcgcaactgacctcgtcttcagggggtaacattattgtggtgtccaacaggcttcccgtgacaatcactaaaaacagcagtacgggacagtacgagtacgcaatgtcgtccggagggctggtcacggcgttggaagggttgaagaagacgtacactttcaagtggttcggatggcctgggctagagattcctgacgatgagaaggatcaggtgaggaaggacttgctggaaaagtttaatgccgtacccatcttcctgagcgatgaaatcgcagacttacactacaacgggttcagtaattctattctatggccgttattccattaccatcctggtgagatcaatttcgacgagaatgcgtggttggcatacaacgaggcaaaccagacgttcaccaacgagattgctaagactatgaaccataacgatttaatctgggtgcatgattaccatttgatgttggttccggaaatgttgagagtcaagattcacgagaagcaactgcaaaacgttaaggtcgggtggttcctgcacacaccattcccttcgagtgaaatttacagaatcttacctgtcagacaagagattttgaagggtgttttgagttgtgatttagtcgggttccacacatacgattatgcaagacatttcttgtcttccgtgcaaagagtgcttaacgtgaacacattgcctaatggggtggaataccagggcagattcgttaacgtaggggccttccctatcggtatcgacgtggacaagttcaccgatgggttgaaaaaggaatccgtacaaaagagaatccaacaattgaaggaaactttcaagggctgcaagatcatagttggtgtcgacaggctggattacatcaaaggtgtgcctcagaagttgcacgccatggaagtgtttctgaacgagcatccagaatggaggggcaaggttgttctggtacaggttgcagtgccaagtcgtggagatgtggaagagtaccaatatttaagatctgtggtcaatgagttggtcggtagaatcaacggtcagttcggtactgtggaattcgtccccatccatttcatgcacaagtctataccatttgaagagctgatttcgttatatgctgtgagcgatgtctgtttggtctcgtccacccgtgatggtatgaacttggtttcctacgaatatattgcttgccaagaagaaaagaaaggttccttaatcctgagtgagttcacaggtgccgcacaatccttgaatggtgctattattgtaaatccttggaacaccgatgatctttctgatgccatcaacgaggccttgactttgcccgatgtaaagaaagaagttaactgggaaaaactttacaaatacatctctaaatacacttctgccttctggggtgaaaatttcgtccatgaattatacagtacatcatcaagctcaacaagctcctctgccaccaaaaactga

SEQ ID NO: 56 - DNA, 2691 bp, Saccharomyces cerevisiae
atgaccaccactgcccaagacaattctccaaagaagagacagcgtatcatcaattgtgtcacgcagctgccctacaaaatccaattgggagaaagcaacgatgactggaaaatatctgctactacaggtaacagcgcattatattcctctctagaataccttcaatttgattctaccgagtacgagcaacacgttgttggttggaccggcgaaataacaagaaccgaacgcaacctgtttactagagaagcgaaagagaaaccacaggatctggacgatgacccactatatttaacaaaagagcagatcaatgggttgactactactctacaagatcatatgaaatctgataaagaggcaaagaccgatactactcaaacagctcccgttaccaataacgttcatcccgtttggctacttagaaaaaaccagagtagatggagaaattacgcggaaaaagtaatttggccaaccttccactacatcttgaatccttcaaatgaaggtgagcaagaaaaaaactggtggtacgactacgtcaagtttaacgaagcttatgcacaaaaaatcggggaagtttacaggaagggtgacatcatctggatccatgactactacctactgctattgcctcaactactgagaatgaaatttaacgacgaatctatcattattggttatttccatcatgccccatggcctagtaatgaatattttcgctgtttgccacgtagaaaacaaatcttagatggtcttgttggggccaatagaatttgtttccaaaatgaatctttctcccgtcattttgtatcgagttgtaaaagattactcgacgcaaccgccaagaaatctaaaaactcttccgatagtgatcaatatcaagtgtctgtgtacggtggtgacgtactcgtagattctttgcctataggtgttaacacaactcaaatactgaaagatgctttcacgaaggatatagattccaaggttctttccatcaagcaagcttatcaaaacaaaaaaattattattggtagagatcgtctggattccgtcagaggcgtcgttcaaaaattaagagcttttgaaactttcttggccatgtatccagaatggcgagatcaagtggtattgatccaggtcagcagtcctactgctaacagaaattccccccaaactatcagattggaacaacaagtcaacgagttggttaattccataaattctgaatatggtaatttgaatttttctcccgtccagcattattatatgagaatccctaaagatgtatacttgtccttactaagagttgcagacttatgtttaatcacaagtgttagagacggtatgaataccactgctttggaatacgtcactgtgaaatctcacatgtcgaactttttatgctacggaaatccattgattttaagtgagttttctggctctagtaacgtattgaaagatgccattgtcgttaacccatgggattcggtggccgtggctaaatctattaacatggctttgaaattggacaaggaagaaaagtccaatttagaatcaaaattatggaaagaagttcctacaattcaagattggactaataagtttttgagttcattaaaggaaaaggcgtcatctgatgatgatgtggaaaggaaaatgactccagcacttaatagacctgttcttttagaaaactacaagcaggctaagcgtagattattcctttttgattacgatggtactttgaccccaattgtcaaagacccagctgcagctattccatcggcaagactttatacaattctacaaaaattatgtgccgatcctcataatcaaatctggattatttctggtcgtgaccagaagtttttgaacaagtggttaggcggtaaacttcctcaactgggtctaagtgcggagcatggatgtttcatgaaagatgtttcttgccaagattgggtcaatttgaccgaaaaagttgatatgtcttggcaagtacgcgtcaatgaagtgatggaagaatttaccacaaggaccccaggttcattcatcgaaagaaagaaagtcgctctaacttggcattatagacgtaccgttccagaattgggtgaattccacgccaaagaactgaaagaaaaattgttatcatttactgatgacttcgatttagaggtcatggatggtaaagcaaacattgaagttcgtccaagattcgtcaacaaaggtgaaatagtcaagagactagtctggcatcaacatggcaaaccacaggacatgttgaagggaatcagtgaaaaactacctaaggatgaaatgcctgattttgtattatgtctgggtgatgacttcactgacgaagacatgtttagacagttgaataccattgaaacttgttggaaagaaaaatatcctgaccaaaaaaatcaatggggcaactacggattctatcctgtcactgtgggatctgcatccaagaaaactgtcgcaaaggctcatttaaccgatcctcagcaagtcctggagactttaggtttacttgttggtgatgtctctctcttccaaagtgctggtacggtcgacctggattccagaggtcatgtcaagaatagtgagagcagtttgaaatcaaagctagcatctaaagcttatgttatgaaaagatcggcttcttacaccggcgcaaaggtttga

SEQ ID NO: 57 - protein, 250 aa, Saccharomyces cerevisiae
MPLTTKPLSLKINAALFDVDGTIIISQPAIAAFWRDFGKDKPYFDAEHVIHISHGWRTYDAIAKFAPDFADEEYVNKLEGEIPEKYGEHSIEVPGAVKLCNALNALPKEKWAVATSGTRDMAKKWFDILKIKRPEYFITANDVKQGKPHPEPYLKGRNGLGFPINEQDPSKSKVVVFEDAPAGIAAGKAAGCKIVGIATTFDLDFLKEKGCDIIVKNHESIRVGEYNAETDEVELIFDDYLYAKDDLLKW

SEQ ID NO: 58 - protein, 250 aa, Saccharomyces cerevisiae
MGLTTKPLSLKVNAALFDVDGTIIISQPAIAAFWRDFGKDKPYFDAEHVIQVSHGWRTFDAIAKFAPDFANEEYVNKLEAEIPVKYGEKSIEVPGAVKLCNALNALPKEKWAVATSGTRDMAQKWFEHLGIRRPKYFITANDVKQGKPHPEPYLKGRNGLGYPINEQDPSKSKVVVFEDAPAGIAAGKAAGCKIIGIATTFDLDFLKEKGCDIIVKNHESIRVGGYNAETDEVEFIFDDYLYAKDDLLKW

SEQ ID NO: 59 - DNA, 2995 bp, Artificial Sequence
  Synthetic polynucleotide
tgagctccgggtgggaggaaggcgcggcaattagaatgtgtgggtgcggaagctcgccgctcccatcaagagagtggaagacgtatggtctgggtgcgaagtaccaccacgtttctttttcatctcttaagtgggattcttacgaaacacgtcacagggtcaaaagaaagagaacaaaagcaatattgtaattgtctcagtccacggcaatgacatggcatggccccgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttctcgaggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgacaacatcaaatacctacaaattctatctaaacggtgaatggagagaatcttcctctggagaaactattgagataccatcaccatacttacatgaagtgatcggacaggttcaagcaatcactagaggagaggttgacgaagcgattgctagcgctaaggaagcacagaaatcttgggctgaggcatctctacaagatagagctaagtacttgtacaaatgggcagatgaattggtaaacatgcaagacgaaatcgccgatatcatcatgaaggaagtgggcaagggttacaaagacgctaaaaaggaggttgttagaaccgccgatttcatcagatacaccattgaagaggcactccatatgcacggtgaatccatgatgggcgattcatttcctggtggaacaaaatctaagctagcaataatccaaagagcgcctctgggtgtagtcttagccatcgctccattcaattaccctgtaaacctttctgctgcaaaattggcaccagccttaattatgggtaacgctgtgatattcaagccagcaactcagggtgctatttccggcatcaaaatggttgaagctttgcataaggctggtttgccaaagggtttggttaacgttgccacaggtagaggtagcgtcataggcgattatttggtcgaacacgaagggataaacatggtttccttcaccggtggcactaacactggtaagcatttagcaaaaaaggcctcaatgattccattagtcttggaacttggtggcaaagatccaggcatcgttcgtgaagatgcagacctacaagatgctgcgaatcatatcgtatctggtgcgttcagttactcagggcagagatgtacagccattaagagagtccttgttcatgaaaatgttgctgatgaactggtatcattggttaaggaacaagtggcaaagctttctgtgggatcaccagagcaagattcaacaattgttcctctgattgacgataagtccgctgattttgttcagggtttagtggacgatgcagtcgaaaagggcgctacaattgtcattgggaacaagagagaacgtaacctaatctacccaacattgattgatcacgtcacagaggaaatgaaagttgcctgggaggaaccattcggtcctattcttccaattattagagttagtagcgacgagcaagctattgaaattgcaaataagagtgagttcggattacaagcttctgtgtttaccaaagacataaacaaggcattcgcaatcgcaaataagattgagactggttcagtgcaaatcaacggtagaacagagagaggaccagatcactttccttttatcggggttaagggatctgggatgggtgcccaaggcatcagaaagtctttggaatctatgactagagaaaaagttactgtcttaaatctcgtatgattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacg

(19) United States
(12) Patent Application Publication
(10) Pub. No.: US 2021/0062230 A1
(43) Pub. Date: Mar. 4, 2021

(54) METHODS FOR ETHANOL PRODUCTION USING ENGINEERED YEAST

(71) Applicant: Cargill, Incorporated, Wayzata, MN (US)

(72) Inventors: Gregory M. Poynter, St. Paul, MN (US); Brian J. Rush, Minneapolis, MN (US); Sneha Srikrishnan, Boston, MA (US); Dawn Thompson, Boston, MA (US); Arthur Shockley, Boston, MA (US); Brynne Kohman, Boston, MA (US); Joshua Dunn, Boston, MA (US)

(73) Assignee: Cargill, Incorporated, Wayzata, MN (US)

(21) Appl. No.: 17/041,258

(22) PCT Filed: Mar. 27, 2019
(86) PCT No.: PCT/US2019/024330

§ 371 Date: Sep. 24, 2020

Related U.S. Application Data
(60) Provisional application No. 62/648,679, filed on Mar. 27, 2018.

(51) Int. Cl. C12P 7/06; C12N 1/18; C12N 9/02; C12N 9/34; C12N 9/10; C12N 9/16; C12P 19/12

(52) U.S. Cl. CPC C12P 7/06; C12N 1/18; C12N 9/0008; C12Y 102/01009; C12P 19/12; C12N 9/1051; C12Y 204/01015; C12N 9/16; C12Y 301/03012; C12N 9/2428

(57) ABSTRACT

Aspects of the disclosure provide engineered microbes for ethanol production. Methods for microbe engineering and culturing are also provided herein. Such engineered microbes exhibit enhanced capabilities for ethanol production.

### RELATED APPLICATIONS

This application claims the benefit under 35 U.S.C. § 119(e) of U.S. Provisional Application Ser. No. 62/648,679, entitled “METHODS FOR ETHANOL PRODUCTION USING ENGINEERED YEAST” filed on Mar. 27, 2018, which is herein incorporated by reference in its entirety.

### FIELD

The disclosure relates to the production of ethanol through genetic engineering.

### BACKGROUND

Ethanol is a renewable biofuel that can be produced through fermentation of natural products. Ethanol produced by fermentation has numerous industrial applications including producing products such as solvents, extractants, antifreeze, and as an intermediate in the synthesis of various organic chemicals. Ethanol is also widely used in industries such as coatings, printing inks, and adhesives. Microorganisms, including yeast, can produce ethanol by fermentation of various substrates, including sugars and starches. Advantages of using yeast for production of ethanol include the ability to use a range of substrates, tolerance to high ethanol concentrations, and the ability to produce large ethanol yields. (Mohd Azhar et al., Biochem Biophys Rep (2017) 10:52-61). However, production of ethanol using yeast fermentation also leads to production of by-products.

### SUMMARY

Aspects of the present disclosure relate to the development of novel engineered yeast and methods of using the novel engineered yeast to produce ethanol. Surprisingly, engineered yeast described herein produce high ethanol yields without exhibiting a fermentation penalty, and produce reduced levels of by-products, such as glycerol.

Aspects of the disclosure relate to engineered yeast comprising: a recombinant nucleic acid encoding a glyceraldehyde-3-phosphate dehydrogenase (E.C. 1.2.1.9); reduced or eliminated expression of a gene encoding a glycerol-3-phosphate phosphatase (E.C. 3.1.3.21); and a recombinant nucleic acid encoding a glucoamylase, wherein the yeast is capable of producing at least 100 g/kg of ethanol and producing less than 1.5 g/kg residual glucose in 48 hours under Test 1 conditions.

In some embodiments, the engineered yeast is a post-whole-genome duplication yeast species. In some embodiments, the yeast is Saccharomyces cerevisiae (S. cerevisiae).

In some embodiments, the engineered yeast produces an ethanol yield that is at least 0.5% higher than a control strain. In some embodiments, the ethanol yield is determined by the following: (Ethanol Titer at Time final−Ethanol Titer at Time zero) divided by Total Glucose Equivalents at Time zero. In some embodiments, the engineered yeast produces 30% less glycerol, 40% less glycerol, or 50% less glycerol than a control strain. In some embodiments, glycerol production is determined by Test 4.

In some embodiments, the glucoamylase (GA) has at least 80%, at least 85%, at least 90%, or at least 95% sequence identity to SEQ ID NO:38 (Saccharomycopsis fibuligera GA). In some embodiments, the GA has at least 80%, at least 85%, at least 90%, or at least 95% sequence identity to SEQ ID NO:39 (Rhizopus oryzae amyA). In some embodiments, the GA has at least 80%, at least 85%, at least 90%, or at least 95% sequence identity to SEQ ID NO:41 (Rhizopus microsporus GA). In some embodiments, the GA has at least 80%, at least 85%, at least 90%, or at least 95% sequence identity to SEQ ID NO:40 (Rhizopus delemar GA).

In some embodiments, the nucleic acid encoding a glyceraldehyde-3-phosphate dehydrogenase (E.C. 1.2.1.9) has at least 80%, at least 85%, at least 90%, or at least 95% sequence identity to SEQ ID NO: 45. In some embodiments, the nucleic acid encoding a glyceraldehyde-3-phosphate dehydrogenase (E.C. 1.2.1.9) encodes a protein that has at least 80%, at least 85%, at least 90%, or at least 95% sequence identity to SEQ ID NO: 42. In some embodiments, the engineered yeast comprises a nucleic acid having at least 80%, at least 85%, at least 90%, or at least 95% sequence identity to SEQ ID NO: 59.

In some embodiments, the engineered yeast has reduced or eliminated expression of a glycerol-3-phosphate dehydrogenase (E.C. 1.1.1.8).

In some embodiments, the engineered yeast is Saccharomyces cerevisiae and the engineered yeast has reduced or eliminated expression of GPP1, GPP2, GPD1, or GPD2. In some embodiments, the engineered yeast is Saccharomyces cerevisiae and the engineered yeast has reduced or eliminated expression of GPP1. In some embodiments, the engineered yeast is Saccharomyces cerevisiae and the engineered yeast has reduced or eliminated expression of GPP2. In some embodiments, the engineered yeast is Saccharomyces cerevisiae and the engineered yeast has reduced or eliminated expression of GPD1. In some embodiments, the engineered yeast is Saccharomyces cerevisiae and the engineered yeast has reduced or eliminated expression of GPD2.

In some embodiments, the engineered yeast further comprises a nucleic acid encoding a trehalose-6-phosphate synthase (Tps1; E.C. 2.4.1.15). In some embodiments, the nucleic acid encoding a trehalose-6-phosphate synthase (Tps1; E.C. 2.4.1.15) has at least 80%, at least 85%, at least 90%, or at least 95% sequence identity to SEQ ID NO: 55. In some embodiments, nucleic acid encoding a trehalose-6-phosphate synthase (Tps1; E.C. 2.4.1.15) encodes a protein that has at least 80%, at least 85%, at least 90%, or at least 95% sequence identity to SEQ ID NO: 43.

In some embodiments, the engineered yeast further comprises a nucleic acid encoding a trehalose-6-phosphate synthase (Tps2; EC 3.1.3.12). In some embodiments, the nucleic acid encoding a trehalose-6-phosphate synthase (Tps2; EC 3.1.3.12) has at least 80%, at least 85%, at least 90%, or at least 95% sequence identity to SEQ ID NO: 56. In some embodiments, the nucleic acid encoding a trehalose-6-phosphate synthase (Tps2; EC 3.1.3.12) encodes a protein that has at least 80%, at least 85%, at least 90%, or at least 95% sequence identity to SEQ ID NO: 44.

Aspects of the disclosure relate to engineered S. cerevisiae yeast comprising: a recombinant nucleic acid encoding a glyceraldehyde-3-phosphate dehydrogenase (E.C. 1.2.1.9); and reduced or eliminated expression of a gene encoding a glycerol-3-phosphate phosphatase (E.C. 3.1.3.21), wherein the yeast is capable of producing at least 100 g/kg of ethanol and producing less than 1.5 g/kg residual glucose in 48 hours under Test 2 conditions.

In some embodiments, the engineered S. cerevisiae yeast produces an ethanol yield that is at least 0.5% higher than a control strain. In some embodiments, the ethanol yield is determined by the following formula: (Ethanol Titer at Time final−Ethanol Titer at Time zero) divided by Total Glucose Equivalents at Time zero. In some embodiments, the engineered yeast produces 30% less glycerol, 40% less glycerol, or 50% less glycerol than a control strain. In some embodiments, glycerol production is determined by Test 4.

In some embodiments, the GA has at least 80%, at least 85%, at least 90%, or at least 95% sequence identity to SEQ ID NO:38 (Saccharomycopsis fibuligera GA). In some embodiments, the GA has at least 80%, at least 85%, at least 90%, or at least 95% sequence identity to SEQ ID NO:39 (Rhizopus oryzae amyA). In some embodiments, the GA has at least 80%, at least 85%, at least 90%, or at least 95% sequence identity to SEQ ID NO:41 (Rhizopus microsporus GA). In some embodiments, the GA has at least 80%, at least 85%, at least 90%, or at least 95% sequence identity to SEQ ID NO:40 (Rhizopus delemar GA).

Aspects of the disclosure relate to engineered yeast comprising an exogenous nucleic acid encoding a glyceraldehyde-3-phosphate dehydrogenase (E.C. 1.2.1.9), and an exogenous nucleic acid encoding a GA having 80% or greater identity to SEQ ID NO:38 (Saccharomycopsis fibuligera GA), SEQ ID NO:41 (Rhizopus microsporus GA), SEQ ID NO:40 (Rhizopus delemar GA), or SEQ ID NO:39 (Rhizopus oryzae amyA) wherein the yeast is capable of producing at least 100 g/kg of ethanol and having less than 1.5 g/kg residual glucose in 48 hours under Test 1 conditions.

In some embodiments, the yeast is a post-whole-genome duplication yeast species. In some embodiments, the yeast is S. cerevisiae.

In some embodiments, the engineered yeast produces an ethanol yield that is at least 0.5% higher than a control strain. In some embodiments, the ethanol yield is determined by the following formula: (Ethanol Titer at Time final−Ethanol Titer at Time zero) divided by Total Glucose Equivalents at Time zero.

In some embodiments, the engineered yeast produces 30% less glycerol, 40% less glycerol, or 50% less glycerol than a control strain. In some embodiments, glycerol production is determined by Test 4.

In some embodiments, the engineered yeast has reduced or eliminated expression of a gene encoding a glycerol-3-phosphate phosphatase (E.C. 3.1.3.21).

In some embodiments, the nucleic acid encoding a glyceraldehyde-3-phosphate dehydrogenase (E.C. 1.2.1.9) has at least 80%, at least 85%, at least 90%, or at least 95% sequence identity to SEQ ID NO: 45. In some embodiments, the nucleic acid encoding a glyceraldehyde-3-phosphate dehydrogenase (E.C. 1.2.1.9) encodes a protein that has at least 80%, at least 85%, at least 90%, or at least 95% sequence identity to SEQ ID NO: 42. In some embodiments, the engineered yeast comprises a nucleic acid having at least 80%, at least 85%, at least 90%, or at least 95% sequence identity to SEQ ID NO: 59.

In some embodiments, the engineered yeast has reduced or eliminated expression of a glycerol-3-phosphate dehydrogenase (E.C. 1.1.1.8).

In some embodiments, the engineered yeast is Saccharomyces cerevisiae and the engineered yeast has reduced or eliminated expression of GPP1, GPP2, GPD1, or GPD2. In some embodiments, the engineered yeast is Saccharomyces cerevisiae and the engineered yeast has reduced or eliminated expression of GPP1. In some embodiments, wherein the engineered yeast is Saccharomyces cerevisiae and the engineered yeast has reduced or eliminated expression of GPP2. In some embodiments, the engineered yeast is Saccharomyces cerevisiae and the engineered yeast has reduced or eliminated expression of GPD1. In some embodiments, the engineered yeast is Saccharomyces cerevisiae and the engineered yeast has reduced or eliminated expression of GPD2.

In some embodiments, the engineered yeast further comprises a nucleic acid encoding a trehalose-6-phosphate synthase (Tps1; E.C. 2.4.1.15). In some embodiments, the nucleic acid encoding a trehalose-6-phosphate synthase (Tps1; E.C. 2.4.1.15) has at least 80%, at least 85%, at least 90%, or at least 95% sequence identity to SEQ ID NO: 55. In some embodiments, nucleic acid encoding a trehalose-6-phosphate synthase (Tps1; E.C. 2.4.1.15) encodes a protein that has at least 80%, at least 85%, at least 90%, or at least 95% sequence identity to SEQ ID NO: 43.

In some embodiments, the engineered yeast further comprises a nucleic acid encoding a trehalose-6-phosphate synthase (Tps2; EC 3.1.3.12). In some embodiments, the nucleic acid encoding a trehalose-6-phosphate synthase (Tps2; EC 3.1.3.12) has at least 80%, at least 85%, at least 90%, or at least 95% sequence identity to SEQ ID NO: 56. In some embodiments, the nucleic acid encoding a trehalose-6-phosphate synthase (Tps2; EC 3.1.3.12) encodes a protein that has at least 80%, at least 85%, at least 90%, or at least 95% sequence identity to SEQ ID NO: 44.

Aspects of the disclosure relate to methods for producing ethanol comprising fermenting engineered yeast described herein with a fermentation substrate. In some embodiments, the fermentation substrate comprises starch. In some embodiments, the fermentation substrate comprises glucose. In some embodiments, the fermentation substrate comprises sucrose. In some embodiments, the starch is obtained from corn, wheat and/or cassava. In some embodiments, the method includes supplementation with glucoamylase.

Aspects of the present disclosure relate to methods for producing trehalose comprising fermenting any of the engineered yeast disclosed herein with a fermentation substrate.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. For purposes of clarity, not every component may be labeled in every drawing. In the drawings:

FIG. 1 is a graph showing ethanol production in corn mash with Strain 1-22, which contains the Bacillus cereus (Bc) gapN gene at the GPP1 locus in a Rhizopus oryzae (Ro) glucoamylase strain background.

FIG. 2 is a table showing ethanol yield in corn mash with Strain 1-22.

FIGS. 3A-C. FIG. 3A is a graph showing titers of ethanol with Strain 1-22. FIG. 3B is a graph showing titers of residual glucose with Strain 1-22. FIG. 3C is a graph showing titers of glycerol with Strain 1-22.

FIG. 4 is a graph showing a comparison of ethanol production with Strains 1-20 and 1-22.

FIG. 5 is a table showing production of ethanol with Strain 1-22 in Light Steep Water/Liquifact (corn wet mill feedstock) airlock shake flasks.

FIG. 6 is a graph showing ethanol titers in corn mash.

FIG. 7 is a graph showing residual glucose in corn mash.

FIG. 8 is a graph showing glycerol titers in corn mash.

FIG. 9 is a graph showing the ethanol titer increase of Strain 1-25 relative to Strain 1 in corn mash at 47 hrs.

FIG. 10A-B. FIG. 10A is a graph showing the glycerol reduction of Strain 1-25 relative to Strain 1 in corn mash. FIG. 10B is a graph showing residual glucose at the end of fermentation (47 hrs) in corn mash.

FIG. 11 is a graph showing glycerol titer at 48 hrs with the indicated strains.

FIG. 12 is a graph showing ethanol titer at 48 hrs with the indicated strains.

FIG. 13 is a graph showing residual glucose at 48 hrs with the indicated strains.

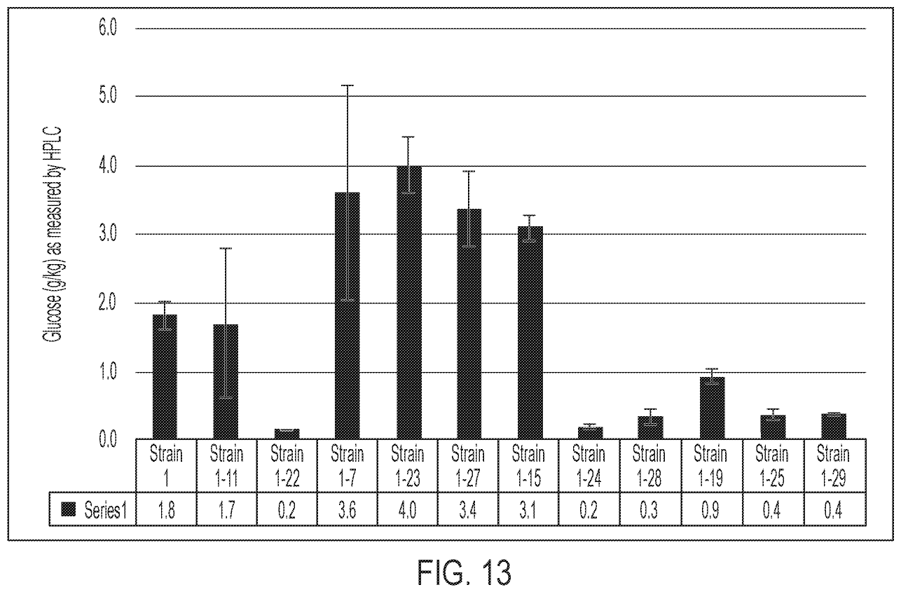

### DETAILED DESCRIPTION

Aspects of the disclosure relate to genetically engineered microorganisms for production of ethanol. Previously reported attempts to engineer yeast to reduce production of by-products in ethanol fermentation were hampered by fermentation penalties. Surprisingly, engineered yeast described herein exhibit increased ethanol titers without a fermentation penalty, and produce reduced amounts of by-products, including glycerol. Accordingly, novel engineered yeast described herein represent an unexpectedly efficient new approach for producing ethanol through fermentation.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations of thereof herein, is meant to encompass the items listed thereafter and equivalents thereof as well as additional items.

### Reduced Glycerol Production

### Glycerol-3-Phosphate Phosphatase

Engineered yeast strains described herein can include genetic modifications in one or more enzymes involved in glycerol production. For example, engineered yeast strains described herein can have reduced or eliminated expression of one or more genes encoding a glycerol-3-phosphate phosphatase (Gpp; corresponding to E.C. 3.1.3.21; also known as “glycerol-1-phosphatase”). Glycerol-3-phosphate phosphatase enzymes hydrolyze glycerol-3-phosphate into glycerol, and thereby regulate the cellular levels of glycerol-3-phosphate, a metabolic intermediate of glucose, lipid and energy metabolism (Mugabo et al., PNAS (2016) 113:E430-439).

Saccharomyces cerevisiae (S. cerevisiae) has two glycerol-3-phosphate phosphatase paralogs, referred to as Gpp1p and Gpp2p, encoded by the GPP1 (UniProt No. P41277) and GPP2 (UniProt No. P40106) genes, respectively (Norbeck et al. (1996) J. Biol. Chem. 271(23):13875-81; Pahlman et al. (2001) J. Biol. Chem. 276(5):3555-63). In some embodiments, engineered yeast described herein, such as S. cerevisiae, has reduced or eliminated expression of GPP1. In other embodiments, engineered yeast described herein, such as S. cerevisiae, has reduced or eliminated expression of GPP2. In other embodiments, engineered yeast described herein, such as S. cerevisiae, has reduced or eliminated expression of both GPP1 and GPP2.

The amino acid sequence of Gpp1p (UniProt No. P41277) (SEO ID NO: 57) is:

MPLTTKPLSLKINAALFDVDGTIIISQPAIAAFWRDFGKDKPYFDAEHVIH
ISHGWRTYDAIAKFAPDFADEEYVNKLEGEIPEKYGEHSIEVPGAVKLCNA
LNALPKEKWAVATSGTRDMAKKWFDILKIKRPEYFITANDVKQGKPHPEPY
LKGRNGLGFPINEQDPSKSKVVVFEDAPAGIAAGKAAGCKIVGIATTFDLD
FLKEKGCDIIVKNHESIRVGEYNAETDEVELIFDDYLYAKDDLLKW.

The amino acid sequence of Gpp2p (UniProt No. P40106) (SEQ ID NO: 58) is:

MGLTTKPLSLKVNAALFDVDGTIIISQPAIAAFWRDFGKDKPYFDAEHVIQ
VSHGWRTFDAIAKFAPDFANEEYVNKLEAEIPVKYGEKSIEVPGAVKLCNA
LNALPKEKWAVATSGTRDMAQKWFEHLGIRRPKYFITANDVKQGKPHPEPY
LKGRNGLGYPINEQDPSKSKVVVFEDAPAGIAAGKAAGCKIIGIATTFDLD
FLKEKGCDIIVKNHESIRVGGYNAETDEVEFIFDDYLYAKDDLLKW.

It should be appreciated that any means of achieving reduced or eliminated expression of a gene encoding a glycerol-3-phosphate phosphatase enzyme is compatible with aspects of the invention. For example, reduced or eliminated expression of a gene encoding a glycerol-3-phosphate phosphatase can be achieved by disrupting the sequence of the gene and/or one or more regulatory regions controlling expression of the gene, such as by introducing one or more mutations or insertions into the sequence of the gene or into one or more regulatory regions controlling expression of the gene.

In some embodiments, expression of a gene encoding a glycerol-3-phosphate phosphatase enzyme, such as the GPP1 gene, is reduced by at least approximately 10%, 20%, 30%, 40%, 50%, 60%, 70%, 80%, 90% or 100%. In some embodiments, expression of the gene encoding a glycerol-3-phosphate phosphatase enzyme, such as the GPP1 gene is eliminated. Expression of a gene encoding a glycerol-3-phosphate phosphatase enzyme, such as a GPP1 gene, can be eliminated by any means known to one of ordinary skill in the art, such as by insertion of a nucleic acid fragment into the GPP1 locus or regulatory regions surrounding the GPP1 locus.

In some embodiments, engineered yeast described herein, such as S. cerevisiae, is diploid and has reduced or eliminated expression of both copies of the GPP1 gene. In some embodiments, engineered yeast described herein, such as S. cerevisiae, is diploid and contains a deletion and/or insertion in both copies of the GPP1 gene.

### Glycerol-3-Phosphate Dehydrogenase (E.C. 1.1.1.8)

Engineered yeast described herein can have reduced or eliminated expression of one or more genes encoding a glycerol-3-phosphate dehydrogenase (Gpd; corresponding to E.C. 1.1.1.8).

S. cerevisiae has two glycerol-3-phosphate dehydrogenases, referred to as Gpd1p and Gpd2p, encoded by the GPD1 (UniProt No. Q00055) and GPD2 (UniProt No. P41911) genes, respectively. In some embodiments, engineered yeast described herein, such as S. cerevisiae, has reduced or eliminated expression of GPD1. In other embodiments, engineered yeast described herein, such as S. cerevisiae, has reduced or eliminated expression of GPD2. In other embodiments, engineered yeast described herein, such as S. cerevisiae, has reduced or eliminated expression of both GPD1 and GPD2.

It should be appreciated that any means of achieving reduced or eliminated expression of a gene encoding a glycerol-3-phosphate dehydrogenase enzyme is compatible with aspects of the invention. For example, reduced or eliminated expression of a gene encoding a glycerol-3-phosphate dehydrogenase can be achieved by disrupting the sequence of the gene and/or one or more regulatory regions controlling expression of the gene, such as by introducing one or more mutations or insertions into the sequence of the gene or into one or more regulatory regions controlling expression of the gene.

In some embodiments, expression of a gene encoding a glycerol-3-phosphate dehydrogenase enzyme, such as the GPD1 gene, is reduced by at least approximately 10%, 20%, 30%, 40%, 50%, 60%, 70%, 80%, 90% or 100%. In some embodiments, expression of the gene encoding a glycerol-3-phosphate dehydrogenase enzyme, such as the GPD1 gene is eliminated. Expression of a gene encoding a glycerol-3-phosphate dehydrogenase enzyme, such as a GPD1 gene, can be eliminated by any means known to one of ordinary skill in the art, such as by insertion of a nucleic acid fragment into the GPD1 locus or regulatory regions surrounding the GPD1 locus.

In some embodiments, engineered yeast described herein, such as S. cerevisiae, is diploid and has reduced or eliminated expression of both copies of the GPD1 gene. In some embodiments, engineered yeast described herein, such as S. cerevisiae, is diploid and contains a deletion and/or insertion in both copies of the GPD1 gene. In other embodiments, engineered yeast described herein, such as S. cerevisiae, has reduced or eliminated expression of one copy of the GPD1 gene.

In some embodiments, engineered yeast described herein, such as S. cerevisiae, has reduced or eliminated expression of GPP1 and/or GPP2, and also has reduced or eliminated expression of GPD1 and/or GPD2. In certain embodiments, engineered yeast described herein, such as S. cerevisiae, has reduced or eliminated expression of two copies of GPP1 and also has reduced or eliminated expression of one copy of GPD1.

### Glyceraldehyde-3-Phosphate Dehydrogenase (GAPN; E.C. 1.2.1.9)

Engineered yeast described herein recombinantly express one or more nucleic acids encoding a glyceraldehyde-3-phosphate dehydrogenase enzyme (gapN; corresponding to E.C. 1.2.1.9; also known as “NADP-dependent non-phosphorylating glyceraldehyde-3-phosphate dehydrogenase”). GapN enzymes convert D-glyceraldehyde 3-phosphate to 3-phospho-D-glycerate (Rosenberg et al., J Biol Chem (1955) 217:361-71).

It should be appreciated that the recombinant nucleic acid encoding a gapN enzyme can come from any source. An engineered yeast that recombinantly expresses a nucleic acid encoding a gapN enzyme may or may not contain an endogenous gene encoding a gapN enzyme. In some embodiments, the engineered yeast that recombinantly expresses a nucleic acid encoding a gapN enzyme does not contain an endogenous copy of a gene encoding a gapN enzyme. Accordingly, in such embodiments, the nucleic encoding a gapN enzyme is derived from a species or organism different from the engineered yeast.

In other embodiments, the engineered yeast that recombinantly expresses a nucleic acid encoding a gapN enzyme does contain an endogenous copy of a gene encoding a gapN enzyme. In some such embodiments, the endogenous copy of the gene encoding a gapN enzyme, or a regulatory region for the gene, such as a promoter, is engineered to increase expression of the gene encoding a gapN enzyme. In other such embodiments, a nucleic acid encoding a gapN enzyme is introduced into the yeast. In such embodiments, the nucleic acid encoding the gapN enzyme that is introduced into the yeast may be derived from the same species or organism as the engineered yeast in which it is expressed, or may be derived from a different species or organism than the engineered yeast in which it is expressed.

In some embodiments, the recombinant nucleic acid encoding a gapN enzyme comprises a Bacillus cereus gene (e.g., GAPN, corresponding to UniProt No. Q2HQS1). In some embodiments, the recombinant nucleic acid encoding a GapN enzyme, or a portion thereof, is codon-optimized. In some embodiments, the recombinant nucleic acid encoding a gapN enzyme, or a portion thereof, comprises SEQ ID NO: 45.

In some embodiments, the recombinant nucleic acid encoding a gapN enzyme, or portion thereof, has at least or about 50%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, at least or about 99.5%, or at least or about 99.9% sequence identity to the sequence of SEQ ID NO:45.

In some embodiments the gapN protein comprises SEQ ID NO:42. In some embodiments the gapN protein has at least or about 50%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, at least or about 99.5%, or at least or about 99.9% sequence identity to the sequence of SEQ ID NO:42.

One of ordinary skill in the art would understand that a GAPN gene could be derived from any source and could be engineered using routine methods, such as to improve expression in a host cell.

### Trehalose Biosynthesis

Engineered yeast described herein can recombinantly express one or more genes encoding one or more proteins involved in trehalose biosynthesis (Gancedo et al. (2004) FEMS Yeast Research 4:351-359). Non-limiting examples of enzymes involved in trehalose biosynthesis include trehalose-6-phosphate synthase (Tps1; E.C. 2.4.1.15) and trehalose-6-phosphate phosphatase (Tps2; EC 3.1.3.12).

In S. cerevisiae, Tps1 is encoded by the TPS1 gene (UniProt No. C7GY09), and Tps2 is encoded by the TPS2 gene (UniProt No. P31688). It should be appreciated that the recombinant nucleic acid encoding a Tps1 or Tps2 enzyme can come from any source. An engineered yeast cell that recombinantly expresses a nucleic acid encoding a Tps1 or Tps2 enzyme may or may not contain an endogenous gene encoding a Tps1 or Tps2 enzyme. In some embodiments, the engineered yeast cell that recombinantly expresses a nucleic acid encoding a Tps1 or Tps2 enzyme does not contain an endogenous copy of a gene encoding a Tps1 or Tps2 enzyme. Accordingly, in such embodiments, the nucleic encoding a Tps1 or Tps2 enzyme is derived from a species or organism different from the engineered yeast cell.

In other embodiments, the engineered yeast that recombinantly expresses a nucleic acid encoding a Tps1 or Tps2 enzyme does contain an endogenous copy of a gene encoding a Tps1 or Tps2 enzyme. In some such embodiments, the endogenous copy of the gene encoding a Tps1 or Tps2 enzyme, or a regulatory region for the gene, such as a promoter, is engineered to increase expression of the gene encoding a Tps1 or Tps2 enzyme. In other embodiments, a nucleic acid encoding a Tps1 or Tps2 enzyme is introduced into the yeast. In such embodiments, the nucleic acid encoding the Tps1 or Tps2 enzyme that is introduced into the yeast may be derived from the same species or organism as the engineered yeast in which it is expressed, or may be derived from a different species or organism than the engineered yeast in which it is expressed.

In some embodiments, the recombinant nucleic acid encoding a Tps1 or Tps2 enzyme comprises an S. cerevisiae gene (e.g., corresponding to UniProt Nos. C7GY09 or P31688). In some embodiments, Tps1 corresponds to SEQ ID NO: 43. In some embodiments, Tps2 corresponds to SEQ ID NO: 44. One of ordinary skill in the art would understand that a TPS1 or TPS2 gene could be derived from any source and could be engineered using routine methods, such as to improve expression in a host cell.

### Glucoamylases

Engineered yeast described herein recombinantly express a nucleic acid encoding a glucoamylase enzyme (E.C. 3.2.1.3). Glucoamylase enzymes hydrolyze terminal 1,4-linked alpha-D-glucose residues successively from non-reducing ends of amylose chains to release free glucose (see e.g., Mertens et al., Curr Microbiol (2007) 54:462-6).

It should be appreciated that the nucleic acid encoding a glucoamylase enzyme can come from any source. An engineered yeast that recombinantly expresses a nucleic acid encoding a glucoamylase enzyme may or may not contain an endogenous gene encoding a glucoamylase enzyme. In some embodiments, the engineered yeast that recombinantly expresses a nucleic acid encoding a glucoamylase enzyme does not contain an endogenous copy of a gene encoding a glucoamylase enzyme. Accordingly, in such embodiments, the nucleic encoding a glucoamylase enzyme is derived from a species or organism different from the engineered yeast.

In other embodiments, the engineered yeast that recombinantly expresses a nucleic acid encoding a glucoamylase enzyme does contain an endogenous copy of a gene encoding a glucoamylase enzyme. In some such embodiments, the endogenous copy of the gene encoding a glucoamylase enzyme, or a regulatory region for the gene, such as a promoter, is engineered to increase expression of the gene encoding a glucoamylase enzyme. In other embodiments, a nucleic acid encoding a glucoamylase enzyme is introduced into the yeast. In such embodiments, the nucleic acid encoding the glucoamylase enzyme that is introduced into the yeast may be derived from the same species or organism as the engineered yeast in which it is expressed, or may be derived from a different species or organism than the engineered yeast in which it is expressed.

In some embodiments, the recombinant nucleic acid encoding a glucoamylase enzyme comprises a Saccharomycopsis fibuligera gene (e.g., corresponding to UniProt No. Q8TFE5). In some embodiments, the recombinant nucleic acid encoding a glucoamylase enzyme, or a portion thereof, is codon-optimized. In some embodiments, the recombinant nucleic acid encoding a glucoamylase enzyme, or a portion thereof, comprises SEQ ID NO: 46 through 49.

In some embodiments, the recombinant nucleic acid encoding a glucoamylase has at least or about 50%, at least or about 60%, at least or about 70%, at least or about 80%, at least or about 85%, at least or about 90%, at least or about 95%, at least or about 96%, at least or about 97%, at least or about 98%, at least or about 99%, at least or about 99.5%, at least or about 99.9%, or at least or about 100% sequence identity to the nucleic acid sequence of SEQ ID NO: 46 through 49.

In some embodiments, the glucoamylase has at least or about 50%, at least or about 60%, at least or about 70%, at least or about 80%, at least or about 85%, at least or about 90%, at least or about 95%, at least or about 96%, at least or about 97%, at least or about 98%, at least or about 99%, at least or about 99.5%, at least or about 99.9%, or at least or about 100% sequence identity to the protein sequence of SEQ ID NO: 38.

In some embodiments, the recombinant nucleic acid encoding a glucoamylase enzyme comprises a Rhizopus delemar gene (e.g., RO3G_00082, corresponding to UniProt No. I1BGP8). In some embodiments, the recombinant nucleic acid encoding a glucoamylase enzyme, or a portion thereof, is codon-optimized. In some embodiments, the recombinant nucleic acid encoding a glucoamylase enzyme, or a portion thereof, comprises SEQ ID NO: 52 or 53.

In some embodiments, the recombinant nucleic acid encoding a glucoamylase has at least or about 50%, at least or about 60%, at least or about 70%, at least or about 80%, at least or about 85%, at least or about 90%, at least or about 95%, at least or about 96%, at least or about 97%, at least or about 98%, at least or about 99%, at least or about 99.5%, at least or about 99.9%, or 100% sequence identity to the nucleic acid sequence of SEQ ID NO: 52 or 53.

In some embodiments, the glucoamylase has at least or about 50%, at least or about 60%, at least or about 70%, at least or about 80%, at least or about 85%, at least or about 90%, at least or about 95%, at least or about 96%, at least or about 97%, at least or about 98%, at least or about 99%, at least or about 99.5%, or 100% sequence identity to the protein sequence of SEQ ID NO: 40.

In some embodiments, the recombinant nucleic acid encoding a glucoamylase enzyme comprises a Rhizopus microsporus gene (e.g., corresponding to UniProt No. A0A0C7BD37). In some embodiments, the recombinant nucleic acid encoding a glucoamylase enzyme, or a portion thereof, is codon-optimized. In some embodiments, the recombinant nucleic acid encoding a glucoamylase enzyme, or a portion thereof, comprises SEQ ID NO: 54.

In some embodiments, the recombinant nucleic acid encoding a glucoamylase has at least or about 50%, at least or about 60%, at least or about 70%, at least or about 80%, at least or about 85%, at least or about 90%, at least or about 95%, at least or about 96%, at least or about 97%, at least or about 98%, at least or about 99%, at least or about 99.5%, at least or about 99.9%, or 100% sequence identity to the nucleic acid sequence of SEQ ID NO: 54.

In some embodiments, the glucoamylase comprises at least or about 50%, at least or about 60%, at least or about 70%, at least or about 80%, at least or about 85%, at least or about 90%, at least or about 95%, at least or about 96%, at least or about 97%, at least or about 98%, at least or about 99%, at least or about 99.5%, or 100% sequence identity to the protein sequence of SEQ ID NO: 41.

In some embodiments, the recombinant nucleic acid encoding a glucoamylase enzyme comprises a Rhizopus oryzae gene (e.g., amyA, corresponding to UniProt No. B7XC04). In some embodiments, the recombinant nucleic acid encoding a glucoamylase enzyme, or a portion thereof, is codon-optimized. In some embodiments, the recombinant nucleic acid encoding a glucoamylase enzyme, or a portion thereof, comprises SEQ ID NO: 50 or 51.

In some embodiments, the recombinant nucleic acid encoding a glucoamylase has at least or about 50%, at least or about 60%, at least or about 70%, at least or about 80%, at least or about 85%, at least or about 90%, at least or about 95%, at least or about 96%, at least or about 97%, at least or about 98%, at least or about 99%, at least or about 99.5%, at least or about 99.9%, or 100% sequence identity to the nucleic acid sequence of SEQ ID NO: 50 or 51.

In some embodiments, the glucoamylase has at least or about 50%, at least or about 60%, at least or about 70%, at least or about 80%, at least or about 85%, at least or about 90%, at least or about 95%, at least or about 96%, at least or about 97%, at least or about 98%, at least or about 99%, at least or about 99.5%, or 100% sequence identity to the protein sequence of SEQ ID NO: 39.

### Host Cells

Any type of cell that can be used for fermentation to produce ethanol can be compatible with aspects of the invention, including fungal cells, such as yeast cells. Non-limiting examples of yeast cells include yeast cells obtained from, e.g., Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp. and industrial polyploid yeast strains. In certain embodiments, the yeast cell is a S. cerevisiae cell. Other examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In some embodiments, the cell is from a post-whole-genome duplication yeast species, such as S. cerevisiae (Wolfe (2015) PLoS Biol 13(8): e1002221).

### Fermentation Conditions

Novel methods for the production of ethanol comprising fermenting engineered yeast are provided herein. In some embodiments, a method for producing ethanol includes culturing a cell, such as an engineered cell described herein, with a fermentation substrate, under conditions that result in the production of ethanol.

The fermentation substrate can comprise a starch. Starch can be obtained from a natural source, such as a plant source. Starch can also be obtained from a feedstock with high starch or sugar content, including, but not limited to corn, sweet sorghum, fruits, sweet potato, rice, barley, sugar cane, sugar beets, wheat, cassava, potato, tapioca, arrowroot, peas, or sago. In some embodiments, the fermentation substrate is from lignocellulosic biomass such as wood, straw, grasses or algal biomass, such as microalgae and macroalgae. In some embodiments, the fermentation substrate is from grasses, trees, or agricultural and forestry residues, such as corn cobs and stalks, rice straw, sawdust, and wood chips. A fermentation substrate can also comprise a sugar, such as glucose or sucrose.

In some embodiments, the fermentation substrate comprises a dry grind ethanol feedstock, such as corn mash. In some embodiments, the fermentation substrate comprises a liquefied corn mash (LCM). In some embodiments, the fermentation substrate comprises a corn wet mill feedstock, such as Light Steep Water/Liquifact (LSW/LQ).

Media for fermentation of engineered yeast described herein can be supplemented with various components. For example, media for fermentation of engineered yeast described herein can be supplemented with glucoamylase. In some embodiments, the glucoamylase is Spirizyme™ (Novozymes, Bagsvaerd, Denmark).

In some embodiments, the concentration and amount of a supplemental component, such as glucoamylase, is optimized. For example, in some embodiments, glucoamylase is added at a concentration of about 1%, 5%, 10%, 11%, 12%, 13%, 14%, 15%, 16%, 17%, 18%, 19%, 20%, 21%, 22%, 23%, 24%, 25%, 26%, 27%, 28%, 29%, 30% or more than 30%. In some embodiments, a quantity of glucoamylase is added to achieve a dose of approximately 0.33 AGU/g of Dry Solids. In some embodiments, a quantity of glucoamylase is added to achieve a dose of approximately 0.0825 AGU/g of Dry Solids. In some embodiments, a quantity of glucoamylase is added to achieve a dose of approximately 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1.0 AGU/g of Dry Solids.

It should be appreciated that engineered yeast described herein can be cultured in media of any type and any composition, and the fermentation conditions can be optimized through routine experimentation as would be understood by one of ordinary skill in the art. In some embodiments, the fermentation conditions are optimized for the production of ethanol. Parameters that can be optimized include, but are not limited to, temperature, sugar concentration, pH, fermentation time, agitation rate, and/or inoculum size.

In some embodiments, the temperature of culture medium for an engineered yeast described herein is controlled for optimal ethanol production. (See e.g., Zabed et al., Sci World J (2014):1-11; Charoenchai et al., Am J Enol Vitic (1998) 49:283-8; MarelneCot et al., FEMS Yeast Res (2007) 7:22-32; Liu et al., Bioresour Technol (2008) 99:847-54; Phisalaphong et al., J Biochem Eng (2006) 28:36-43). Multiple factors can influence the optimal temperature for culturing an engineered yeast for the production of ethanol (e.g., cell type, growth media and growth conditions). In some embodiments, the temperature of the culture is between 25 and 40° C., inclusive. In certain embodiments, the temperature is about 25, 26, 27, 28, 29, 30, 31, 32, 33, 34, 35, 36, 37, 38, 39, 40° C., or any value in between. In some embodiments, the temperature is between 30 and 35° C., inclusive or any value in between. In some embodiments, the temperature is approximately 33° C. In certain embodiments, the temperature is approximately 33.3° C.

In some embodiments, the pH of a culture medium described herein is controlled for optimal ethanol production (Lin et al., Biomass-Bioenergy (2012) 47:395-401). In some embodiments, the pH of the culture or a fermentation mixture of an engineered cell described herein is at a range of between 4.0 and 6.0. In some embodiments, the pH is maintained for at least part of the incubation at 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0. In some embodiments, the pH is maintained at a range between 5.0 and 5.5.

In some embodiments, the culture time is controlled for optimal ethanol production (Lin et al., Biomass-Bioenergy (2012) 47:395-401). In some embodiments, an engineered yeast is cultured for approximately 24-72 hours. In some embodiments, an engineered yeast is cultured for approximately 12, 18, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 78, 80, 90, 96 hours, or more than 96 hours. In some embodiments, an engineered yeast described herein is cultured for approximately 48 to 72 hours. In some embodiments, a culture (fermentation) time of about 48 hours is a representative time for commercial-scale ethanol fermentation processes. Accordingly, a 48 hour time point can be used to compare the fermentation performance of different yeast strains.

Reaction parameters can be measured or adjusted during the production of ethanol. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, ethanol concentration, fermentation substrate concentration, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described herein are well known to one of ordinary skill in the art.

Sugar and oligocarbohydrates contents are determined using HPLC with Aminex HPX-87H column (300 mm×7.8 mm) at 60 C, 0.01N sulfuric acid mobile phase, 0.6 mL/min flow rate.

### Assay and Test Conditions

### Test 1

Aspects of the disclosure relate to engineered yeast that is capable of producing at least 100 g/kg of ethanol and producing less than 1.5 g/kg residual glucose in 48 hours under Test 1 conditions, which involve characterization of strains in 33% DS corn mash at 33.3° C.

As used herein “Test 1” conditions refers to the following: Strains are struck to a YPD plate and incubated at 30° C. until single colonies are visible (1-2 days). Cells from a YPD plate are scraped into pH 7.0 sterile phosphate buffer and the optical density (OD600) is measured. Optical density is measured at wavelength of 600 nm with a 1 cm path length using a model Genesys 20 Visible Spectrophotometer (Thermo Scientific). A shake flask is inoculated with the volume of the cell slurry necessary to reach an initial OD600 of 0.1. The inoculation volume is typically around 66 μl. Immediately prior to inoculating, the following materials are added to each 250 ml baffled shake flask: 50 grams of liquified corn mash, 1900 of 500 g/L filter-sterilized urea, and 2.50 of a 100 mg/ml filter sterilized stock of ampicillin. For the shake flasks containing the Ethanol Red® control strain, a quantity of glucoamylase (Spirizyme Fuel HS™ Novozymes; lot NAPM3771) to achieve a dose of 0.33 AGU/g of Dry Solids is added to the flasks, and 0.0825 AGU/g of Dry Solids (or a 25% of the dose provided to Ethanol Red®) of glucoamylase (Spirizyme Fuel HS™ Novozymes; lot NAPM3771) is added to the flasks containing the glucoamylase expressing yeast. Glucoamylase activity is measured using the Glucoamylase Activity Assay (described in the Examples section). Duplicate flasks for each strain are incubated at 33.3° C. with shaking in an orbital shaker at 100 rpm for approximately 48 hours. At 48 hours, 1 ml samples are taken and analyzed for ethanol and glucose concentrations in the broth by high performance liquid chromatography with a refractive index detector.

### Test 2

Aspects of the disclosure relate to engineered yeast, such as S. cerevisiae, that is capable of producing at least 100 g/kg of ethanol and producing less than 1.5 g/kg residual glucose in 48 hours under Test 2 conditions, involving characterizing strains in 33% DS corn mash at 33.3° C.

As used herein “Test 2” conditions refers to the following: Strains are struck to a YPD plate and incubated at 30° C. until single colonies are visible (1-2 days). Cells from a YPD plate are scraped into pH 7.0 sterile phosphate buffer and the optical density (OD600) is measured. Optical density is measured at wavelength of 600 nm with a 1 cm path length using a model Genesys 20 Visible Spectrophotometer (Thermo Scientific). A shake flask is inoculated with the volume of the cell slurry necessary to reach an initial OD600 of 0.1. The inoculation volume is typically around 66 μl. Immediately prior to inoculating, the following materials are added to each 250 ml baffled shake flask: 50 grams of liquified corn mash, 1900 of 500 g/L filter-sterilized urea, and 2.50 of a 100 mg/ml filter sterilized stock of ampicillin. The shake flasks receive a quantity of glucoamylase (Spirizyme Fuel HS™ Novozymes; lot NAPM3771) to achieve a dose of 0.33 AGU/g of Dry Solids is added to the flasks. Glucoamylase activity is measured using the Glucoamylase Activity Assay (described in the Examples section). Duplicate flasks for each strain are incubated at 33.3° C. with shaking in an orbital shaker at 100 rpm for approximately 48 hours. At 48 hours, 1 ml samples are taken and analyzed for ethanol and glucose concentrations in the broth by high performance liquid chromatography with refractive index detector.

### Test 4

Aspects of the disclosure relate to engineered yeast strains that exhibit glycerol reduction of at least 30% by 48 hours, when compared to an unmodified reference strain, under Test 4 conditions, involving evaluating strains in a simultaneous saccharification fermentation (SSF) shake flask assay.

As used here “Test 4 conditions” refers to the following:

Strains are struck to a ScD-ura plate and incubated at 30° C. until single colonies are visible (2-3 days). Cells from the ScD-ura plate are scraped into sterile shake flask medium and the optical density (OD600) is measured. Optical density is measured at wavelength of 600 nm with a 1 cm path length using a model Genesys20 spectrophotometer (Thermo Scientific). A shake flask is inoculated with the cell slurry to reach an initial OD600 of 0.1. Immediately prior to inoculating, 50 mL of shake flask medium is added to a 250 mL baffled shake flask sealed with air-lock containing 4 mls of sterilized canola oil. The shake flask medium consists of 725 g partially hydrolyzed corn starch, 150 g filtered light steep water, 10 g water, 25 g glucose, and 1 g urea. Strains are incubated at 30° C. with shaking in an orbital shake at 100 rpm for 72 hours. Samples are taken and analyzed for metabolite concentrations in the broth during fermentation by HPLC.

In some embodiments, engineered yeast strains described herein produce at least 30% less glycerol than a reference strain. In some embodiments, a reference strain is the control strain Strain 1. In some embodiments, engineered yeast strains described herein produce at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or at least 50% less glycerol than a reference strain by 48 hrs.

### Ethanol Yield

Engineered yeast described herein produce high ethanol concentration. Ethanol concentration can be indicated by a grams per kilogram (g/kg) scale or a grams per liter (g/L) scale.

In some embodiments, the ethanol concentration in the fermentation broth at the end of fermentation is about or at least 10, about or at least 15, about or at least 20, about or at least 25, about or at least 30, about or at least 35, about or at least 40, about or at least 45, about or at least 50, about or at least 55, about or at least 60, about or at least 65, about or at least 70, about or at least 75, about or at least 80, about or at least 85, about or at least 90, about or at least 95, about or at least 100, about or at least 105, about or at least 110, about or at least 115, about or at least 120, about or at least 125, about or at least 130, about or at least 135, about or at least 140, about or at least 145, about or at least 150, about or at least 155, about or at least 160, about or at least 165, about or at least 170, about or at least 175, about or at least 180, (grams per kilogram), including all intermediate values and ranges, or more than 180 g/kg.

In some embodiments, the ethanol concentration in the fermentation broth at the end of fermentation is about or at least 10, about or at least 15, about or at least 20, about or at least 25, about or at least 30, about or at least 35, about or at least 40, about or at least 45, about or at least 50, about or at least 55, about or at least 60, about or at least 65, about or at least 70, about or at least 75, about or at least 80, about or at least 85, about or at least 90, about or at least 95, about or at least 100, about or at least 105, about or at least 110, about or at least 115, about or at least 120, about or at least 125, about or at least 130, about or at least 135, about or at least 140, about or at least 145, about or at least 150, about or at least 155, about or at least 160, about or at least 165, about or at least 170, about or at least 175, about or at least 180 (grams per liter), including all intermediate values and ranges, or more than 180 g/L.

Ethanol mass yield can be calculated by dividing the ethanol concentration by the total glucose consumed. Since glucose can be present as free glucose or tied up in oligomers, one needs to account for both. To determine the total glucose present at the beginning and end of fermentation, a total glucose equivalents measurement (TGE) is determined. The TGE measurement is performed as follows. Glucose is measured with HPLC using RI detection. Separation is completed with a Bio Rad 87H column using a 10 mM H2SO4 mobile phase. An acid hydrolysis is performed in triplicate in 6% (v/v) trifluoroacetic acid at 121° C. for 15 minutes. The resulting glucose after hydrolysis is measured by the same HPLC method. The total glucose equivalents present in each sample is the amount of glucose measured after acid hydrolysis. The total glucose consumed is calculated by subtracting the total glucose equivalents present at the end of fermentation from the total glucose equivalents present at the beginning of the fermentation.

Ethanol yield can be calculated as an increase over a reference yeast strain, for example a reference strain that does not contain one or more of the genetic modifications of engineered yeast strains described herein. In some embodiments, the equation for Ethanol Yield can be defined as: (Ethanol Titer at Time final−Ethanol Titer at Time zero) divided by TGE at Time zero. In some embodiments, ethanol yield is determined using the equation referred to as “Test 3” below.

### Test 3

$$Ethanol Yield ( \% ) = \frac{( Ethanol Titer at {T}_{final} - Ethanol Titer at {T}_{zero} )}{Total Glucose Equivalents at {T}_{zero}} \times 100$$

In some embodiments, the increase in ethanol yield in an engineered strain described herein relative to a reference strain is about or at least 0.05%, about or at least 0.1%, about or at least 0.2%, about or at least 0.3%, about or at least 0.4%, about or at least 0.5%, about or at least 0.6%, about or at least 0.7%, about or at least 0.8%, about or at least 0.9%, about or at least 1%, about or at least 1.1%, about or at least 1.2%, about or at least 1.3%, about or at least 1.4%, about or at least 1.5%, about or at least 1.6%, about or at least 1.7%, about or at least 1.8%, about or at least 1.9%, about or at least 2%, about or at least 2.5%, about or at least 3%, about or at least 3.5%, about or at least 4%, about or at least 4.5%, or about or at least 5%, relative to a reference strain, including all intermediate values and ranges, or more than 5%.

### Expression of Recombinant Nucleic Acids

As one of ordinary skill in the art would be aware, homologous genes for enzymes described herein can be obtained from other species and can be identified by homology searches, for example through a protein BLAST search, available at the National Center for Biotechnology Information (NCBI) internet site (www.ncbi.nlm.nih.gov). Genes can be cloned, for example by PCR amplification and/or restriction digestion, from DNA from any source of DNA which contains the given gene. In some embodiments, a gene is synthetic. Any means of obtaining or synthesizing a gene encoding an enzyme can be used.

The present disclosure relates to the recombinant expression of genes encoding enzymes discussed above, functional modifications and variants thereof, as well as uses relating thereto. Homologs and alleles of the nucleic acids associated with the invention can be identified by conventional techniques. Homologs and alleles will typically share at least 75% nucleotide identity and/or at least 90% amino acid identity to the sequences of nucleic acids and polypeptides, respectively, in some instances will share at least 90% nucleotide identity and/or at least 95% amino acid identity and in still other instances will share at least 95% nucleotide identity and/or at least 99% amino acid identity. The homology can be calculated using various, publicly available software tools developed by NCBI (Bethesda, Md.) that can be obtained through the NCBI internet site. Exemplary tools include the BLAST software, also available at the NCBI internet site (www.ncbi.nlm.nih.gov). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are also contemplated herein.

For example, an alignment can be performed using BLAST (National Center for Biological Information (NCBI) Basic Local Alignment Search Tool) version 2.2.31 software with default parameters. Amino acid % sequence identity between amino acid sequences can be determined using standard protein BLAST with the following default parameters: Max target sequences: 100; Short queries: Automatically adjust parameters for short input sequences; Expect threshold: 10; Word size: 6; Max matches in a query range: 0; Matrix: BLOSUM62; Gap Costs: (Existence: 11, Extension: 1); Compositional adjustments: Conditional compositional score matrix adjustment; Filter: none selected; Mask: none selected. Nucleic acid % sequence identity between nucleic acid sequences can be determined using standard nucleotide BLAST with the following default parameters: Max target sequences: 100; Short queries: Automatically adjust parameters for short input sequences; Expect threshold: 10; Word size: 28; Max matches in a query range: 0; Match/Mismatch Scores: 1, −2; Gap costs: Linear; Filter: Low complexity regions; Mask: Mask for lookup table only. A sequence having an identity score of XX % (for example, 80%) with regard to a reference sequence using the NCBI BLAST version 2.2.31 algorithm with default parameters is considered to be at least XX % identical or, equivalently, have XX % sequence identity to the reference sequence.

The present disclosure also relates to degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the present disclosure embraces degenerate nucleic acids that differ from the biologically isolated nucleic acids in codon sequence due to the degeneracy of the genetic code.

Also disclosed herein are strategies to optimize production of ethanol in a cell. Optimized production of ethanol refers to producing a higher amount of ethanol following an optimization strategy than would be achieved in the absence of the optimization strategy. In some embodiments, optimized production of ethanol involves modifying a gene encoding for an enzyme involved in ethanol production before it is recombinantly expressed in a cell. In some embodiments, the modification involves codon optimization for expression in a cell (e.g., host organism, such as yeast). Codon usage for a variety of organisms can be accessed in databases available to one of ordinary skill in the art, such as the Codon Usage Database (kazusa.or.jp/codon/). Codon optimization, including identification of optimal codons for a variety of organisms, and methods for achieving codon optimization, are familiar to one of ordinary skill in the art and can be achieved using standard methods. It should be appreciated that various codon-optimized forms of any of the nucleic acid and protein sequences described herein can be used in the products and methods disclosed herein.

In some embodiments, production of ethanol in a cell can be optimized through manipulation of enzymes that act in the same pathway as the enzymes described herein (e.g., increase expression of an enzyme or other factor that acts upstream or downstream of a target enzyme such as an enzyme described herein). This could be achieved by over-expressing the upstream or downstream factor using any standard method.

In some embodiments, modifying a gene encoding an enzyme before it is recombinantly expressed in a cell involves making one or more mutations in the gene encoding the enzyme before it is recombinantly expressed in a cell. For example, a mutation can involve a substitution or deletion of a single nucleotide or multiple nucleotides. In some embodiments, a mutation of one or more nucleotides in a gene encoding an enzyme will result in a mutation in the enzyme, such as a substitution or deletion of one or more amino acids.

Additional changes can include increasing copy numbers of the gene components of pathways active in production of ethanol, such as by additional episomal expression. In some embodiments, screening for mutations in components of the production of ethanol, or components of other pathways, that lead to enhanced production of ethanol may be conducted through a random mutagenesis screen, or through screening of known mutations. In some embodiments, shotgun cloning of genomic fragments could be used to identify genomic regions that lead to an increase in production of ethanol, through screening cells or organisms that have these fragments for increased production of ethanol. In some cases one or more mutations may be combined in the same cell or organism.

In some embodiments, the production of ethanol is increased by selecting promoters of various strengths to drive expression of genes. In some embodiments, this may include the selection of high-copy number plasmids, or low or medium-copy number plasmids. The step of transcription termination can also be targeted for regulation of gene expression, through the introduction or elimination of structures such as stem-loops.

Proteins or polypeptides containing the wildtype residues, mutated residues, or codon optimized residues encoded by a gene described herein and isolated nucleic acid molecules encoding the polypeptides are also contemplated herein. As used herein, the terms “protein” and “polypeptide” are used interchangeably and thus the term polypeptide may be used to refer to a full-length polypeptide and may also be used to refer to a fragment of a full-length polypeptide.

In some embodiments described herein, the cell expresses an endogenous copy of one or more of the genes disclosed herein, a recombinant copy of one or more of the genes disclosed herein, or an endogenous copy of one or more of the genes disclosed herein and a recombinant copy of one or more of the genes disclosed herein for increased production of ethanol.

As used herein, the term “overexpression” or “increased expression” refers to an increased level of expression of a gene or a gene product in a cell, cell type or cell state, as compared to a reference cell (e.g., a wildtype cell of the same cell type or a cell of the same cell type that has not been modified, such as genetically modified). For example, in some embodiments, overexpression of one or more genes encoding a GapN enzyme and a glucoamylase enzyme in an engineered cell results in higher production of ethanol relative to a reference cell, such as a wildtype cell, that does not overexpress one or more genes encoding a gapN enzyme and a glucoamylase enzyme. In some embodiments, overexpression or increased expression of a gene in an engineered cell described herein is achieved by recombinantly expressing an endogenous gene to thereby increase expression of the gene. In some embodiments, overexpression or increased expression of a gene in an engineered cell described herein is achieved by recombinantly expressing a gene that is not endogenous to the engineered cell to thereby increase expression of the gene.

The term “exogenous” as used herein means any material that originated outside the microorganism of interest. For example, the term “exogenous” can be applied to genetic material not present in the native form of a particular organism prior to genetic modification (i.e., such exogenous genetic material could also be referred to as heterologous), or it can also be applied to an enzyme or other protein that does not originate from a particular organism.

As disclosed herein and understood by one of ordinary skill in the art, the activity or expression of one or more genes and gene products can be reduced, attenuated or eliminated in several ways, including by reducing expression of the relevant gene, disrupting the relevant gene, introducing one or more mutations in the relevant gene that results in production of a protein with reduced, attenuated or eliminated enzymatic activity, and/or use of specific inhibitors to reduce, attenuate or eliminate the enzymatic activity, including using nucleic acids, such as micro-RNA (miRNA) or small interfering RNA (siRNA), etc.

In some embodiments, one or more of the genes disclosed herein is expressed using a vector. In some embodiments, a vector replicates autonomously in the cell. In other embodiments, the vector integrates into the genome of the cell. A vector can contain one or more endonuclease restriction sites that are cut by a restriction endonuclease to insert and ligate a nucleic acid containing a gene described herein to produce a recombinant vector that is able to replicate in a cell. Vectors are typically composed of DNA, although RNA vectors are also available.

Cloning vectors include, but are not limited to: plasmids, fosmids, phagemids, virus genomes and artificial chromosomes. As used herein, the terms “expression vector” or “expression construct” refer to a nucleic acid construct, generated recombinantly or synthetically, with a series of specified nucleic acid elements that permit transcription of a particular nucleic acid in a host cell (e.g., microbe), such as a yeast cell. In some embodiments, the nucleic acid sequence of a gene described herein is inserted into a cloning vector such that it is operably joined to regulatory sequences and, in some embodiments, expressed as an RNA transcript.

In some embodiments, the vector contains one or more markers to identify cells transformed or transfected with the recombinant vector. Markers include, for example, genes encoding proteins which increase or decrease resistance or sensitivity to compounds (e.g., antibiotics), genes encoding enzymes (e.g., β-galactosidase, luciferase or alkaline phosphatase) whose activities are detectable by standard assays known to one of ordinary skill in the art, and genes which visibly affect the phenotype of transformed or transfected cells, hosts, colonies or plaques (e.g., encoding fluorescent proteins such as green fluorescent protein). In certain embodiments, the marker is an amdS marker or a URA3 marker.

A coding sequence and a regulatory sequence are said to be “operably joined” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined if induction of a promoter in the 5′ regulatory sequence transcribes the coding sequence and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein. Thus, a promoter region is operably joined to a coding sequence if the promoter region transcribes the coding sequence and the transcript can be translated into the protein or polypeptide of interest.

In some embodiments, the nucleic acid encoding any of the proteins described herein is under the control of regulatory sequences (e.g., enhancer sequences). In some embodiments, a nucleic acid is expressed under the control of a promoter. The promoter can be a native promoter (e.g., the promoter of the gene in its endogenous context, which provides normal regulation of expression of the gene). Alternatively, a promoter can be a promoter that is different from the native promoter of the gene, e.g., the promoter is different from the promoter of the gene in its endogenous context. In some embodiments, the promoter of a gene that increases the production of ethanol in a cell, or decreases production of glycerol in a cell, is modified. A “modified promoter” refers to a promoter whose nucleotide sequence has been altered. In some embodiments, the modified promoter has increased or decreased transcriptional activity relative to an unmodified promoter. In some embodiments, a modified promoter is obtained by nucleotide deletion(s), insertion(s) or mutation(s), or any combination thereof. In some embodiments, a promoter is altered, for instance, by homologous recombination, gene targeting, knockout, knock in, site-directed mutagenesis, or artificial zinc finger nuclease-mediated strategies, by a random or quasi-random event (e.g., irradiation or non-targeted nucleotide integration and subsequent selection). Other methods for modifying a promoter to increase the transcriptional activity of the promoter known to one of ordinary skill in the art are also contemplated herein.

As used herein, a “heterologous promoter” is a promoter that is not naturally or normally associated with or that does not naturally or normally control transcription of a DNA sequence to which it is operably joined. In some embodiments, a nucleic acid sequence or a gene described herein is under the control of a heterologous promoter.

In some embodiments, the promoter is a eukaryotic promoter. Non-limiting examples of eukaryotic promoters include TDH3, PGK1, PKC1, TDH2, PYK1, TPI1, AT1, CMV, EF1a, SV40, Ubc, human beta actin, CAG, TRE, UAS, Ac5, Polyhedrin, CaMKIIa, GAL1, GAL10, TEF1, GDS, ADH1, CaMV35S, Ubi, H1, U6, and TEF1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, PL. Non-limiting examples of bacterial promoters include Pbad, PmgrB, Ptrc2, Plac/ara, Ptac, Pm.

In some embodiments, the promoter is an inducible promoter. As used herein, an “inducible promoter” is a promoter controlled by the presence or absence of a molecule. Non-limiting examples of inducible promoters include chemically-regulated promoters and physically-regulated promoters. For chemically-regulated promoters, the transcriptional activity is regulated by one or more compounds, such as alcohol, tetracycline, galactose, a steroid, a metal, or other compounds. For physically-regulated promoters, transcriptional activity is regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used herein, a “constitutive promoter” refers to an unregulated promoter that allows continuous transcription of a gene. Non-limiting examples of a constitutive promoter includes CP1, CMV, EF1a, SV40, PGK1, Ubc, human beta actin, CAG, Ac5, polyhedrin, TEF1, GDS, CaM35S, Ubi, H1, and U6. Other inducible promoters or constitutive promoters known to one of ordinary skill in the art are also contemplated herein.

In some embodiments, the cell is engineered by the introduction of a heterologous nucleic acid (e.g., DNA and/or RNA). That heterologous nucleic acid can be placed under operable control of transcriptional elements to permit the expression of the heterologous DNA or RNA in an engineered cell described herein. Heterologous expression of genes for production of ethanol is demonstrated in the Example section using S. cerevisiae. Production of ethanol using novel methods described herein in other cells, including other fungal cells is also contemplated herein.

The precise nature of the regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. The vectors disclosed herein may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described herein in a heterologous organism is within the ability and discretion of one of ordinary skill in the art.

Expression vectors containing the necessary elements for expression are commercially available and known to one of ordinary skill in the art (see, e.g., Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York, 2010).

In some embodiments, one or more of the recombinantly expressed genes disclosed herein are introduced into an engineered cell using standard methods known to one of ordinary skill in the art. Non-limiting examples include transformation (e.g., chemical transformation, electroporation, etc.), transduction, particle bombardment, etc. In some embodiments, one or more of the genes disclosed herein are integrated into the genome of the cell.

### Nucleic Acid and Protein Sequences

GapN gene and amino acid sequences are well known to one of ordinary skill in the art. Non-limiting examples of GapN gene and protein sequences include:

Codon-optimized GAPN DNA sequence from Bacillus
cereus (SEQ ID NO: 45):
ATGACAACATCAAATACCTACAAATTCTATCTAAACGGTGAATGGAGAGAA
TCTTCCTCTGGAGAAACTATTGAGATACCATCACCATACTTACATGAAGTG
ATCGGACAGGTTCAAGCAATCACTAGAGGAGAGGTTGACGAAGCGATTGCT
AGCGCTAAGGAAGCACAGAAATCTTGGGCTGAGGCATCTCTACAAGATAGA
GCTAAGTACTTGTACAAATGGGCAGATGAATTGGTAAACATGCAAGACGAA
ATCGCCGATATCATCATGAAGGAAGTGGGCAAGGGTTACAAAGACGCTAAA
AAGGAGGTTGTTAGAACCGCCGATTTCATCAGATACACCATTGAAGAGGCA
CTCCATATGCACGGTGAATCCATGATGGGCGATTCATTTCCTGGTGGAACA
AAATCTAAGCTAGCAATAATCCAAAGAGCGCCTCTGGGTGTAGTCTTAGCC
ATCGCTCCATTCAATTACCCTGTAAACCTTTCTGCTGCAAAATTGGCACCA
GCCTTAATTATGGGTAACGCTGTGATATTCAAGCCAGCAACTCAGGGTGCT
ATTTCCGGCATCAAAATGGTTGAAGCTTTGCATAAGGCTGGTTTGCCAAAG
GGTTTGGTTAACGTTGCCACAGGTAGAGGTAGCGTCATAGGCGATTATTTG
GTCGAACACGAAGGGATAAACATGGTTTCCTTCACCGGTGGCACTAACACT
GGTAAGCATTTAGCAAAAAAGGCCTCAATGATTCCATTAGTCTTGGAACTT
GGTGGCAAAGATCCAGGCATCGTTCGTGAAGATGCAGACCTACAAGATGCT
GCGAATCATATCGTATCTGGTGCGTTCAGTTACTCAGGGCAGAGATGTACA
GCCATTAAGAGAGTCCTTGTTCATGAAAATGTTGCTGATGAACTGGTATCA
TTGGTTAAGGAACAAGTGGCAAAGCTTTCTGTGGGATCACCAGAGCAAGAT
TCAACAATTGTTCCTCTGATTGACGATAAGTCCGCTGATTTTGTTCAGGGT
TTAGTGGACGATGCAGTCGAAAAGGGCGCTACAATTGTCATTGGGAACAAG
AGAGAACGTAACCTAATCTACCCAACATTGATTGATCACGTCACAGAGGAA
ATGAAAGTTGCCTGGGAGGAACCATTCGGTCCTATTCTTCCAATTATTAGA
GTTAGTAGCGACGAGCAAGCTATTGAAATTGCAAATAAGAGTGAGTTCGGA
TTACAAGCTTCTGTGTTTACCAAAGACATAAACAAGGCATTCGCAATCGCA
AATAAGATTGAGACTGGTTCAGTGCAAATCAACGGTAGAACAGAGAGAGGA
CCAGATCACTTTCCTTTTATCGGGGTTAAGGGATCTGGGATGGGTGCCCAA
GGCATCAGAAAGTCTTTGGAATCTATGACTAGAGAAAAAGTTACTGTCTTA
AATCTCGTATGA
GapN protein sequence from Bacillus cereus
(SEQ ID NO: 42):
MTTSNTYKFYLNGEWRESSSGETIEIPSPYLHEVIGQVQAITRGEVDEAIA
SAKEAQKSWAEASLQDRAKYLYKWADELVNMQDEIADIIMKEVGKGYKDAK
KEVVRTADFIRYTIEEALHMHGESMMGDSFPGGTKSKLAIIQRAPLGVVLA
IAPFNYPVNLSAAKLAPALIMGNAVIFKPATQGAISGIKMVEALHKAGLPK
GLVNVATGRGSVIGDYLVEHEGINMVSFTGGTNTGKHLAKKASMIPLVLEL
GGKDPGIVREDADLQDAANHIVSGAFSYSGQRCTAIKRVLVHENVADELVS
LVKEQVAKLSVGSPEQDSTIVPLIDDKSADFVQGLVDDAVEKGATIVIGNK
RERNLIYPTLIDHVTEEMKVAWEEPFGPILPIIRVSSDEQAIEIANKSEFG
LQASVFTKDINKAFAIANKIETGSVQINGRTERGPDHFPFIGVKGSGMGAQ
GIRKSLESMTREKVTVLNLV

Glucoamylase gene and protein sequences are well known to one of ordinary skill in the art. Non-limiting examples of glucoamylase gene and protein sequences include:

Codon-optimized glucoamylase DNA sequence (GLA1
gene) from Saccharomycopsis fibuligera
(SEQ ID NO: 46)
ATGATTAGATTAACCGTATTCCTCACTGCAGTTTTTGCAGCAGTCGCTTCC
TGTGTTCCAGTTGAATTGGATAAGAGAAATACAGGCCATTTCCAAGCATAT
TCTGGTTACACCGTAGCTAGATCAAACTTTACTCAATGGATTCACGAGCAA
CCAGCCGTATCATGGTACTATTTGCTTCAGAATATAGACTATCCAGAAGGA
CAATTCAAGTCTGCCAAGCCAGGGGTCGTTGTGGCTTCCCCTTCTACATCC
GAACCTGATTACTTCTACCAATGGACTAGAGATACTGCTATCACCTTCTTG
TCACTTATCGCGGAAGTTGAGGATCATTCTTTTTCAAATACTACACTAGCC
AAGGTGGTTGAATACTACATCTCTAATACTTACACATTACAAAGAGTTTCC
AACCCATCTGGTAACTTCGACAGTCCAAATCACGACGGTTTGGGAGAACCA
AAGTTTAATGTTGATGATACAGCTTATACTGCATCTTGGGGTAGACCACAA
AATGATGGCCCAGCGTTGAGAGCATACGCAATTTCAAGATACCTTAACGCA
GTAGCAAAACACAACAACGGTAAGTTACTGCTCGCTGGACAAAACGGTATT
CCTTACTCTTCAGCTTCTGATATCTACTGGAAGATTATCAAGCCAGATCTT
CAACATGTGTCAACCCATTGGTCTACATCTGGTTTTGATTTGTGGGAAGAG
AATCAGGGAACACATTTCTTTACTGCGTTGGTCCAGCTAAAAGCACTTAGT
TACGGCATTCCTTTAAGTAAGACCTACAACGATCCTGGTTTCACTAGTTGG
CTAGAAAAGCAAAAGGATGCTTTAAACTCTTATATCAACAGCTCTGGTTTC
GTAAACTCTGGCAAAAAGCATATAGTGGAGAGCCCTCAACTATCTTCAAGA
GGAGGGTTGGATAGCGCCACATACATTGCAGCCTTAATCACACATGATATT
GGCGACGACGACACTTACACACCTTTCAACGTTGACAACTCCTATGTCTTG
AACTCACTGTATTACCTTCTAGTCGATAACAAAAACCGTTACAAAATCAAT
GGTAACTACAAGGCCGGTGCTGCTGTTGGTAGATACCCAGAGGATGTTTAC
AACGGTGTTGGGACATCAGAAGGCAATCCATGGCAATTAGCTACAGCCTAC
GCCGGCCAAACATTTTACACACTGGCTTACAACTCATTGAAAAACAAAAAA
AACTTAGTGATTGAAAAGTTGAACTACGACCTCTACAATTCTTTCATAGCA
GATTTATCCAAGATCGATAGTTCTTACGCATCAAAAGACTCCTTGACTTTG
ACCTACGGTTCTGACAACTACAAAAACGTCATAAAGTCACTATTACAGTTT
GGAGATTCATTCCTGAAGGTCTTGCTCGATCACATTGATGATAATGGACAA
TTAACAGAAGAGATCAATAGATACACAGGGTTCCAGGCTGGTGCTGTTAGT
TTGACATGGTCCTCTGGTTCATTACTTTCAGCAAACCGTGCGAGAAATAAG
TTGATTGAACTATTGTAG
Codon-optimized glucoamylase DNA sequence (GLA1
gene) from Saccharomycopsis fibuligera
(SEQ ID NO: 47)
ATGATCAGACTTACAGTTTTCCTAACAGCCGTTTTCGCCGCCGTTGCATCA
TGTGTCCCAGTAGAATTGGATAAGAGAAACACCGGCCATTTCCAAGCATAT
TCAGGATACACCGTTGCACGTTCTAATTTCACACAATGGATTCATGAGCAG
CCTGCTGTGTCCTGGTACTACTTATTACAAAACATTGATTATCCTGAGGGA
CAATTCAAGTCAGCGAAACCAGGCGTTGTGGTTGCTTCTCCATCCACTTCA
GAACCAGACTACTTCTACCAGTGGACCCGTGACACAGCAATAACTTTCTTA
TCTTTGATAGCAGAAGTAGAAGATCACTCATTTTCAAATACAACTCTAGCT
AAGGTTGTCGAATACTACATCTCTAACACATACACCCTACAAAGAGTTTCT
AACCCATCTGGTAATTTCGATAGCCCAAATCACGATGGTCTGGGTGAACCA
AAGTTCAACGTTGACGACACTGCTTACACTGCATCATGGGGCAGACCTCAA
AACGACGGTCCAGCCTTAAGAGCTTACGCGATCTCAAGATATTTGAACGCA
GTTGCCAAGCATAACAACGGTAAGCTATTGCTCGCGGGTCAAAATGGTATT
CCTTACTCATCTGCATCAGATATCTACTGGAAGATTATCAAGCCAGATTTA
CAACATGTAAGTACTCACTGGAGTACATCTGGTTTTGACTTATGGGAAGAG
AATCAAGGTACACATTTCTTTACTGCACTTGTCCAGTTAAAAGCTCTTTCA
TACGGTATACCTTTGTCTAAGACATATAACGATCCAGGATTTACTTCTTGG
TTGGAAAAGCAGAAGGATGCCTTGAACTCTTACATCAATTCCAGCGGCTTC
GTCAACTCCGGGAAAAAGCACATTGTCGAATCTCCTCAATTATCTAGTAGA
GGGGGTCTTGATAGCGCTACTTACATCGCTGCTCTAATTACACATGATATT
GGTGATGATGATACATACACTCCTTTTAACGTAGATAATTCTTATGTGCTG
AACTCTTTATACTATCTGCTTGTAGACAACAAAAACAGATACAAGATCAAC
GGGAACTACAAAGCAGGAGCTGCAGTTGGTAGATACCCAGAAGATGTGTAC
AATGGAGTGGGAACCTCAGAGGGAAACCCATGGCAATTGGCGACAGCATAC
GCCGGCCAAACCTTTTACACACTGGCTTACAATTCTCTCAAAAACAAAAAA
AATTTGGTTATTGAGAAGTTGAATTACGATCTATACAACTCCTTTATAGCT
GACTTAAGTAAGATTGACTCCTCTTACGCTTCTAAGGATTCATTGACATTG
ACCTACGGCTCAGATAACTACAAAAATGTCATTAAGTCACTTTTACAATTC
GGGGATTCTTTCTTGAAAGTCTTGTTGGACCATATTGATGATAATGGTCAG
CTAACAGAGGAAATCAACAGATATACAGGTTTTCAAGCTGGCGCAGTTTCC
CTCACTTGGAGTAGTGGTTCACTCTTATCTGCAAACAGAGCCAGAAACAAG
TTGATCGAATTGCTTTAG
Codon-optimized glucoamylase DNA sequence (GLA1
gene) from Saccharomycopsis fibuligera
(SEQ ID NO: 48)
ATGATCAGACTTACTGTTTTCCTCACAGCCGTTTTTGCAGCAGTAGCTTCT
TGTGTTCCAGTTGAATTGGATAAGAGAAATACAGGTCATTTCCAAGCTTAC
TCTGGTTACACTGTGGCTAGATCTAACTTCACACAATGGATTCATGAACAG
CCTGCCGTGAGTTGGTACTATTTGCTACAAAACATTGATTACCCTGAGGGT
CAATTCAAATCAGCTAAGCCAGGTGTTGTTGTCGCGAGCCCATCAACTTCT
GAACCAGATTACTTCTACCAATGGACTAGAGATACCGCAATAACCTTCTTA
TCTCTAATCGCAGAGGTAGAAGATCACTCTTTTTCAAATACTACCCTGGCA
AAAGTGGTCGAGTACTACATCTCAAACACATACACCTTGCAGAGAGTCTCA
AACCCATCAGGAAACTTCGATTCTCCTAATCATGACGGCTTAGGAGAACCA
AAGTTTAATGTTGACGATACCGCTTATACTGCATCTTGGGGTAGACCACAG
AATGATGGCCCTGCCTTACGTGCATACGCCATTTCCAGATATCTCAACGCT
GTAGCGAAGCACAACAACGGTAAGCTGCTTTTAGCTGGTCAAAATGGGATA
CCATACTCTTCCGCTTCAGACATTTACTGGAAGATTATCAAACCAGACTTG
CAGCATGTCAGTACACATTGGTCAACTTCTGGTTTTGATTTGTGGGAAGAG
AACCAAGGCACTCACTTCTTTACAGCCTTGGTTCAACTAAAGGCATTGTCT
TACGGAATCCCTTTGTCCAAGACATACAATGATCCTGGATTCACTAGTTGG
CTAGAAAAGCAAAAGGATGCACTGAACTCATACATTAACAGTTCAGGCTTT
GTGAACTCCGGTAAAAAGCATATTGTTGAAAGCCCACAACTATCTAGCAGA
GGTGGTTTAGATTCTGCAACCTACATAGCAGCCTTGATCACACACGACATT
GGGGATGACGATACATACACACCATTCAACGTCGACAATTCATACGTTTTG
AATAGCTTATACTACCTACTGGTAGATAACAAAAACAGATATAAGATCAAT
GGCAACTACAAGGCCGGTGCTGCCGTAGGAAGATACCCTGAAGATGTCTAC
AACGGAGTTGGTACATCAGAAGGTAACCCATGGCAATTAGCAACAGCATAT
GCGGGCCAGACATTTTACACTTTGGCTTACAATTCATTGAAAAACAAAAAA
AATTTAGTGATAGAAAAGCTTAACTATGACCTTTACAACTCTTTCATTGCC
GATTTATCCAAGATTGATTCCTCCTACGCATCAAAGGACTCCTTGACACTT
ACATACGGTTCTGACAACTACAAAAATGTTATCAAGTCTCTCTTGCAATTT
GGTGATTCTTTCTTGAAGGTTTTACTCGATCATATCGATGATAATGGTCAA
CTAACTGAGGAAATCAACAGATACACTGGGTTCCAAGCTGGAGCTGTCTCT
TTAACATGGAGTTCAGGGAGTTTGTTATCTGCTAACAGAGCGCGTAACAAA
CTTATTGAGCTTCTGTAG
Codon-optimized glucoamylase DNA sequence (GLA1
gene) from Saccharomycopsis fibuligera
(SEQ ID NO: 49)
ATGATTAGATTAACAGTATTTCTTACAGCCGTTTTCGCAGCCGTCGCATCC
TGTGTTCCAGTAGAATTAGATAAGCGTAATACAGGACATTTTCAAGCTTAC
TCTGGCTATACAGTTGCGAGATCTAACTTTACACAATGGATTCACGAACAG
CCAGCAGTTTCTTGGTACTATTTGCTCCAAAACATCGACTACCCTGAAGGC
CAATTCAAGTCTGCAAAGCCAGGAGTGGTCGTCGCTTCTCCTAGTACTTCA
GAACCAGATTACTTCTACCAGTGGACAAGAGACACTGCTATTACCTTCCTG
AGCTTAATCGCTGAAGTTGAAGATCACTCTTTTTCTAATACAACACTGGCC
AAAGTAGTTGAGTACTACATCTCTAACACTTACACTCTACAAAGAGTGTCA
AACCCTTCTGGGAACTTCGACAGCCCAAACCATGATGGTTTGGGGGAGCCA
AAATTCAACGTTGATGATACAGCCTACACCGCATCTTGGGGTAGACCACAA
AACGACGGACCAGCTTTAAGAGCATACGCAATATCTCGTTACCTTAATGCT
GTTGCAAAGCACAATAATGGAAAGTTGTTGTTGGCTGGTCAAAACGGTATT
CCTTACTCTTCAGCATCTGATATCTACTGGAAGATTATCAAGCCAGATCTT
CAACACGTATCCACACATTGGTCAACCTCCGGCTTCGATTTATGGGAGGAA
AATCAGGGTACACATTTCTTCACCGCTCTAGTGCAATTGAAGGCTTTGAGT
TACGGCATTCCATTGTCTAAGACTTACAACGATCCTGGTTTCACCTCATGG
CTTGAAAAGCAGAAGGATGCCCTGAATAGCTACATCAACTCATCTGGTTTT
GTTAACTCAGGGAAAAAGCATATAGTTGAATCCCCACAACTATCATCAAGA
GGAGGTTTAGACTCCGCCACATACATTGCTGCCTTGATTACACATGATATT
GGGGATGATGACACATATACTCCATTTAACGTCGATAACAGTTATGTCCTT
AATTCCTTATACTATTTGTTGGTCGATAACAAAAATAGATACAAAATCAAC
GGCAACTACAAGGCTGGCGCAGCGGTGGGTAGATACCCTGAGGATGTTTAC
AATGGTGTAGGTACATCTGAAGGCAATCCATGGCAATTAGCGACTGCTTAC
GCTGGACAAACTTTCTACACACTTGCGTACAACTCATTGAAAAACAAAAAA
AACCTAGTCATTGAAAAGTTGAATTACGATCTGTACAACTCTTTCATCGCA
GACCTATCAAAGATTGACTCATCTTATGCAAGTAAAGATTCACTAACTTTA
ACCTACGGTAGTGATAACTACAAAAACGTTATCAAGTCTTTACTCCAGTTT
GGTGATTCATTCTTGAAGGTGTTGTTAGATCATATAGACGACAATGGTCAA
CTCACAGAGGAGATAAACAGATACACTGGTTTTCAAGCAGGAGCTGTTTCA
CTTACTTGGTCAAGTGGTTCTTTGCTTTCCGCCAACAGAGCCAGAAACAAG
CTCATCGAATTACTATAG
Glucoamylase protein sequence (GLA1 protein) from
Saccharomycopsis fibuligera
(SEQ ID NO: 38)
MIRLTVFLTAVFAAVASCVPVELDKRNTGHFQAYSGYTVARSNFTQWIHEQ
PAVSWYYLLQNIDYPEGQFKSAKPGVVVASPSTSEPDYFYQWTRDTAITFL
SLIAEVEDHSFSNTTLAKVVEYYISNTYTLQRVSNPSGNFDSPNHDGLGEP
KFNVDDTAYTASWGRPQNDGPALRAYAISRYLNAVAKHNNGKLLLAGQNGI
PYSSASDIYWKIIKPDLQHVSTHWSTSGFDLWEENQGTHFFTALVQLKALS
YGIPLSKTYNDPGFTSWLEKQKDALNSYINSSGFVNSGKKHIVESPQLSSR
GGLDSATYIAALITHDIGDDDTYTPFNVDNSYVLNSLYYLLVDNKNRYKIN
GNYKAGAAVGRYPEDVYNGVGTSEGNPWQLATAYAGQTFYTLAYNSLKNKK
NLVIEKLNYDLYNSFIADLSKIDSSYASKDSLTLTYGSDNYKNVIKSLLQF
GDSFLKVLLDHIDDNGQLTEEINRYTGFQAGAVSLTWSSGSLLSANRARNK
LIELL
Codon-optimized glucoamylase DNA sequence (amyA
gene) from Rhizopus oryzae
(SEQ ID NO: 50)
ATGAAGTTCATTTCCACTTTCTTGACCTTCATTTTGGCTGCTGTCTCTGTC
ACCGCTGCATCTATTCCATCTAGTGCATCTGTACAATTGGACTCCTACAAT
TACGATGGTTCCACATTTTCCGGCAAGATTTATGTCAAAAACATCGCTTAC
TCTAAAAAGGTTACTGTTGTGTACGCAGACGGTTCTGACAACTGGAACAAT
AACGGCAACACTATTGCTGCATCATTTTCAGGCCCAATCTCTGGATCAAAT
TACGAATACTGGACATTCTCAGCATCAGTGAAGGGCATAAAGGAGTTCTAC
ATCAAATACGAAGTTTCAGGTAAGACATATTACGACAATAACAACTCTGCA
AACTACCAAGTCTCAACTTCTAAACCTACTACAACTACTGCAGCTACAACC
ACAACTACAGCTCCATCAACTTCTACAACAACCCGTCCATCTAGTTCAGAG
CCTGCCACCTTCCCTACTGGTAATTCTACCATCAGCTCTTGGATCAAAAAG
CAGGAAGATATTTCCAGATTCGCTATGCTTAGAAACATCAACCCACCTGGT
TCTGCCACAGGGTTTATCGCCGCATCACTCTCTACCGCTGGTCCAGATTAC
TACTACGCGTGGACAAGAGATGCCGCTTTGACATCTAACGTTATCGTTTAC
GAATACAACACCACATTGTCTGGGAATAAGACAATTCTAAACGTACTTAAG
GATTACGTCACATTCAGTGTTAAGACACAGTCTACTTCAACAGTTTGTAAT
TGCCTTGGTGAACCAAAGTTCAATCCAGACGGCAGTGGTTACACAGGTGCT
TGGGGTAGACCTCAAAATGATGGTCCTGCAGAAAGAGCGACTACATTTGTT
CTGTTTGCCGACAGCTACTTGACTCAAACTAAGGATGCCTCATACGTCACT
GGTACATTAAAGCCAGCAATTTTCAAAGATCTCGATTACGTTGTTAACGTC
TGGAGTAACGGATGTTTCGATTTATGGGAGGAGGTGAACGGAGTTCATTTC
TACACCCTTATGGTTATGAGAAAAGGGCTATTGTTGGGGGCTGATTTCGCG
AAGAGAAACGGTGACTCAACTAGAGCCTCAACTTACTCTTCTACTGCTTCC
ACAATTGCTAACAAGATATCAAGTTTCTGGGTTAGCTCAAACAACTGGGTG
CAAGTATCCCAATCTGTCACAGGAGGTGTAAGTAAAAAGGGGTTAGACGTT
AGCACCCTGTTAGCTGCGAATCTAGGATCAGTCGATGATGGATTTTTCACT
CCAGGTTCTGAAAAGATATTAGCTACAGCTGTGGCAGTCGAAGATTCCTTT
GCCAGTCTATACCCAATCAACAAAAACCTTCCATCATACTTGGGGAACGCT
ATTGGAAGATACCCTGAAGATACATACAACGGTAATGGTAACTCACAAGGC
AATCCTTGGTTTCTGGCGGTTACCGGCTACGCAGAGTTGTACTATAGAGCA
ATTAAGGAATGGATTTCTAATGGAGGCGTTACAGTGTCCTCTATCTCATTG
CCATTTTTCAAAAAGTTCGATAGCTCTGCAACATCCGGTAAAAAGTACACC
GTAGGTACTTCTGACTTCAACAATTTAGCACAAAACATTGCTCTTGCTGCA
GATCGTTTCCTATCTACTGTACAACTCCATGCACCAAACAATGGTTCATTA
GCAGAGGAATTTGATAGAACAACAGGTTTTTCTACCGGCGCTAGAGATTTA
ACATGGTCCCACGCCTCATTGATAACAGCATCCTATGCCAAAGCCGGTGCT
CCAGCTGCATAA
Codon-optimized glucoamylase DNA sequence (amyA
gene) from Rhizopus oryzae
(SEQ ID NO: 51)
ATGAAGTTTATCTCCACGTTTTTAACCTTTATCCTAGCAGCTGTCAGCGTC
ACCGCCGCATCAATTCCGAGTTCAGCATCTGTACAACTTGACTCTTACAAT
TACGATGGCAGCACTTTCTCAGGGAAAATTTATGTGAAAAACATAGCATAT
AGTAAGAAGGTTACCGTGGTATATGCAGACGGTTCTGATAATTGGAATAAT
AATGGAAACACTATTGCCGCCAGTTTTTCCGGCCCAATTTCTGGTTCCAAT
TACGAGTATTGGACCTTTTCTGCATCAGTAAAAGGCATCAAGGAATTCTAT
ATTAAGTACGAAGTTTCAGGTAAGACATATTACGATAACAATAACTCAGCA
AATTATCAAGTCTCTACATCTAAGCCCACAACAACAACTGCTGCTACCACC
ACTACAACCGCTCCTTCTACCAGCACCACTACCAGACCAAGCTCTAGTGAA
CCGGCTACCTTTCCTACCGGAAACAGTACCATCTCAAGCTGGATCAAAAAG
CAAGAGGACATAAGTCGTTTTGCTATGTTGAGGAACATTAATCCTCCAGGA
TCCGCGACCGGTTTCATTGCAGCATCACTAAGTACTGCCGGGCCTGATTAT
TATTATGCTTGGACTAGAGACGCTGCATTAACATCAAACGTGATTGTTTAT
GAATATAATACGACCCTTTCCGGTAATAAAACGATCTTGAACGTATTAAAA
GACTATGTGACCTTTAGTGTGAAGACCCAATCTACATCTACAGTGTGTAAT
TGTTTGGGAGAACCTAAATTCAATCCAGACGGTTCTGGGTACACTGGTGCC
TGGGGTAGACCTCAAAACGACGGTCCAGCAGAAAGAGCAACAACCTTTGTT
CTATTTGCTGACTCTTATTTAACGCAAACAAAGGACGCCTCATATGTTACA
GGGACCCTAAAACCAGCAATTTTCAAAGACTTGGATTATGTTGTTAATGTT
TGGAGCAACGGATGTTTTGACTTGTGGGAGGAGGTTAACGGTGTACACTTT
TATACATTGATGGTGATGAGAAAAGGGTTGCTATTGGGAGCAGATTTCGCT
AAAAGAAATGGTGATTCTACAAGAGCGAGCACATATAGTAGCACCGCTTCA
ACAATCGCCAATAAAATCTCATCTTTCTGGGTATCTAGCAACAACTGGGTA
CAAGTTTCCCAAAGTGTTACCGGCGGTGTGTCCAAAAAGGGTTTAGACGTT
AGCACACTTCTAGCTGCTAATTTGGGTAGCGTTGATGACGGGTTTTTTACT
CCAGGTAGTGAGAAGATACTGGCAACCGCGGTGGCGGTTGAAGACAGCTTT
GCTTCATTGTATCCTATAAATAAAAATCTGCCCTCTTATCTGGGTAATGCA
ATTGGCAGATACCCAGAAGATACCTACAATGGTAATGGTAATTCCCAGGGG
AACCCATGGTTTTTGGCTGTTACAGGCTACGCAGAACTTTATTACCGTGCA
ATCAAGGAATGGATTTCAAATGGCGGCGTCACTGTCAGTAGTATAAGTTTG
CCCTTTTTTAAGAAATTTGATTCCTCAGCAACGTCTGGTAAAAAATACACC
GTAGGTACTAGTGATTTCAATAATTTGGCCCAAAATATTGCGCTTGCTGCT
GACAGGTTTCTTAGTACCGTTCAGTTGCACGCTCCAAATAATGGCTCATTG
GCTGAAGAATTTGATCGTACGACAGGTTTCTCCACTGGTGCTAGGGATTTG
ACTTGGAGTCATGCCTCCTTAATCACAGCAAGCTATGCTAAAGCTGGTGCA
CCTGCTGCTTAG
Glucoamylase protein sequence (amyA protein) from
Rhizopus oryzae
(SEQ ID NO: 39)
MKFISTFLTFILAAVSVTAASIPSSASVQLDSYNYDGSTFSGKIYVKNIAY
SKKVTVVYADGSDNWNNNGNTIAASFSGPISGSNYEYWTFSASVKGIKEFY
IKYEVSGKTYYDNNNSANYQVSTSKPTTTTAATTTTTAPSTSTTTRPSSSE
PATFPTGNSTISSWIKKQEDISRFAMLRNINPPGSATGFIAASLSTAGPDY
YYAWTRDAALTSNVIVYEYNTTLSGNKTILNVLKDYVTFSVKTQSTSTVCN
CLGEPKFNPDGSGYTGAWGRPQNDGPAERATTFVLFADSYLTQTKDASYVT
GTLKPAIFKDLDYVVNVWSNGCFDLWEEVNGVHFYTLMVMRKGLLLGADFA
KRNGDSTRASTYSSTASTIANKISSFWVSSNNWVQVSQSVTGGVSKKGLDV
STLLAANLGSVDDGFFTPGSEKILATAVAVEDSFASLYPINKNLPSYLGNA
IGRYPEDTYNGNGNSQGNPWFLAVTGYAELYYRAIKEWISNGGVTVSSISL
PFFKKFDSSATSGKKYTVGTSDFNNLAQNIALAADRFLSTVQLHAPNNGSL
AEEFDRTTGFSTGARDLTWSHASLITASYAKAGAPAA
Codon-optimized glucoamylase gene sequence (amyA
protein) from Rhizopus delemar
(SEQ ID NO: 52)
ATGCAGTGTTCAATTGCATTAAAGGTTTCATTCTTTTTGGTCTATCATATT
TAGTTTGTTGGTGTCAGCGCATTATTCCATTTCAGCATTGTACAATTAGAT
CTACAATTAGAGGTTACATTCAGGGAAAGATTTAGTGAAAAATATTGGTAC
AGCAAAAAAGTAACTGTTATTATGCGAGGATCAGATAATGGAACAACAATG
GAAACATATGTGCCAGTTATTGCACCAATTTCAGGTTTAATAGAATATTGG
ACATTTCAGCTCCATCAATGGCATTAAGGAATTTACATAAAGTAGAAGTTT
CGGTAAGATTATAGATAACAACAATTCTGCAAATATCAAGTATCAACATCA
AAATATACACAGCACAGTACAATACAATGCACTTCAACATTACCACAACCC
CACCATCTTTAGGAACCAGTACATTCCCAATGGCAATTTATATTTTAGTTG
GATCAAAAAACAAGAGGGTATTTCCAGATTGCAATGTTGAGAAACATAAAT
CCACCAGGATCAGCAATGGATTCATGCAGTTCTTTGTCCACAGGGGGCCAG
ATTATATAGCATGGACCAGAGATGTGTTTGACAAGTAAGTTATTGTTTAGA
ATACAATACCATTTGTCGGTAACAAGATATTCTTAAGTCTAAAGGATTAGT
TACATTCTCTGTTAAGATCAGTTACATCCACAGTTGCAATTGTTTGGGTGA
ACCAAAGTTCAACCCAGATGGTTGGATACACAGGTGCTGGGGTGTCCACAA
AAGATGGGCTGCGAGAGAGCCATACATTTATCTATTTGTGATCATACTTAC
ACAAACAAAAGATGCATCTAGTGATGGAACATTAAAGCTGCAATCTTCAAA
GACTGGATTAGTTGTCAAGTGTGGTTAAGGTGTTTGATTATGGGAAGAGGT
TAAGGGTGCATTTACATTAATGGTCATGAGAAAGGGTTGTTGTTAGGTGCA
GATTTTGTAAGAGAAAGGTGATTTACAGTGTTTACTATCTCAACAGCATCA
ATATTGGAACAAGATTTCTTCATTTTGGGTTTCAAGTAATAATGGATACAA
GTATTCAAAGGTTACAGGGGGTGTTCAAAAAAGGGTTTGATGTTTTACATT
ATGGTGTAATTTGGGTTGTTGATGAGGTTTCTTCACCCTGGTTTGAAAAGA
TCTGTACCGCGTGGGTTGAGGATAGTTTTGTTCATTATCTATAAACAAAAA
CTTCTTCATACTTAGGAAACAGTATGGTAGATACCAGAGGATACATACAAT
GGTAATGGCAATTCACAGGGAAATCCATGGTTCTTGTGTTACAGGGTAGCA
GAATTTATATAGAGTATTAAGGAATGGATGGCAAGGGGTGTGACAGTTTCT
CAATTCATTGCCATTTTTCAAAAAGTTTGATCCAGGGACATTGGTAAAAAG
TATATGTGGGGATTCTGATTTCAACAATTTGGTCAAAACATTGCTTAGTGC
GACAGATTTTATTACGTACAATCCATGCACATAACAATGGTAGTTTGGCAG
AGGAATTTGATAGAACTACAGGACTCTCTACAGGTGGAGAGATTTAATTGG
TCACATGCAAGTTTAATTACAGC TTTAGCAAAGGTGGTGTCCTGTGCATA
A
Codon-optimized glucoamylase gene sequence (amyA
protein) from Rhizopus delemar
(SEQ ID NO: 53)
ATGCAGTTATTCAACTTACCACTTAAGGTATCTTTCTTTCTAGTCTTATCT
TACTTTTCATTGTTAGTATCAGCTGCCTCTATACCAAGTTCAGCATCCGTA
CAACTAGATTCATACAATTACGACGGTTCAACATTCTCAGGAAAGATATAC
GTGAAAAATATTGCTTACAGCAAAAAGGTTACTGTGATTTACGCAGATGGG
TCAGACAACTGGAATAACAATGGAAACACAATTGCTGCTTCCTATTCTGCC
CCTATTTCTGGATCTAACTACGAATACTGGACTTTTTCAGCGAGTATAAAC
GGAATTAAGGAATTCTATATCAAATATGAAGTCTCTGGTAAGACCTACTAC
GATAACAACAACTCCGCAAACTACCAAGTTAGCACATCAAAGCCAACCACA
ACAACTGCTACTGCGACAACTACAACCGCACCAAGCACTTCTACTACAACA
CCTCCTAGTTCATCTGAGCCAGCAACTTTCCCAACTGGTAATTCCACTATT
TCTTCTTGGATCAAAAAACAAGAGGGTATCTCAAGATTCGCCATGCTTAGA
AATATCAATCCTCCAGGCTCTGCAACAGGATTCATTGCAGCATCTTTATCA
ACTGCGGGGCCAGACTACTACTACGCCTGGACTAGAGATGCAGCTTTGACA
TCAAATGTGATTGTTTATGAATACAACACAACTTTGTCCGGTAACAAGACA
ATCTTGAACGTCTTGAAGGATTATGTGACATTCTCTGTCAAGACTCAATCT
ACATCAACAGTTTGTAACTGTCTCGGCGAACCAAAGTTCAACCCTGATGGT
AGTGGTTACACTGGTGCTTGGGGTAGACCACAAAACGATGGTCCAGCAGAG
AGAGCTACAACTTTCATCTTGTTTGCTGACTCTTACCTAACACAAACCAAG
GATGCAAGCTACGTTACTGGAACACTAAAGCCTGCAATCTTTAAAGACCTG
GACTATGTTGTAAACGTTTGGTCAAATGGCTGCTTCGATCTATGGGAGGAA
GTGAACGGTGTTCACTTCTACACATTAATGGTCATGAGAAAGGGACTCTTG
CTTGGTGCAGACTTTGCTAAGAGAAACGGTGATTCTACACGTGCCTCCACT
TACTCCTCCACAGCTTCAACCATTGCCAACAAAATCTCTTCTTTCTGGGTC
AGCTCAAATAACTGGATTCAAGTTTCTCAATCAGTTACTGGTGGTGTTTCT
AAAAAGGGCCTGGATGTGTCAACCTTGCTTGCTGCCAATTTGGGCAGTGTT
GATGACGGGTTCTTCACCCCAGGTTCTGAAAAGATCCTCGCCACCGCAGTT
GCCGTTGAAGATTCATTTGCTAGTTTATACCCAATCAACAAAAATCTACCA
TCATACCTTGGAAATTCAATCGGTAGATATCCAGAGGATACATACAACGGT
AATGGAAACTCTCAGGGTAACCCTTGGTTTCTTGCAGTTACAGGGTACGCT
GAACTGTACTACAGAGCGATTAAGGAATGGATTGGTAATGGCGGCGTAACT
GTTAGTTCTATTTCTCTACCTTTCTTCAAAAAGTTCGATAGTTCTGCAACA
TCTGGTAAAAAGTACACAGTCGGCACTTCCGATTTTAACAATTTAGCTCAG
AACATAGCACTGGCAGCTGATCGTTTCTTGAGTACAGTCCAATTGCATGCC
CATAACAACGGTAGTTTGGCTGAAGAGTTTGATAGAACCACCGGTTTATCA
ACCGGCGCCAGAGATTTAACATGGTCCCATGCGTCTTTGATAACTGCTTCT
TACGCCAAGGCTGGGGCACCAGCTGCCTGA
Glucoamylase protein sequence (amyA protein) from
Rhizopus delemar
(SEQ ID NO: 40)
MQLFNLPLKVSFFLVLSYFSLLVSAASIPSSASVQLDSYNYDGSTFSGKIY
VKNIAYSKKVTVIYADGSDNWNNNGNTIAASYSAPISGSNYEYWTFSASIN
GIKEFYIKYEVSGKTYYDNNNSANYQVSTSKPTTTTATATTTTAPSTSTTT
PPSSSEPATFPTGNSTISSWIKKQEGISRFAMLRNINPPGSATGFIAASLS
TAGPDYYYAWTRDAALTSNVIVYEYNTTLSGNKTILNVLKDYVTFSVKTQS
TSTVCNCLGEPKFNPDGSGYTGAWGRPQNDGPAERATTFILFADSYLTQTK
DASYVTGTLKPAIFKDLDYVVNVWSNGCFDLWEEVNGVHFYTLMVMRKGLL
LGADFAKRNGDSTRASTYSSTASTIANKISSFWVSSNNWIQVSQSVTGGVS
KKGLDVSTLLAANLGSVDDGFFTPGSEKILATAVAVEDSFASLYPINKNLP
SYLGNSIGRYPEDTYNGNGNSQGNPWFLAVTGYAELYYRAIKEWIGNGGVT
VSSISLPFFKKFDSSATSGKKYTVGTSDFNNLAQNIALAADRFLSTVQLHA
HNNGSLAEEFDRTTGLSTGARDLTWSHASLITASYAKAGAPAA
Codon-optimized glucoamylase gene sequence (amyA
protein) from Rhizopus microsporus
(SEQ ID NO: 54)
ATGAAACTTATGAATCCATCTATGAAGGCATACGTTTTCTTTATCTTAAGC
TACTTCTCTTTACTCGTTAGCTCAGCTGCGGTGCCAACCTCTGCCGCCGTA
CAAGTTGAGTCATACAATTATGACGGTACCACTTTTTCAGGTAGAATATTC
GTCAAAAACATTGCCTACTCAAAGGTCGTAACAGTTATCTACTCCGATGGA
TCAGATAACTGGAACAATAACAACAACAAAGTTTCTGCAGCTTACTCAGAA
GCAATTTCTGGGTCTAACTACGAATACTGGACATTCTCCGCAAAGTTATCC
GGAATTAAACAGTTTTATGTCAAATACGAAGTTTCTGGTTCAACATATTAC
GACAACAACGGTACCAAAAACTACCAAGTCCAAGCAACCTCAGCGACATCT
ACAACAGCTACTGCAACCACAACTACAGCTACTGGCACAACAACTACTTCT
ACAGGTCCAACTAGTACTGCATCCGTATCATTCCCTACCGGTAACTCAACA
ATTTCTTCCTGGATAAAAAATCAAGAGGAAATCAGCCGTTTTGCTATGTTG
AGAAATATCAATCCACCTGGGTCTGCCACAGGGTTCATAGCCGCATCTCTG
TCCACAGCCGGCCCAGATTACTATTACTCTTGGACTAGAGATTCAGCACTA
ACAGCTAATGTGATCGCTTACGAATACAACACAACATTCACTGGAAACACC
ACCCTTCTTAAGTACTTGAAAGATTACGTTACATTTTCTGTCAAAAGCCAA
TCTGTATCTACCGTTTGTAACTGTCTGGGAGAACCAAAGTTCAACGCTGAT
GGTAGTTCTTTTACAGGTCCATGGGGCAGACCACAAAACGACGGACCAGCA
GAGAGAGCTGTTACTTTTATGTTGATTGCTGACAGCTACTTGACTCAAACT
AAGGACGCATCCTACGTTACCGGTACATTAAAGCCAGCAATCTTCAAAGAT
CTTGATTACGTAGTTTCTGTTTGGTCTAACGGTTGCTACGATTTATGGGAA
GAGGTTAATGGTGTTCATTTCTATACTCTCATGGTCATGAGAAAGGGTTTG
ATCTTAGGTGCCGACTTCGCTGCTAGAAATGGTGACTCTAGTAGAGCTTCA
ACCTACAAGCAAACTGCATCAACAATGGAATCAAAGATCAGTTCTTTTTGG
TCAGATTCTAACAACTACGTCCAAGTTTCTCAATCAGTTACCGCCGGAGTG
TCAAAAAAGGGACTAGATGTTAGTACACTATTGGCGGCCAACATTGGTAGT
CTGCCTGATGGCTTTTTCACTCCAGGCTCCGAAAAGATATTGGCTACAGCA
GTGGCGTTAGAAAATGCATTCGCATCCTTGTACCCAATTAACTCTAACCTA
CCTTCTTACTTGGGTAACTCAATTGGAAGATATCCTGAGGATACATACAAC
GGTAATGGCAACTCTCAGGGGAATCCATGGTTCCTTGCCGTCAACGCATAC
GCAGAACTTTACTACAGAGCTATTAAGGAATGGATTAGTAATGGCAAGGTG
ACAGTATCCAATATCTCACTACCTTTCTTCAAAAAGTTTGATTCTTCCGCC
ACTTCTGGAAAGACATACACTGCTGGTACATCAGATTTCAATAACTTGGCT
CAGAACATTGCTTTAGGCGCCGATAGATTCCTGTCTACTGTTAAGTTCCAC
GCATACACTAACGGGAGTCTATCAGAAGAGTACGATAGATCTACCGGTATG
AGTACTGGGGCTCGTGATTTAACATGGTCCCATGCTTCATTGATCACAGTG
GCGTACGCAAAGGCCGGTAGTCCTGCAGCTTAG
Glucoamylas protein sequence (amyA protein) from
Rhizopus microsporus
(SEQ ID NO: 41)
MKLMNPSMKAYVFFILSYFSLLVSSAAVPTSAAVQVESYNYDGTTFSGRIF
VKNIAYSKVVTVIYSDGSDNWNNNNNKVSAAYSEAISGSNYEYWTFSAKLS
GIKQFYVKYEVSGSTYYDNNGTKNYQVQATSATSTTATATTTTATGTTTTS
TGPTSTASVSFPTGNSTISSWIKNQEEISRFAMLRNINPPGSATGFIAASL
STAGPDYYYSWTRDSALTANVIAYEYNTTFTGNTTLLKYLKDYVTFSVKSQ
SVSTVCNCLGEPKFNADGSSFTGPWGRPQNDGPAERAVTFMLIADSYLTQT
KDASYVTGTLKPAIFKDLDYVVSVWSNGCYDLWEEVNGVHFYTLMVMRKGL
ILGADFAARNGDSSRASTYKQTASTMESKISSFWSDSNNYVQVSQSVTAGV
SKKGLDVSTLLAANIGSLPDGFFTPGSEKILATAVALENAFASLYPINSNL
PSYLGNSIGRYPEDTYNGNGNSQGNPWFLAVNAYAELYYRAIKEWISNGKV
TVSNISLPFFKKFDSSATSGKTYTAGTSDFNNLAQNIALGADRFLSTVKFH
AYTNGSLSEEYDRSTGMSTGARDLTWSHASLITVAYAKAGSPAA

Trehalose-6-phosphate synthase gene and protein sequences are well known to one of ordinary skill in the art. Non-limiting examples of trehalose-6-phosphate synthase gene and protein sequences include:

TPS1 gene sequence from Saccharomyces cerevisiae
(SEQ ID NO: 55)
ATGACTACGGATAACGCTAAGGCGCAACTGACCTCGTCTTCAGGGGGTAAC
ATTATTGTGGTGTCCAACAGGCTTCCCGTGACAATCACTAAAAACAGCAGT
ACGGGACAGTACGAGTACGCAATGTCGTCCGGAGGGCTGGTCACGGCGTTG
GAAGGGTTGAAGAAGACGTACACTTTCAAGTGGTTCGGATGGCCTGGGCTA
GAGATTCCTGACGATGAGAAGGATCAGGTGAGGAAGGACTTGCTGGAAAAG
TTTAATGCCGTACCCATCTTCCTGAGCGATGAAATCGCAGACTTACACTAC
AACGGGTTCAGTAATTCTATTCTATGGCCGTTATTCCATTACCATCCTGGT
GAGATCAATTTCGACGAGAATGCGTGGTTGGCATACAACGAGGCAAACCAG
ACGTTCACCAACGAGATTGCTAAGACTATGAACCATAACGATTTAATCTGG
GTGCATGATTACCATTTGATGTTGGTTCCGGAAATGTTGAGAGTCAAGATT
CACGAGAAGCAACTGCAAAACGTTAAGGTCGGGTGGTTCCTGCACACACCA
TTCCCTTCGAGTGAAATTTACAGAATCTTACCTGTCAGACAAGAGATTTTG
AAGGGTGTTTTGAGTTGTGATTTAGTCGGGTTCCACACATACGATTATGCA
AGACATTTCTTGTCTTCCGTGCAAAGAGTGCTTAACGTGAACACATTGCCT
AATGGGGTGGAATACCAGGGCAGATTCGTTAACGTAGGGGCCTTCCCTATC
GGTATCGACGTGGACAAGTTCACCGATGGGTTGAAAAAGGAATCCGTACAA
AAGAGAATCCAACAATTGAAGGAAACTTTCAAGGGCTGCAAGATCATAGTT
GGTGTCGACAGGCTGGATTACATCAAAGGTGTGCCTCAGAAGTTGCACGCC
ATGGAAGTGTTTCTGAACGAGCATCCAGAATGGAGGGGCAAGGTTGTTCTG
GTACAGGTTGCAGTGCCAAGTCGTGGAGATGTGGAAGAGTACCAATATTTA
AGATCTGTGGTCAATGAGTTGGTCGGTAGAATCAACGGTCAGTTCGGTACT
GTGGAATTCGTCCCCATCCATTTCATGCACAAGTCTATACCATTTGAAGAG
CTGATTTCGTTATATGCTGTGAGCGATGTCTGTTTGGTCTCGTCCACCCGT
GATGGTATGAACTTGGTTTCCTACGAATATATTGCTTGCCAAGAAGAAAAG
AAAGGTTCCTTAATCCTGAGTGAGTTCACAGGTGCCGCACAATCCTTGAAT
GGTGCTATTATTGTAAATCCTTGGAACACCGATGATCTTTCTGATGCCATC
AACGAGGCCTTGACTTTGCCCGATGTAAAGAAAGAAGTTAACTGGGAAAAA
CTTTACAAATACATCTCTAAATACACTTCTGCCTTCTGGGGTGAAAATTTC
GTCCATGAATTATACAGTACATCATCAAGCTCAACAAGCTCCTCTGCCACC
AAAAACTGA
Tps1 protein sequence from Saccharomyces cerevisiae
(SEQ ID NO: 43):
MTTDNAKAQLTSSSGGNIIVVSNRLPVTITKNSSTGQYEYAMSSGGLVTAL
EGLKKTYTFKWFGWPGLEIPDDEKDQVRKDLLEKFNAVPIFLSDEIADLHY
NGFSNSILWPLFHYHPGEINFDENAWLAYNEANQTFTNEIAKTMNHNDLIW
VHDYHLMLVPEMLRVKIHEKQLQNVKVGWFLHTPFPSSEIYRILPVRQEIL
KGVLSCDLVGFHTYDYARHFLSSVQRVLNVNTLPNGVEYQGRFVNVGAFPI
GIDVDKFTDGLKKESVQKRIQQLKETFKGCKIIVGVDRLDYIKGVPQKLHA
MEVFLNEHPEWRGKVVLVQVAVPSRGDVEEYQYLRSVVNELVGRINGQFGT
VEFVPIHFMHKSIPFEELISLYAVSDVCLVSSTRDGMNLVSYEYIACQEEK
KGSLILSEFTGAAQSLNGAIIVNPWNTDDLSDAINEALTLPDVKKEVNWEK
LYKYISKYTSAFWGENFVHELYSTSSSSTSSSATKN

Trehalose-6-phosphate phosphatase gene and protein sequences are well known to one of ordinary skill in the art. Non-limiting examples of Trehalose-6-phosphate phosphatase gene and protein sequences include:

TPS2 gene sequence from Saccharomyces cerevisiae
(SEQ ID NO: 56)
ATGACCACCACTGCCCAAGACAATTCTCCAAAGAAGAGACAGCGTATCATC
AATTGTGTCACGCAGCTGCCCTACAAAATCCAATTGGGAGAAAGCAACGAT
GACTGGAAAATATCTGCTACTACAGGTAACAGCGCATTATATTCCTCTCTA
GAATACCTTCAATTTGATTCTACCGAGTACGAGCAACACGTTGTTGGTTGG
ACCGGCGAAATAACAAGAACCGAACGCAACCTGTTTACTAGAGAAGCGAAA
GAGAAACCACAGGATCTGGACGATGACCCACTATATTTAACAAAAGAGCAG
ATCAATGGGTTGACTACTACTCTACAAGATCATATGAAATCTGATAAAGAG
GCAAAGACCGATACTACTCAAACAGCTCCCGTTACCAATAACGTTCATCCC
GTTTGGCTACTTAGAAAAAACCAGAGTAGATGGAGAAATTACGCGGAAAAA
GTAATTTGGCCAACCTTCCACTACATCTTGAATCCTTCAAATGAAGGTGAG
CAAGAAAAAAACTGGTGGTACGACTACGTCAAGTTTAACGAAGCTTATGCA
CAAAAAATCGGGGAAGTTTACAGGAAGGGTGACATCATCTGGATCCATGAC
TACTACCTACTGCTATTGCCTCAACTACTGAGAATGAAATTTAACGACGAA
TCTATCATTATTGGTTATTTCCATCATGCCCCATGGCCTAGTAATGAATAT
TTTCGCTGTTTGCCACGTAGAAAACAAATCTTAGATGGTCTTGTTGGGGCC
AATAGAATTTGTTTCCAAAATGAATCTTTCTCCCGTCATTTTGTATCGAGT
TGTAAAAGATTACTCGACGCAACCGCCAAGAAATCTAAAAACTCTTCCGAT
AGTGATCAATATCAAGTGTCTGTGTACGGTGGTGACGTACTCGTAGATTCT
TTGCCTATAGGTGTTAACACAACTCAAATACTGAAAGATGCTTTCACGAAG
GATATAGATTCCAAGGTTCTTTCCATCAAGCAAGCTTATCAAAACAAAAAA
ATTATTATTGGTAGAGATCGTCTGGATTCCGTCAGAGGCGTCGTTCAAAAA
TTAAGAGCTTTTGAAACTTTCTTGGCCATGTATCCAGAATGGCGAGATCAA
GTGGTATTGATCCAGGTCAGCAGTCCTACTGCTAACAGAAATTCCCCCCAA
ACTATCAGATTGGAACAACAAGTCAACGAGTTGGTTAATTCCATAAATTCT
GAATATGGTAATTTGAATTTTTCTCCCGTCCAGCATTATTATATGAGAATC
CCTAAAGATGTATACTTGTCCTTACTAAGAGTTGCAGACTTATGTTTAATC
ACAAGTGTTAGAGACGGTATGAATACCACTGCTTTGGAATACGTCACTGTG
AAATCTCACATGTCGAACTTTTTATGCTACGGAAATCCATTGATTTTAAGT
GAGTTTTCTGGCTCTAGTAACGTATTGAAAGATGCCATTGTCGTTAACCCA
TGGGATTCGGTGGCCGTGGCTAAATCTATTAACATGGCTTTGAAATTGGAC
AAGGAAGAAAAGTCCAATTTAGAATCAAAATTATGGAAAGAAGTTCCTACA
ATTCAAGATTGGACTAATAAGTTTTTGAGTTCATTAAAGGAAAAGGCGTCA
TCTGATGATGATGTGGAAAGGAAAATGACTCCAGCACTTAATAGACCTGTT
CTTTTAGAAAACTACAAGCAGGCTAAGCGTAGATTATTCCTTTTTGATTAC
GATGGTACTTTGACCCCAATTGTCAAAGACCCAGCTGCAGCTATTCCATCG
GCAAGACTTTATACAATTCTACAAAAATTATGTGCCGATCCTCATAATCAA
ATCTGGATTATTTCTGGTCGTGACCAGAAGTTTTTGAACAAGTGGTTAGGC
GGTAAACTTCCTCAACTGGGTCTAAGTGCGGAGCATGGATGTTTCATGAAA
GATGTTTCTTGCCAAGATTGGGTCAATTTGACCGAAAAAGTTGATATGTCT
TGGCAAGTACGCGTCAATGAAGTGATGGAAGAATTTACCACAAGGACCCCA
GGTTCATTCATCGAAAGAAAGAAAGTCGCTCTAACTTGGCATTATAGACGT
ACCGTTCCAGAATTGGGTGAATTCCACGCCAAAGAACTGAAAGAAAAATTG
TTATCATTTACTGATGACTTCGATTTAGAGGTCATGGATGGTAAAGCAAAC
ATTGAAGTTCGTCCAAGATTCGTCAACAAAGGTGAAATAGTCAAGAGACTA
GTCTGGCATCAACATGGCAAACCACAGGACATGTTGAAGGGAATCAGTGAA
AAACTACCTAAGGATGAAATGCCTGATTTTGTATTATGTCTGGGTGATGAC
TTCACTGACGAAGACATGTTTAGACAGTTGAATACCATTGAAACTTGTTGG
AAAGAAAAATATCCTGACCAAAAAAATCAATGGGGCAACTACGGATTCTAT
CCTGTCACTGTGGGATCTGCATCCAAGAAAACTGTCGCAAAGGCTCATTTA
ACCGATCCTCAGCAAGTCCTGGAGACTTTAGGTTTACTTGTTGGTGATGTC
TCTCTCTTCCAAAGTGCTGGTACGGTCGACCTGGATTCCAGAGGTCATGTC
AAGAATAGTGAGAGCAGTTTGAAATCAAAGCTAGCATCTAAAGCTTATGTT
ATGAAAAGATCGGCTTCTTACACCGGCGCAAAGGTTTGA
Tps2 protein sequence from Saccharomyces cerevisiae
(SEQ ID NO: 44):
MTTTAQDNSPKKRQRIINCVTQLPYKIQLGESNDDWKISATTGNSALFSSL
EYLQFDSTEYEQHVVGWTGEITRTERNLFTREAKEKPQDLDDDPLYLTKEQ
INGLTTTLQDHMKSDKEAKTDTTQTAPVTNNVHPVWLLRKNQSRWRNYAEK
VIWPTFHYILNPSNEGEQEKNWWYDYVKFNEAYAQKIGEVYRKGDIIWIHD
YYLLLLPQLLRMKFNDESIIIGYFHHAPWPSNEYFRCLPRRKQILDGLVGA
NRICFQNESFSRHFVSSCKRLLDATAKKSKNSSNSDQYQVSVYGGDVLVDS
LPIGVNTTQILKDAFTKDIDSKVLSIKQAYQNKKIIIGRDRLDSVRGVVQK
LRAFETFLAMYPEWRDQVVLIQVSSPTANRNSPQTIRLEQQVNELVNSINS
EYGNLNFSPVQHYYMRIPKDVYLSLLRVADLCLITSVRDGMNTTALEYVTV
KSHMSNFLCYGNPLILSEFSGSSNVLKDAIVVNPWDSVAVAKSINMALKLD
KEEKSNLESKLWKEVPTIQDWTNKFLSSLKEQASSNDDMERKMTPALNRPV
LLENYKQAKRRLFLFDYDGTLTPIVKDPAAAIPSARLYTILQKLCADPHNQ
IWIISGRDQKFLNKWLGGKLPQLGLSAEHGCFMKDVSCQDWVNLTEKVDMS
WQVRVNEVMEEFTTRTPGSFIERKKVALTWHYRRTVPELGEFHAKELKEKL
LSFTDDFDLEVMDGKANIEVRPRFVNKGEIVKRLVWHQHGKPQDMLKGISE
KLPKDEMPDFVLCLGDDFTDEDMFRQLNTIETCWKEKYPDQKNQWGNYGFY
PVTVGSASKKTVAKAHLTDPQQVLETLGLLVGDVSLFQSAGTVDLDSRGHV
KNSESSLKSKLASKAYVMKRSASYTGAKV

The function and advantage of these and other embodiments will be more fully understood from the examples below. The following examples are intended to illustrate the benefits of the present invention, but do not exemplify the full scope of the invention. Accordingly, it will be understood that the Examples section is not meant to limit the scope of the invention.

### EXAMPLES

### Example 1: Generation of Amylolytic Saccharomyces cerevisiae Strains

Described below are genetically modified S. cerevisiae yeast strains. The strains described include strains having genetic modifications that improve the lactate-consuming ability of ethanol producing yeasts.

Strain 1-3: Ura3Δ Saccharomyces cerevisiae Base Strain

Strain 1 (Ethanol Red®) is transformed with SEQ ID NO: 1. SEQ ID NO: 1 contains the following elements: i) an expression cassette for a mutant version of a 3-deoxy-D-arabino-heptulosonate-7-phosphate (DAHP) synthase gene from Saccharomyces cerevisiae (ARO4-OFP); and ii) flanking DNA for targeted chromosomal integration into the URA3 locus. Transformants were selected on synthetic complete media containing 3.5 g/L of p-fluorophenylalanine, and 1 g/L L-tyrosine (ScD-PFP). Resulting transformants were struck for single colony isolation on ScD-PFP. A single colony is selected. Correct integration of SEQ ID NO: 1 into one allele of locus A is verified by PCR in the single colony. A PCR verified isolate is designated Strain 1-1.

Stain 1-1 is transformed with SEQ ID NO: 2. SEQ ID NO: 2 contains the following elements: i) an expression cassette for an acetamidase (amdS) gene from Aspergillus nidulans; and ii) flanking DNA for targeted chromosomal integration into the URA3 locus. Transformants were selected on Yeast Nitrogen Base (without ammonium sulfate or amino acids) containing 80 mg/L uracil and 1 g/L acetamide as the sole nitrogen source. Resulting transformants were struck for single colony isolation on Yeast Nitrogen Base (without ammonium sulfate or amino acids) containing 80 mg/L uracil and 1 g/L acetamide as the sole nitrogen source. A single colony is selected. Correct integration of SEQ ID NO: 2 into the second allele of locus A is verified by PCR in the single colony. A PCR verified isolate is designated Strain 1-2.

Strain 1-2 is co-transformed with SEQ ID NO: 3 and SEQ ID NO: 4. SEQ ID NO:3 contains the following elements: i) an open reading frame for a cre recombinase from P1 bacteriophage, and ii) flanking DNA homologous to SEQ ID NO:4. SEQ ID NO: 4 contains the following elements: i) a 2μ origin of replication; ii) a URA3 selectable marker from Saccharomyces cerevisiae; and iii) flanking DNA containing a PGK promoter and CYC1 terminator from Saccharomyces cerevisiae. Transformants were selected on synthetic dropout media lacking uracil (ScD-Ura). Resulting transformants were struck for single colony isolation on ScD-Ura. A single colony is selected. The isolated colony is screened for growth on ScD-PFP and Yeast Nitrogen Base (without ammonium sulfate or amino acids) containing 80 mg/L uracil and 1 g/L acetamide as the sole nitrogen source. Loss of the ARO4-OFP and amdS genes is verified by PCR. The PCR verified isolate is struck to YNB containing 5-FOA to select for loss of the 2μ plasmid. The PCR verified isolate is designated Strain 1-3.

Strain 1-4: Saccharomyces cerevisiae Expressing Two Codon Optimized Variants of the Saccharomycopsis fibuligera Glucoamylase at the First Allele of CYB2

Strain 1-3 is co-transformed with SEQ ID NO: 5 and SEQ ID NO: 6. SEQ ID NO:5 contains the following elements: i) DNA homologous to the 5′ region of the native CYB2 gene; and ii) an expression cassette for a unique codon optimized variant of the Saccharomycopsis fibuligera glucoamylase (SEQ ID NO: 38), under control of the TDH3 promoter and CYC1 terminator; and iii) the URA3 promoter as well as a portion of the URA3 gene. SEQ ID NO: 6 contains the following elements: i) a portion of the URA3 gene and terminator; and ii) an expression cassette for a unique codon optimized variant of the Saccharomycopsis fibuligera glucoamylase, under control of the PGK promoter and RPL3 terminator; and iii) DNA homologous to the 3′ region of the native CYB2 gene. Transformants were selected on ScD-Ura. Resulting transformants were struck for single colony isolation on ScD-Ura. A single colony is selected. Correct integration of SEQ ID NO: 5 and SEQ ID NO: 6 at one allele of CYB2 is verified by PCR. The PCR verified isolate is designated Strain 1-4.

Strain 1-5: Saccharomyces cerevisiae Expressing Four Codon Optimized Variants of the Saccharomycopsis fibuligera Glucoamylase at the Second Allele of CYB2

Strain 1-4 is co-transformed with SEQ ID NO: 7 and SEQ ID NO: 8. SEQ ID NO: 7 contains the following elements: i) DNA homologous to the 5′ region of the native CYB2 gene; and ii) an expression cassette for a unique codon optimized variant of the Saccharomycopsis fibuligera glucoamylase, under control of the TDH3 promoter and CYC1 terminator; and iii) the TEF1 promoter and a portion of the Aspergillus nidulans acetamidase gene (amdS). SEQ ID NO: 8 contains the following elements: i) a portion of the Aspergillus nidulans acetamidase gene (amdS) and ADH1 terminator; and ii) an expression cassette for a unique codon optimized variant of the Saccharomycopsis fibuligera glucoamylase, under control of the PGK promoter and RPL3 terminator; and iii) DNA homologous to the 3′ region of the native CYB2 gene. Transformants were selected on Yeast Nitrogen Base (without ammonium sulfate or amino acids) containing 80 mg/L uracil and 1 g/L acetamide as the sole nitrogen source. Resulting transformants were struck for single colony isolation on Yeast Nitrogen Base (without ammonium sulfate or amino acids) containing 80 mg/L uracil and 1 g/L acetamide as the sole nitrogen source. A single colony is selected. Correct integration of SEQ ID NO: 7 and SEQ ID NO: 8 at the remaining allele of CYB2 is verified by PCR. The PCR verified isolate is designated Strain 1-5.

Strain 1-6: Recycling the URA3 and amdS Markers Via Cre Recombinase in Strain 1-5

Strain 1-5 is transformed with SEQ ID NO: 9. SEQ ID NO: 9 contains the following elements: i) an expression cassette for a mutant version of a 3-deoxy-D-arabino-heptulosonate-7-phosphate (DAHP) synthase gene from Saccharomyces cerevisiae (ARO4-OFP); 2) an expression cassette for a cre recombinase from P1 bacteriophage; 3) an expression cassette containing the native URA3, and 4) the Saccharomyces cerevisiae CEN6 centromere. Transformants were selected on synthetic complete media containing 3.5 g/L of p-fluorophenylalanine, and 1 g/L L-tyrosine (ScD-PFP). Resulting transformants were struck for single colony isolation on ScD-PFP. A single colony is selected. The PCR verified isolate is designated Strain 1-6.

### Strain 1-7: Restoring the Native URA3 at the Original Locus in Strain 1-6

Strain 1-6 is transformed with SEQ ID NO: 10. SEQ ID NO: 10 contains the follow elements: 1) an expression cassette for the native URA3, with 5′ and 3′ homology to the disrupted URA3 locus in Strain 1-6. Transformants were selected on ScD-ura. Resulting transformants were struck for single colony isolate on ScD-ura. A single colony is selected. The PCR verified isolate is designated Strain 1-7.

Strain 1-8: Saccharomyces cerevisiae Expressing a Modified Rhizopus oryzae Glucoamylase at the First Allele of CYB2.

Strain 1-3 is co-transformed with SEQ ID NO: 11 and SEQ ID NO: 12. SEQ ID NO: 11 and SEQ ID NO: 12 are similar to SEQ ID NO: 5 and SEQ ID NO: 6 with the following difference: the Saccharomycopsis fibuligera glucoamylase is replaced with the Rhizopus oryzae glucoamylase (SEQ ID NO: 39). Transformants are selected on ScD-Ura. Resulting transformants were struck for single colony isolation on ScD-Ura. Single colonies were selected, and the correct integration of the expression cassette is confirmed by PCR. Three independent transformants were tested in a shake flask fermentation and a representative isolate is designated Strain 1-8.

Strain 1-9: Saccharomyces cerevisiae Expressing a Modified Rhizopus oryzae Glucoamylase at the Second Allele of CYB2.

Strain 1-8 is co-transformed with SEQ ID NO: 13 and SEQ ID NO: 14. SEQ ID NO: 13 and SEQ ID NO: 14 are similar to SEQ ID NO: 7 and SEQ ID NO: 8 with the following difference: the Saccharomycopsis fibuligera glucoamylase is replaced with the Rhizopus oryzae glucoamylase. Transformants were selected on YNB+acetamide plates. Resulting transformants were struck for single colony isolation on YNB+acetamide plates. Single colonies were selected, and the correct integration of the expression cassette is confirmed by PCR. Three independent transformants were tested in a shake flask fermentation and a representative isolate is designated Strain 1-9.

Strain 1-10: Recycling the URA3 and amdS Markers Via Cre Recombinase in Strain 1-9

Strain 1-9 is transformed with SEQ ID NO: 9. Transformants were selected on synthetic complete media containing 3.5 g/L of p-fluorophenylalanine, and 1 g/L L-tyrosine (ScD-PFP). Resulting transformants were struck for single colony isolation on ScD-PFP. A single colony is selected. The PCR verified isolate is designated Strain 1-10.

### Strain 1-11: Restoring the Native URA3 at the Original Locus in Strain 1-10

Strain 1-10 is transformed with SEQ ID NO: 10. Transformants were selected on ScD-ura. Resulting transformants were struck for single colony isolate on ScD-ura. A single colony is selected. The PCR verified isolate is designated Strain 1-11.

Strain 1-12: Saccharomyces cerevisiae Expressing a Modified Rhizopus delemar Glucoamylase at the First Allele of FCY1.

Strain 1-3 is co-transformed with SEQ ID NO: 15 and SEQ ID NO: 16. SEQ ID NO: 15 contains the following elements: i) DNA homologous to the 5′ region of the native FCY1 gene; and ii) an expression cassette for a unique codon optimized variant of the Rhizopus delemar glucoamylase (SEQ ID NO: 40), under control of the TDH3 promoter and CYC1 terminator; and iii) the URA3 promoter as well as a portion of the URA3 gene. SEQ ID NO: 16 contains the following elements: i) a portion of the URA3 gene and terminator; and ii) an expression cassette for a unique codon optimized variant of the Rhizopus delemar glucoamylase, under control of the PGK promoter and GAL10 terminator; and iii) DNA homologous to the 3′ region of the native FCY1 gene. Transformants were selected on ScD-Ura. Resulting transformants were struck for single colony isolation on ScD-Ura. Single colonies were selected, and the correct integration of the expression cassette is confirmed by PCR. Three independent transformants were tested in a shake flask fermentation and a representative isolate is designated Strain 1-12.

Strain 1-13: Saccharomyces cerevisiae Expressing a Modified Rhizopus delemar Glucoamylase at the Second Allele of FCY1.

Strain 1-12 is co-transformed with SEQ ID NO: 17 and SEQ ID NO: 18. SEQ ID NO: 17 contains the following elements: i) DNA homologous to the 5′ region of the native FCY1 gene; and ii) an expression cassette for a unique codon optimized variant of the Rhizopus delemar glucoamylase, under control of the TDH3 promoter and CYC1 terminator; and iii) the TEF1 promoter as well as a portion of the Aspergillus nidulans amdS gene. SEQ ID NO: 18 contains the following elements: i) a portion of the Aspergillus nidulans acetamidase (amdS) gene and ADH1 terminator; and ii) an expression cassette for a unique codon optimized variant of the Rhizopus delemar glucoamylase, under control of the PGK promoter and GAL10 terminator; and iii) DNA homologous to the 3′ region of the native FCY1 gene. Transformants were selected on YNB+acetamide plates. Resulting transformants were struck for single colony isolation on YNB+acetamide plates. Single colonies were selected, and the correct integration of the expression cassette is confirmed by PCR. Three independent transformants were tested in a shake flask fermentation and a representative isolate is designated Strain 1-13.

Strain 1-14: Recycling the URA3 and amdS Markers Via Cre Recombinase in Strain 1-13

Strain 1-13 is transformed with SEQ ID NO: 9. Transformants were selected on synthetic complete media containing 3.5 g/L of p-fluorophenylalanine, and 1 g/L L-tyrosine (ScD-PFP). Resulting transformants were struck for single colony isolation on ScD-PFP. A single colony is selected. The PCR verified isolate is designated Strain 1-14.

### Strain 1-15: Restoring the Native URA3 at the Original Locus in Strain 1-14

Strain 1-14 is transformed with SEQ ID NO: 10. Transformants were selected on ScD-ura. Resulting transformants were struck for single colony isolate on ScD-ura. A single colony is selected. The PCR verified isolate is designated Strain 1-15.

Strain 1-16: Saccharomyces cerevisiae Expressing a Modified Rhizopus microsporus Glucoamylase at the First Allele of FCY1.

Strain 1-3 is co-transformed with SEQ ID NO: 19 and SEQ ID NO: 20. SEQ ID NO: 19 is similar to SEQ ID NO: 15 with the following difference: the Rhizopus delemar glucoamylase is replaced with the Rhizopus microsporus glucoamylase (SEQ ID NO: 41). SEQ ID NO: 20 contains the following elements: i) a portion of the URA3 gene and terminator; and ii) DNA homologous to the 3′ region of the native FCY1 gene. Transformants were selected on ScD-Ura. Resulting transformants were struck for single colony isolation on ScD-Ura. Single colonies were selected, and the correct integration of the expression cassette is confirmed by PCR. Three independent transformants were tested in a shake flask fermentation and a representative isolate is designated Strain 1-16.

Strain 1-17: Saccharomyces cerevisiae Expressing a Modified Rhizopus microsporus Glucoamylase at the Second Allele of FCY1.

Strain 1-16 is co-transformed with SEQ ID NO: 21 and SEQ ID NO: 22. SEQ ID NO: 21 is similar to SEQ ID NO: 17 with the following difference: the Rhizopus delemar glucoamylase is replaced with the Rhizopus microsporus glucoamylase. SEQ ID NO: 22 contains the following elements: i) a portion of the Aspergillus nidulans acetamidase (amdS) gene and TEF1 terminator; and ii) DNA homologous to the 3′ region of the native FCY1 gene. Transformants were selected on YNB+acetamide plates. Resulting transformants were struck for single colony isolation on YNB+acetamide plates. Single colonies were selected, and the correct integration of the expression cassette is confirmed by PCR. Three independent transformants were tested in a shake flask fermentation and a representative isolate is designated Strain 1-17.

Strain 1-18: Recycling the URA3 and amdS Markers Via Cre Recombinase in Strain 1-17

Strain 1-17 is transformed with SEQ ID NO: 9. Transformants were selected on synthetic complete media containing 3.5 g/L of p-fluorophenylalanine, and 1 g/L L-tyrosine (ScD-PFP). Resulting transformants were struck for single colony isolation on ScD-PFP. A single colony is selected. The PCR verified isolate is designated Strain 1-18.

### Strain 1-19: Restoring the Native URA3 at the Original Locus in Strain 1-18

Strain 1-18 is transformed with SEQ ID NO: 10. Transformants were selected on ScD-ura. Resulting transformants were struck for single colony isolate on ScD-ura. A single colony is selected. The PCR verified isolate is designated Strain 1-19.

Strain 1-20: Saccharomyces cerevisiae Expressing a Modified Rhizopus oryzae Glucoamylase at Both Alleles of CYB2, and a Bacillus cereus Glyceraldehyde-3-Phosphate Dehydrogenase at Both Alleles of GDP1.

Strain 1-10 is co-transformed with SEQ ID NO: 23 and SEQ ID NO: 24, and SEQ ID NO: 25 and SEQ ID NO: 26.

SEQ ID NO: 23 contains the following elements: i) DNA homologous to the 5′ region of the native GPD1 gene; and ii) an expression cassette for a unique codon optimized variant of the Bacillus cereus glyceraldehyde-3-phosphate dehydrogenase (SEQ ID NO: 42), under control of the PGK1 promoter and CYC1 terminator; and iii) loxP recombination site, and iv) a portion of the URA3 gene. SEQ ID NO: 24 contains the following elements: i) a portion of the URA3 gene and URA3 terminator; and ii) loxP recombination site; and iii) DNA homologous to the 3′ region of the native GPD1 gene.

SEQ ID NO: 25 contains the following elements: i) DNA homologous to the 5′ region of the native GPD1 gene; and ii) an expression cassette for a unique codon optimized variant of the Bacillus cereus glyceraldehyde-3-phosphate dehydrogenase, under control of the PGK1 promoter and CYC1 terminator; and iii) loxP recombination sites, and iv) the TEF1 promoter and a portion of the Aspergillus nidulans acetamidase (amdS) gene. SEQ ID NO: 26 contains the following elements: i) a portion of the amdS gene and TEF1 terminator; and ii) loxP recombination site, and iii) DNA homologous to the 3′ region of the native GPD1 gene.

Transformants were selected on YNB+acetamide plates. Resulting transformants were struck for single colony isolation on YNB+acetamide plates. Single colonies were selected, and the correct integration of the expression cassette is confirmed by sequencing. Three independent transformants were tested in a shake flask fermentation and a representative isolate is designated Strain 1-20.

Strain 1-21: Saccharomyces cerevisiae Expressing a Modified Rhizopus oryzae Glucoamylase at Both Alleles of CYB2, and a Deletion of Both Alleles of GPP1

Strain 1-10 is transformed with SEQ ID NO: 27. SEQ ID NO: 27 contains the following elements: i) DNA homologous to the 5′ region of the native GPP1 gene; and ii) from Kluyveromyces lactis, the URA3 promoter as well as the URA3 gene and URA3 terminator; and iv) loxP recombination sites flanking the URA3 cassette; and iv) DNA homologous to the 3′ region of the native GPP1 gene.

Transformants were selected on ScD-Ura. Resulting transformants were struck for single colony isolation on ScD-Ura. Single colonies were selected, and the correct integration of the expression cassette is confirmed by sequencing. Three independent transformants were tested in a shake flask fermentation and a representative isolate is designated Strain 1-21.

Strain 1-22: Saccharomyces cerevisiae Expressing a Modified Rhizopus oryzae Glucoamylase at Both Alleles of CYB2, and a Bacillus cereus Glyceraldehyde-3-Phosphate Dehydrogenase at Both Alleles of GPP1.

Strain 1-10 is co-transformed with SEQ ID NO: 28 and SEQ ID NO: 29, and SEQ ID NO: 30 and SEQ ID NO: 31.

SEQ ID NO: 28 and SEQ ID NO: 29 are similar to SEQ ID NO: 23 and SEQ ID NO: 24 with the following difference: the DNA homologous to the native GPD1 gene in SEQ ID NO: 23 and SEQ ID NO: 24 is replaced with the DNA homologous to the native GPP1 gene. SEQ ID NO: 30 and SEQ ID NO: 31 are similar to SEQ ID NO: 25 and SEQ ID NO: 26 with the following difference: the DNA homologous to the native GPD1 gene in SEQ ID NO: 25 and SEQ ID NO: 26 is replaced with the DNA homologous to the native GPP1 gene.

The plasmid sequence for the GAPN integration cassette is:

(SEQ ID NO: 59)
TGAGCTCCGGGTGGGAGGAAGGCGCGGCAATTAGAATGTGTGGGTGCGGAA
GCTCGCCGCTCCCATCAAGAGAGTGGAAGACGTATGGTCTGGGTGCGAAGT
ACCACCACGTTTCTTTTTCATCTCTTAAGTGGGATTCTTACGAAACACGTC
ACAGGGTCAAAAGAAAGAGAACAAAAGCAATATTGTAATTGTCTCAGTCCA
CGGCAATGACATGGCATGGCCCCGAAGGCTTTTTTTGTCTGTCTTCCTTGG
GTCTTACCCCGCCACGCGTTAATAGTGAGACAAGCAGGAAATCCGTATCAT
TTTCTCGCATACACGAACCCGCGTGCGCCTGGTAAATTGCAGGATTCTCAT
TGTCCGGTTTTCTTTATGGGAATAATCATCATCACCATTATCACTGTTACT
CTTGCGATCATCATCATTAACATAATTTTTTTAACGCTGTTTGATGATGGT
ATGTGCTTTTATTGTTCCTTACTCACCTTTTCCTTTGTGTCTTTTAATTTT
GACCATTTTGACCATTTTGACCTTTGATGATGTGTGAGTTCCTCTTTTCTT
TTTTTCTTTTCTTTTTTCCTTTTTTTTTCTTTTCTTACTGTGTTAATCACT
TTCTTTCCTTTTTGTTCATATTGTCGTCTTGTTCATTTTCGTTCAATTGAT
AATGTATATAAATCTTTCGTAAGTATCTCTTGATTGCCATTTTTTTCTTTC
CAAGTTTCCTTGTTCTCGAGGCCAGAAAAAGGAAGTGTTTCCCTCCTTCTT
GAATTGATGTTACCCTCATAAAGCACGTGGCCTCTTATCGAGAAAGAAATT
ACCGTCGCTCGTGATTTGTTTGCAAAAAGAACAAAACTGAAAAAACCCAGA
CACGCTCGACTTCCTGTCTTCCTATTGATTGCAGCTTCCAATTTCGTCACA
CAACAAGGTCCTAGCGACGGCTCACAGGTTTTGTAACAAGCAATCGAAGGT
TCTGGAATGGCGGGAAAGGGTTTAGTACCACATGCTATGATGCCCACTGTG
ATCTCCAGAGCAAAGTTCGTTCGATCGTACTGTTACTCTCTCTCTTTCAAA
CAGAATTGTCCGAATCGTGTGACAACAACAGCCTGTTCTCACACACTCTTT
TCTTCTAACCAAGGGGGTGGTTTAGTTTAGTAGAACCTCGTGAAACTTACA
TTTACATATATATAAACTTGCATAAATTGGTCAATGCAAGAAATACATATT
TGGTCTTTTCTAATTCGTAGTTTTTCAAGTTCTTAGATGCTTTCTTTTTCT
CTTTTTTACAGATCATCAAGGAAGTAATTATCTACTTTTTACAAGTCTAGA
ATGACAACATCAAATACCTACAAATTCTATCTAAACGGTGAATGGAGAGAA
TCTTCCTCTGGAGAAACTATTGAGATACCATCACCATACTTACATGAAGTG
ATCGGACAGGTTCAAGCAATCACTAGAGGAGAGGTTGACGAAGCGATTGCT
AGCGCTAAGGAAGCACAGAAATCTTGGGCTGAGGCATCTCTACAAGATAGA
GCTAAGTACTTGTACAAATGGGCAGATGAATTGGTAAACATGCAAGACGAA
ATCGCCGATATCATCATGAAGGAAGTGGGCAAGGGTTACAAAGACGCTAAA
AAGGAGGTTGTTAGAACCGCCGATTTCATCAGATACACCATTGAAGAGGCA
CTCCATATGCACGGTGAATCCATGATGGGCGATTCATTTCCTGGTGGAACA
AAATCTAAGCTAGCAATAATCCAAAGAGCGCCTCTGGGTGTAGTCTTAGCC
ATCGCTCCATTCAATTACCCTGTAAACCTTTCTGCTGCAAAATTGGCACCA
GCCTTAATTATGGGTAACGCTGTGATATTCAAGCCAGCAACTCAGGGTGCT
ATTTCCGGCATCAAAATGGTTGAAGCTTTGCATAAGGCTGGTTTGCCAAAG
GGTTTGGTTAACGTTGCCACAGGTAGAGGTAGCGTCATAGGCGATTATTTG
GTCGAACACGAAGGGATAAACATGGTTTCCTTCACCGGTGGCACTAACACT
GGTAAGCATTTAGCAAAAAAGGCCTCAATGATTCCATTAGTCTTGGAACTT
GGTGGCAAAGATCCAGGCATCGTTCGTGAAGATGCAGACCTACAAGATGCT
GCGAATCATATCGTATCTGGTGCGTTCAGTTACTCAGGGCAGAGATGTACA
GCCATTAAGAGAGTCCTTGTTCATGAAAATGTTGCTGATGAACTGGTATCA
TTGGTTAAGGAACAAGTGGCAAAGCTTTCTGTGGGATCACCAGAGCAAGAT
TCAACAATTGTTCCTCTGATTGACGATAAGTCCGCTGATTTTGTTCAGGGT
TTAGTGGACGATGCAGTCGAAAAGGGCGCTACAATTGTCATTGGGAACAAG
AGAGAACGTAACCTAATCTACCCAACATTGATTGATCACGTCACAGAGGAA
ATGAAAGTTGCCTGGGAGGAACCATTCGGTCCTATTCTTCCAATTATTAGA
GTTAGTAGCGACGAGCAAGCTATTGAAATTGCAAATAAGAGTGAGTTCGGA
TTACAAGCTTCTGTGTTTACCAAAGACATAAACAAGGCATTCGCAATCGCA
AATAAGATTGAGACTGGTTCAGTGCAAATCAACGGTAGAACAGAGAGAGGA
CCAGATCACTTTCCTTTTATCGGGGTTAAGGGATCTGGGATGGGTGCCCAA
GGCATCAGAAAGTCTTTGGAATCTATGACTAGAGAAAAAGTTACTGTCTTA
AATCTCGTATGATTAAACAGGCCCCTTTTCCTTTGTCGATATCATGTAATT
AGTTATGTCACGCTTACATTCACGCCCTCCTCCCACATCCGCTCTAACCGA
AAAGGAAGGAGTTAGACAACCTGAAGTCTAGGTCCCTATTTATTTTTTTAT
AGTTATGTTAGTATTAAGAACGTTATTTATATTTCAAATTTTTCTTTTTTT
TCTGTACAAACGCGTGTACGCATGTAACGGGCAGACG.

In SEQ ID NO: 59, the region encoded by nucleotides 1-729 is a GPP1 up flank region; the region encoded by nucleotides 730-1326 is a PGK promoter; the region encoded by nucleotides 1327-2766 is a codon optimized coding sequence for B. cereus GAPN; and the region encoded by nucleotides 2767-2995 is a terminator region.

Transformants were selected on YNB+acetamide plates. Resulting transformants were struck for single colony isolation on YNB+acetamide plates. Single colonies were selected, and the correct integration of the expression cassette is confirmed by sequencing. Three independent transformants were tested in a shake flask fermentation and a representative isolate is designated Strain 1-22.

Strain 1-23: Saccharomyces cerevisiae Expressing a Modified Saccharomycopsis fibuligera Glucoamlase at Both Alleles of CYB2, and a Bacillus cereus Glyceraldehyde-3-Phosphate Dehydrogenase at Both Alleles of GPP1.

Strain 1-6 is co-transformed with SEQ ID NO: 28 and SEQ ID NO: 29, and transformants are selected on ScD-Ura. Resulting transformants were struck for single colony isolation on ScD-Ura. Single colonies were selected, and the correct integration of the expression cassette is confirmed by PCR. Three independent transformants were moved forward for integration of the second copy of the expression cassette at the GPP1 locus.

Three independent sisters strains containing 1 copy of SEQ ID NO: 28 and SEQ ID NO: 29 were co-transformed with SEQ ID NO: 30 and SEQ ID NO: 31, and transformants were selected on YNB+acetamide plates. Resulting transformants were struck for single colony isolation on YNB+acetamide plates. Single colonies were selected, and the correct integration of the expression cassette is confirmed by PCR. Three independent transformants were tested in the fermentation condition described in TEST #5, and a representative isolate that demonstrated early fermentation rate and equivalent or higher final ethanol titer when compared to Strain 1 is designated Strain 1-23.

Strain 1-24: Saccharomyces cerevisiae Expressing a Modified Rhizopus delemar Glucoamylase at Both Alleles of FCY1, and a Bacillus cereus Glyceraldehyde-3-Phosphate Dehydrogenase at Both Alleles of GPP1.

Strain 1-14 is co-transformed with SEQ ID NO: 28 and SEQ ID NO: 29, and SEQ ID NO: 30 and SEQ ID NO: 31. Transformants were selected on YNB+acetamide plates. Resulting transformants were struck for single colony isolation on YNB+acetamide plates. Single colonies were selected, and the correct integration of the expression cassette is confirmed by sequencing. Three independent transformants were tested in a shake flask fermentation and a representative isolate is designated Strain 1-24.

Strain 1-25: Saccharomyces cerevisiae Expressing a Modified Rhizopus microsporus Glucoamylase at Both Alleles of FCY1, and a Bacillus cereus Glyceraldehyde-3-Phosphate Dehydrogenase at Both Alleles of GPP1.

Strain 1-18 is co-transformed with SEQ ID NO: 28 and SEQ ID NO: 29, and SEQ ID NO: 30 and SEQ ID NO: 31. Transformants were selected on YNB+acetamide plates. Resulting transformants were struck for single colony isolation on YNB+acetamide plates. Single colonies were selected, and the correct integration of the expression cassette is confirmed by sequencing. Three independent transformants were tested in a shake flask fermentation and a representative isolate is designated Strain 1-25.

Strain 1-26: Saccharomyces cerevisiae Expressing a Modified Rhizopus oryzae Glucoamylase at Both Alleles of CYB2, and a Bacillus cereus Glyceraldehyde-3-Phosphate Dehydrogenase at Both Alleles of DLD1.

Strain 1-10 is co-transformed with SEQ ID NO: 32 and SEQ ID NO: 33. SEQ ID NO: 32 and SEQ ID NO: 33 are similar to SEQ ID NO: 23 and SEQ ID NO: 24 with the following difference: the DNA homologous to the native GPD1 gene in SEQ ID NO: 23 and SEQ ID NO: 24 is replaced with the DNA homologous to the native DLD1 gene. Transformants were selected on ScD-Ura. Resulting transformants were struck for single colony isolation on ScD-Ura. Single colonies were selected, and the correct integration of the expression cassette is confirmed by PCR. Three independent transformants were moved forward for integration of the second copy of the expression cassette at the DLD1 locus.

Three independent sisters strains containing 1 copy of SEQ ID NO: 32 and SEQ ID NO: 33 were co-transformed with SEQ ID NO: 34 and SEQ ID NO: 35. SEQ ID NO: 34 and SEQ ID NO: 35 are similar to SEQ ID NO: 25 and SEQ ID NO: 26 with the following difference: the DNA homologous to the native GPD1 gene in SEQ ID NO: 25 and SEQ ID NO: 26 is replaced with the DNA homologous to the native DLD1 gene. Transformants were selected on YNB+acetamide plates. Resulting transformants were struck for single colony isolation on YNB+acetamide plates. Single colonies were selected, and the correct integration of the expression cassette is confirmed by PCR. Three independent transformants were tested in the fermentation condition described in TEST #5, and a representative isolate that demonstrated early fermentation rate and equivalent or higher final ethanol titer when compared to Strain 1 is designated Strain 1-26.

Strain 1-27: Saccharomyces cerevisiae Expressing a Modified Saccharomycopsis fibuligera Glucoamlase at Both Alleles of CYB2, and a Bacillus cereus Glyceraldehyde-3-Phosphate Dehydrogenase at Both Alleles of DLD1.

Strain 1-6 is co-transformed with SEQ ID NO: 32 and SEQ ID NO: 33, and the transformants were selected on ScD-Ura. Resulting transformants were struck for single colony isolation on ScD-Ura. Single colonies were selected, and the correct integration of the expression cassette is confirmed by PCR. Three independent transformants were moved forward for integration of the second copy of the expression cassette at the DLD1 locus.

Three independent sisters strains containing 1 copy of SEQ ID NO: 32 and SEQ ID NO: 33 were co-transformed with SEQ ID NO: 34 and SEQ ID NO: 35. Transformants were selected on YNB+acetamide plates. Resulting transformants were struck for single colony isolation on YNB+acetamide plates. Single colonies were selected, and the correct integration of the expression cassette is confirmed by PCR. Three independent transformants were tested in the fermentation condition described in TEST #5, and a representative isolate that demonstrated early fermentation rate and equivalent or higher final ethanol titer when compared to Strain 1 is designated Strain 1-27.

Strain 1-28: Saccharomyces cerevisiae Expressing a Modified Rhizopus delemar Glucoamlase at Both Alleles of FCY1, and a Bacillus cereus Glyceraldehyde-3-Phosphate Dehydrogenase at Both Alleles of DLD1.

Strain 1-14 is co-transformed with SEQ ID NO: 32 and SEQ ID NO: 33, and the transformants were selected on ScD-Ura. Resulting transformants were struck for single colony isolation on ScD-Ura. Single colonies were selected, and the correct integration of the expression cassette is confirmed by PCR. Three independent transformants were moved forward for integration of the second copy of the expression cassette at the DLD1 locus.

Three independent sisters strains containing 1 copy of SEQ ID NO: 32 and SEQ ID NO: 33 were co-transformed with SEQ ID NO: 34 and SEQ ID NO: 35. Transformants were selected on YNB+acetamide plates. Resulting transformants were struck for single colony isolation on YNB+acetamide plates. Single colonies were selected, and the correct integration of the expression cassette is confirmed by PCR. Three independent transformants were tested in the fermentation condition described in TEST #5, and a representative isolate that demonstrated early fermentation rate and equivalent or higher final ethanol titer when compared to Strain 1 is designated Strain 1-28.

Strain 1-29: Saccharomyces cerevisiae Expressing a Modified Rhizopus microsporus Glucoamlase at Both Alleles of FCY1, and a Bacillus cereus Glyceraldehyde-3-Phosphate Dehydrogenase at Both Alleles of DLD1.

Strain 1-18 is co-transformed with SEQ ID NO: 32 and SEQ ID NO: 33, and the transformants were selected on ScD-Ura. Resulting transformants were struck for single colony isolation on ScD-Ura. Single colonies were selected, and the correct integration of the expression cassette is confirmed by PCR. Three independent transformants were moved forward for integration of the second copy of the expression cassette at the DLD1 locus.

Three independent sisters strains containing 1 copy of SEQ ID NO: 32 and SEQ ID NO: 33 were co-transformed with SEQ ID NO: 34 and SEQ ID NO: 35. Transformants were selected on YNB+acetamide plates. Resulting transformants were struck for single colony isolation on YNB+acetamide plates. Single colonies were selected, and the correct integration of the expression cassette is confirmed by PCR. Three independent transformants were tested in the fermentation condition described in TEST #5, and a representative isolate that demonstrated early fermentation rate and equivalent or higher final ethanol titer when compared to Strain 1 is designated Strain 1-29.

Strain 1-30: Saccharomyces cerevisiae Expressing a Modified Rhizopus oryzae Glucoamlase at Both Alleles of CYB2, Bacillus cereus Glyceraldehyde-3-Phosphate Dehydrogenase at Both Alleles of GPP1, and One Copy of the Saccharomyces cerevisiae Trehalose-6-Phosphate Synthase and Trehalose-6-Phosphate Synthase/Phosphatase at One Allele of ADH2.

Strain 1-22 is co-transformed with SEQ ID NO: 36 and 37. SEQ ID NO: 36 contains the following elements: i) DNA homologous to the 5′ region of the native ADH2 gene; and ii) an expression cassette for the native Saccharomyces cerevisiae Trehalose-6-Phosphate Synthase (TPS1) (SEQ ID NO: 43), under control of the native Saccharomyces cerevisiae 3-Phosphoglycerate kinase (PGK1) promoter and the native Saccharomyces cerevisiae Vacuolar protein sorting (VPS13) terminator; and iii) the native Saccharomyces cerevisiae Triose-Phosphate Isomerase (TPI1) promoter and a portion of Kanamycin resistance (G418^{R}) marker. SEQ ID NO: 37 contains the following elements: i) a portion of the Kanamycin resistance (G418^{R}) marker and the native Saccharomyces cerevisiae alcohol dehydrogenase (ADH1) terminator; and ii) an expression cassette for the native Saccharomyces cerevisiae Trehalose-6-Phosphate Synthase/phosphatase (TPS2) (SEQ ID NO: 44), under control of the native Saccharomyces cerevisiae Triose-Phosphate dehydrogenase (TDH3) promoter and the native Saccharomyces cerevisiae Pheromone regulated membrane protein (PRM9) terminator; and iii) DNA homologous to the 3′ region of the native ADH2 gene. Transformants are selected on YPD+G418 media [1% Yeast extract, 2% Peptone, 2% Glucose, 2% Agar and 200 mg/L Geneticin selective antibiotic (G418 Sulfate)]. Resulting transformants are struck for single colony isolation on selection media. Single colonies were selected, and the correct integration of the expression cassette is confirmed by sequencing. Three independent transformants were tested in a shake flask fermentation and a representative isolate is designated Strain 1-30.

Strain 1-31: Saccharomyces cerevisiae Expressing a Modified Rhizopus oryzae Glucoamlase at Both Alleles of CYB2, Bacillus cereus Glyceraldehyde-3-Phosphate Dehydrogenase at Both Alleles of GPD1, and One Copy of the Saccharomyces cerevisiae Trehalose-6-Phosphate Synthase and Trehalose-6-Phosphate Synthase/Phosphatase at One Allele of ADH2.

Strain 1-20 is co-transformed with SEQ ID NO: 36 and 37, and transformants are selected on YPD+G418 media. Resulting transformants are struck for single colony isolation on selection media. Single colonies are selected, and the correct integration of the expression cassette is confirmed by sequencing. Three independent transformants are tested in a shake flask fermentation and a representative isolate is designated Strain 1-31.

TABLE 1
Description of sequences

| SEQ ID | Description |
|---|---|
| 1 | ARO4-OFP cassette; URA3 deletion |
| 2 | amdS cassette; URA3 deletion |
| 3 | Cre recombinase |
| 4 | 2u plasmid |
| 5 | Sf GA expression cassette; 5′ URA3 |
| 6 | Sf GA expression cassette; 3′ URA3 |
| 7 | Sf GA expression cassette; 5′ amdS |
| 8 | Sf GA expression cassette; 3′ amdS |
| 9 | Cre recombinase plasmid for marker loopout |
| 10 | URA3 repair cassette |
| 11 | Ro GA expression cassette; 5′ URA3 |
| 12 | Ro GA expression cassette; 3′ URA3 |
| 13 | Ro GA expression cassette; 5′ amdS |
| 14 | Ro GA expression cassette; 3′ amdS |
| 15 | Rdel GA expression cassette; 5′ URA3 |
| 16 | Rdel GA expression cassette; 3′ URA3 |
| 17 | Rdel GA expression cassette; 5′ amdS |
| 18 | Rdel GA expression cassette; 3′ amdS |
| 19 | Rmic GA expression cassette; 5′ URA3 |
| 20 | 3′ URA3 cassette @ fcy1 |
| 21 | Rmic GA expression cassette; 5′ amdS |
| 22 | 3′ amdS cassette@ fcy1 |
| 23 | Bc gapN expression cassette @ gpd1; 5′ URA3 |
| 24 | 3′ URA3 cassette @ gpd1 |
| 25 | Bc gapN expression cassette @ gpd1; 5′ amdS |
| 26 | 3′ amdS cassette @ gpd1 |
| 27 | gpp1 deletion cassette; K.lactis URA3; URA3+ |
| 28 | Bc gapN expression cassette @ gpp1; 5′ URA3 |
| 29 | 3′ URA3 cassette @ gpp1 |
| 30 | Bc gapN expression cassette @ gpp1; 5′ amdS |
| 31 | 3′ amdS cassette @ gpp1 |
| 32 | Bc gapN expression cassette @ dld1; 5′ URA3 |
| 33 | 3′ URA3 cassette @ dld1 |
| 34 | Bc gapN expression cassette @ dld1; 5′ amdS |
| 35 | 3′ amdS cassette @ dld1 |
| 36 | TPS1 expression cassette @ adh2; 5′ marker |
| 37 | TPS2 expression cassette @ adh2; 3′ marker |
| 38 | Sf GLA1 protein |
| 39 | Ro amyA protein |
| 40 | Rdel amyA protein |
| 41 | Rmic amyA protein |
| 42 | Bcereus gapN protein |
| 43 | Sc TPS1 protein |
| 44 | Sc TPS2 protein |
| 45 | Bcereus gapN DNA sequence |
| 46 | Sf GLA1 DNA sequence #1 |
| 47 | Sf GLA1 DNA sequence #2 |
| 48 | Sf GLA1 DNA sequence #3 |
| 49 | Sf GLA1 DNA sequence #4 |
| 50 | Ro amyA DNA sequence #1 |
| 51 | Ro amyA DNA sequence #2 |
| 52 | Rdel amyA DNA sequence #1 |
| 53 | Rdel amyA DNA sequence #2 |
| 54 | Rmic amyA DNA sequence |
| 55 | Sc TPS1 DNA sequence |
| 56 | Sc TPS2 DNA sequence |

TABLE 2
Description of Strains

| Strain | Parent | Description |
|---|---|---|
| Strain 1 | N/A | Saccharomyces cerevisiae (Lasaffre, Ethanol Red) |
| Strain 1-1 | Strain 1 | ura3Δ/URA3, ARO4-OFP+ |
| Strain 1-2 | Strain 1-1 | ura3Δ, ARO4-OFP+, amdS+ |
| Strain 1-3 | Strain 1-2 | ura3Δ |
| Strain 1-4 | Strain 1-3 | Saccharomycopsis fibuligera GLA1+; URA3+, |
| Strain 1-5 | Strain 1-4 | Saccharomycopsis fibuligera GLA1+; URA3+, amdS+ |
| Strain 1-6 | Strain 1-5 | Saccharomycopsis fibuligera GLA1+; ura3− |
| Strain 1-7 | Strain 1-6 | Saccharomycopsis fibuligera GLA1+; URA3+ |
| Strian 1-8 | Strain 1-3 | Rhizopus oryzae amyA+; URA3+, |
| Strain 1-9 | Strain 1-8 | Rhizopus oryzae amyA+; URA3+, amdS+ |
| Strain 1-10 | Strain 1-9 | Rhizopus oryzae amyA+; ura3− |
| Strain 1-11 | Strain 1-10 | Rhizopus oryzae amyA+; URA3+ |
| Strian 1-12 | Strain 1-3 | Rhizopus delemar amyA+; URA3+, |
| Strain 1-13 | Strian 1-12 | Rhizopus delemar amyA+; URA3+, amdS+ |
| Strain 1-14 | Strain 1-13 | Rhizopus delemar amyA+; ura3− |
| Strain 1-15 | Strain 1-14 | Rhizopus delemar amyA+; URA3+ |
| Strian 1-16 | Strain 1-3 | Rhizopus microsporus amyA+; URA3+, |
| Strain 1-17 | Strain 1-16 | Rhizopus microsporus amyA+; URA3+, amdS+ |
| Strain 1-18 | Strain 1-17 | Rhizopus microsporus amyA+; ura3− |
| Strain 1-19 | Strain 1-18 | Rhizopus microsporus amyA+; URA3+ |
| Strain 1-20 | Strain 1-10 | Rhizopus oryzae amyA+; Bacillus cereus gapN at GPD1 locus; URA3+, amdS+ |
| Strain 1-21 | Strain 1-10 | Rhizopus oryzae amyA+; Kluyveromyces lactis URA3 at GPP1 locus; URA3+ |
| Strain 1-22 | Strain 1-10 | Rhizopus oryzae amyA+; Bacillus cereus gapN at GPP1 locus; URA3+, amdS+ |
| Strain 1-23 | Strain 1-6 | Saccharomycopsis fibuligera GLA1+; Bacillus cereus gapN at GPP1 locus; URA3+, amdS+ |
| Strain 1-24 | Strain 1-14 | Rhizopus delemar amyA+; Bacillus cereus gapN at GPP1 locus; URA3+, amdS+ |
| Strain 1-25 | Strain 1-18 | Rhizopus microsporus amyA+; Bacillus cereus gapN at GPP1 locus; URA3+, amdS+ |
| Strain 1-26 | Strain 1-10 | Rhizopus oryzae amyA+; Bacillus cereus gapN at DLD1 locus; URA3+, amdS+ |
| Strain 1-27 | Strain 1-6 | Saccharomycopsis fibuligera GLA1+; Bacillus cereus gapN at DLD1 locus; URA3+, amdS+ |
| Strain 1-28 | Strain 1-14 | Rhizopus delemar amyA+; Bacillus cereus gapN at DLD1 locus; URA3+, amdS+ |
| Strain 1-29 | Strain 1-18 | Rhizopus microsporus amyA+; Bacillus cereus gapN at DLD1 locus; URA3+, amdS+ |
| Strain 1-30 | Strain 1-22 | Rhizopus oryzae amyA+; Bacillus cereus gapN at GPP1 locus; |
| | | Saccharomyces cerevisiae TPS1/2 at ADH2 locus; URA3+, amdS+, G418+ |
| Strain 1-31 | Strain 1-20 | Rhizopus oryzae amyA+; Bacillus cereus gapN at GPD1 locus; |
| | | Saccharomyces cerevisiae TPS1/2 at ADH2 locus; URA3+, amdS+, G418+ |

### Example 2. Effect of Gpp1 Deletion and Overexpression of the B. cereus gapN Gene at the GPP1 Locus in a Rhizopus oryzae (Ro) Glucoamylase Enabled Yeast Strain in Corn Mash

The impact of reducing expression of GPP1 and overexpressing GAPN on ethanol production was evaluated as described in Test #1. The GPP1 gene was deleted (Strains 1-21 and 1-22) and gapN was overexpressed (Strain 1-22) in strains of S. cerevisiae with enabled glucoamylase. Total Glucose Equivalents (TGE) was determined to be 279 g/kg glucose and that value was used to determine the yield differential between Strain 1-22 and the parent strain (Strain 1-11) as described in Test #3.

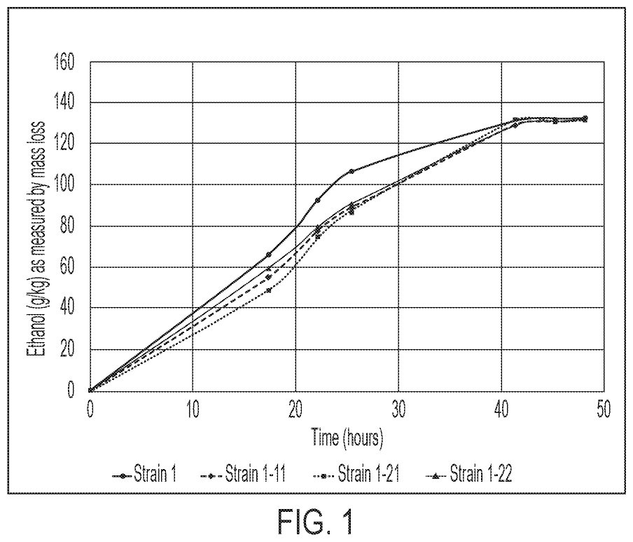

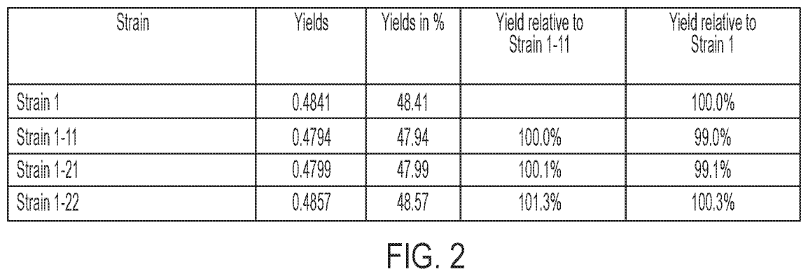

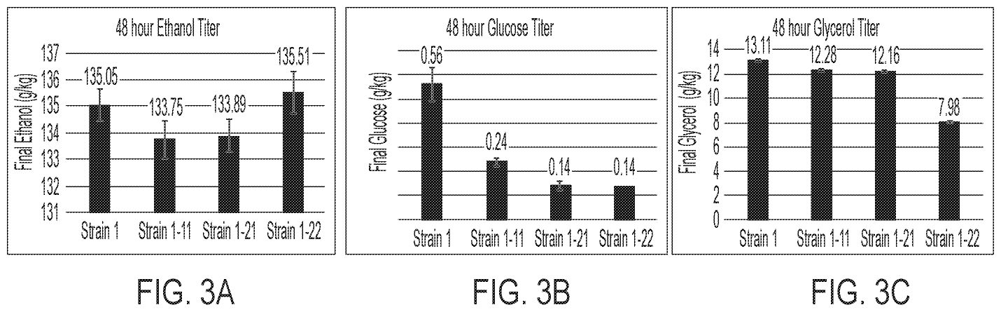

The results indicate that there was no impact on fermentation rate in the test strains (Strain 1-21 and 1-22) relative to the parent Strain 1-11 (FIG. 1) and that the residual glucose was <0.6 g/kg at 48 hours for all strains (FIG. 3B). The combination of gapN integrated at the GPP1 locus in the glucoamylase-enabled yeast strain (Strain 1-22) resulted a 4.3 g/L reduction in glycerol titer (FIG. 3C), a 1.8 g/L increase in ethanol titer (FIG. 3A) and a 1.3% higher yield compared to the parent (Strain 1-11) at 48 hours (FIG. 2).

### Example 3. Comparison of Overexpressing the B. cereus gapN Gene at the GPD1 Locus or GPP1 Locus in a Rhizopus oryzae (Ro) Glucoamylase Enabled Yeast Strain in Corn Mash

The impact of overexpressing the B. cereus gapN gene at the GPD1 locus (Strain 1-20) or GPP1 locus (Strain 1-22) in a Rhizopus oryzae (Ro) glucoamylase enabled yeast strain was compared in corn mash as described in Test #1. The test strains (Strains 1-20 and 1-22) were compared to parent strain (Strain 1-11) and a wild type strain (Strain 1).

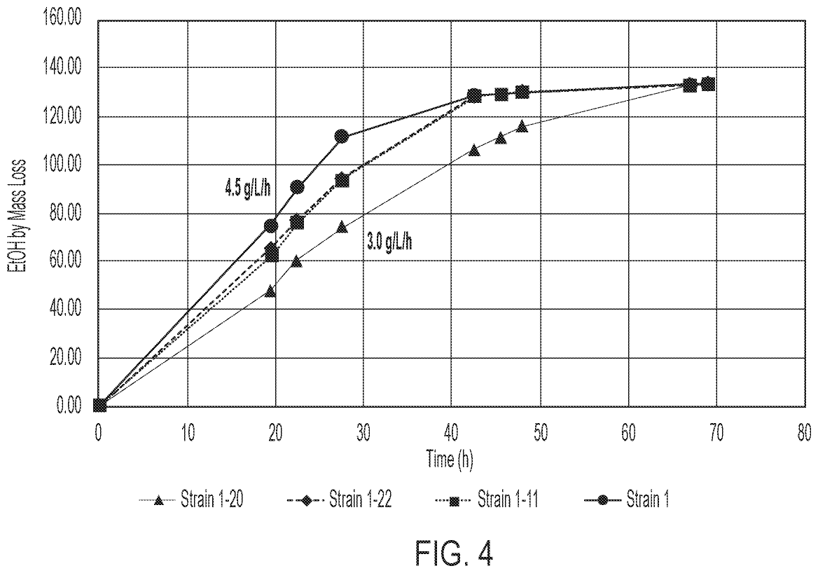

Strain 1-20 was found to produce 17% lower ethanol in 40 hrs in corn mash (calculated by mass loss), demonstrating a significant rate loss (FIG. 4). By contrast, addition of GAPN to the GPP1 locus (Strain 1-22) led to equivalent ethanol production as Strain 1 by 40 hrs (FIG. 4). At 48 hrs, average ethanol titer by mass loss (g/L) was as follows for each strain in FIG. 4: 115.62 g/L (Strain 1-20), 130.47 g/L (Strain 1-22), 130.09 g/L (Strain 1-11) and 130.16 g/L (Strain 1). These data indicate that the addition of GAPN at the GPD1 locus is less favorable as it results in an increased fermentation penalty relative to the addition of GAPN to a locus other than GPD1, such as to the locus GPP1.

### Example 4. Ethanol Production and Glycerol Reduction in Strains 1-21 and 1-22 in Light Steep Water Liquifact (Wet Milling Feedstock) Airlock Flasks

The effect of reducing expression of GPP1 and overexpressing GAPN on ethanol production in Steep Water Liquifact (wet milling feedstock) airlock flasks was tested using Strain 1, Strain 1-11, Strain 1-21, and Strain 1-22, measuring ethanol titer and glycerol levels as described in Test #4.

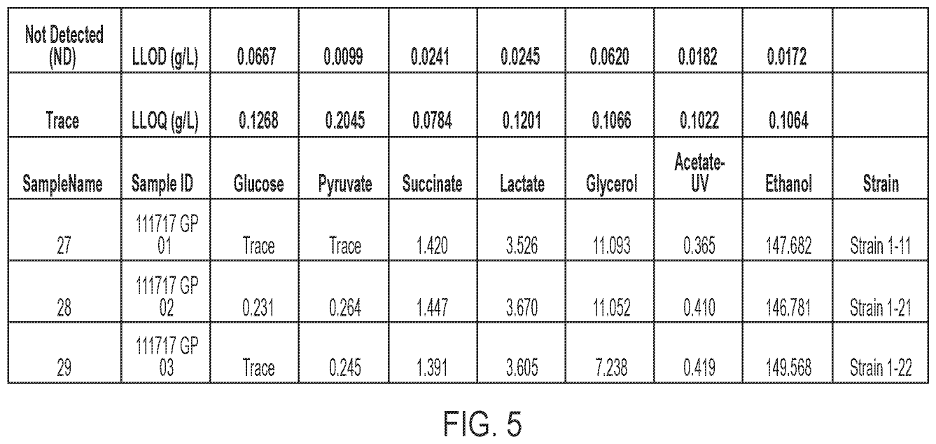

The data revealed a 3.9 g/L reduction in glycerol, and a 1.9 g/L increase in ethanol in Strain 1-22 compared to Strain 1-11 (FIG. 5). This is a similar glycerol titer reduction and ethanol titer increase to that observed in corn mash (dry grind ethanol feedstock). FIG. 5 shows the results in a Light Steep Water Liquifact LSW/LQ media (Wet Milling feedstock) at 72 hrs.

### Example 5: Comparison of Glucoamylase Backgrounds, and Evaluation of Strains Expressing Tps 1/2

A fermentation experiment (Test #1) (4 replicates per strain) was run comparing the effect of overexpressing the B. cereus gapN gene at the GPD1 locus (Strain 1-20) or GPP1 locus (Strain 1-22) in a Rhizopus oryzae (Ro) glucoamylase enabled yeast strain. Additionally, the Tps1/2 proteins were overexpressed in Strain 1-20 and 1-22 to evaluate whether these genes would improve the ethanol fermentation rate. The resulting strains, Strain 1-30 (gapN at the GPP1 locus) and Strain 1-31 (gapN at the GPD1 locus), both contain 1 overexpressed copy of the Tps1/2 genes at the ADH2 locus. The impact of the B. cereus gapN gene at the GPP1 locus was also evaluated in three different glucoamylase backgrounds RoGA (Strain 1-22), Rdel (Strain 1-24), and Rmic (Strain 1-25) in order to determine whether the glucoamylase gene source would impact ethanol production in corn mash. All strains were run to 48 hrs except for Strains 1-20 and 1-31 (containing the deletion of the GPD1 locus) which were run to 67 hrs.

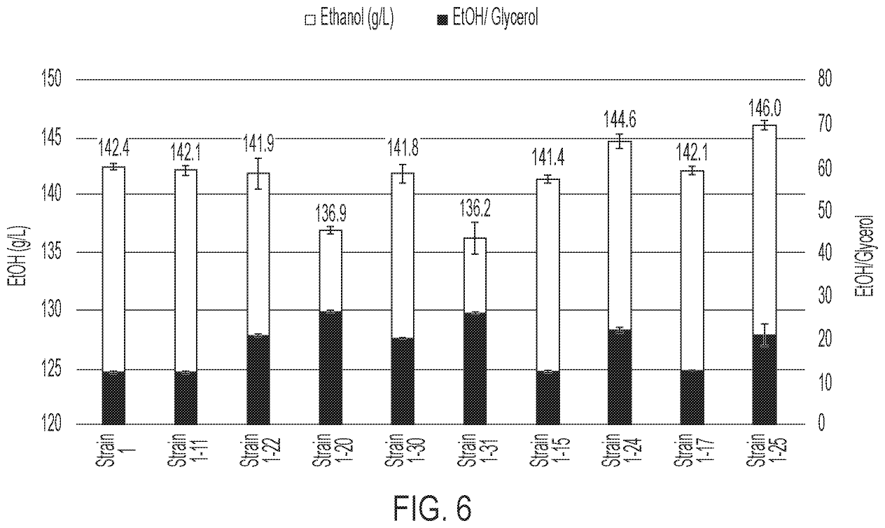

FIG. 6 is a graph showing that Strains 1-24 and 1-25 produced 2.2 g/L and 3.6 g/L higher ethanol titers, respectively, compared to Strain 1 in corn mash.

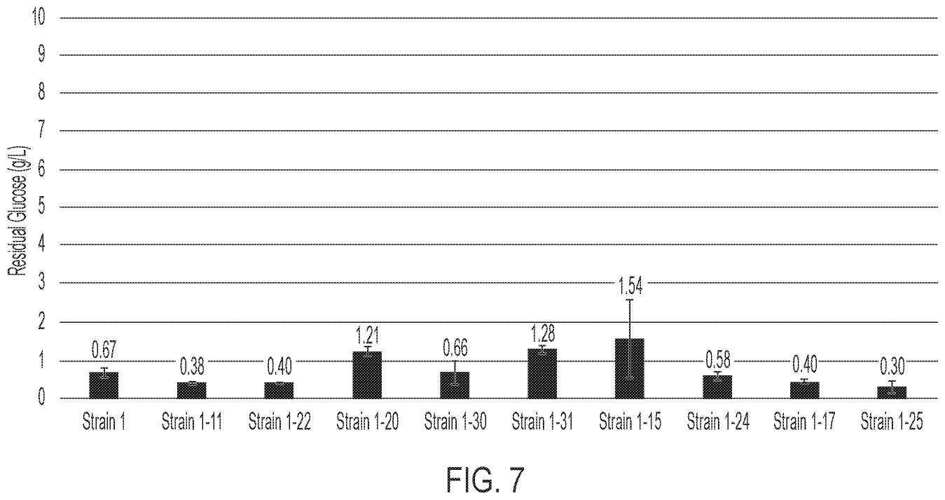

FIG. 7 is a graph showing residual glucose in Strains 1-24 and 1-25 relative to Strain 1. Strains containing the gapN gene at the GPP1 locus show residual glucose values of <1.5 g/kg at the end of fermentation.

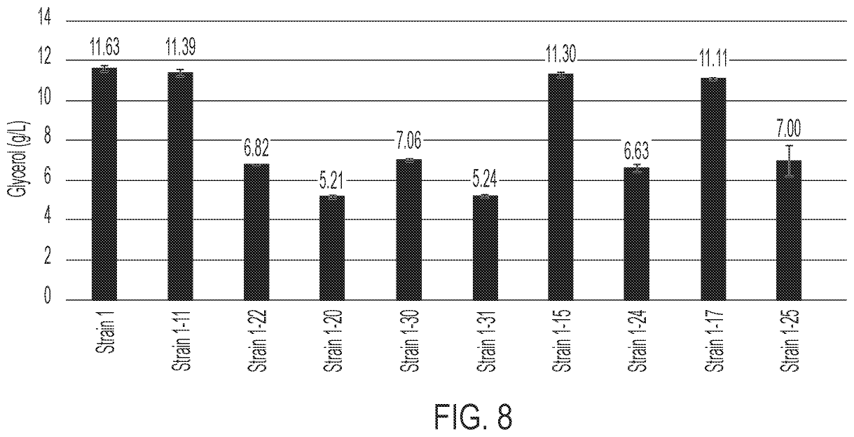

FIG. 8 is a graph showing that Strains 1-24 and 1-25 produced a 5.0 g/L and 4.6 g/L reduction, respectively, in glycerol titer relative to Strain 1 in corn mash.

Strains in which the B. cereus gapN gene was inserted at the GPD1 locus never reached the titers of the parent strain due to a fermentation burden. By contrast, strains in which the B. cereus gapN gene was inserted at the GPP1 locus performed better.

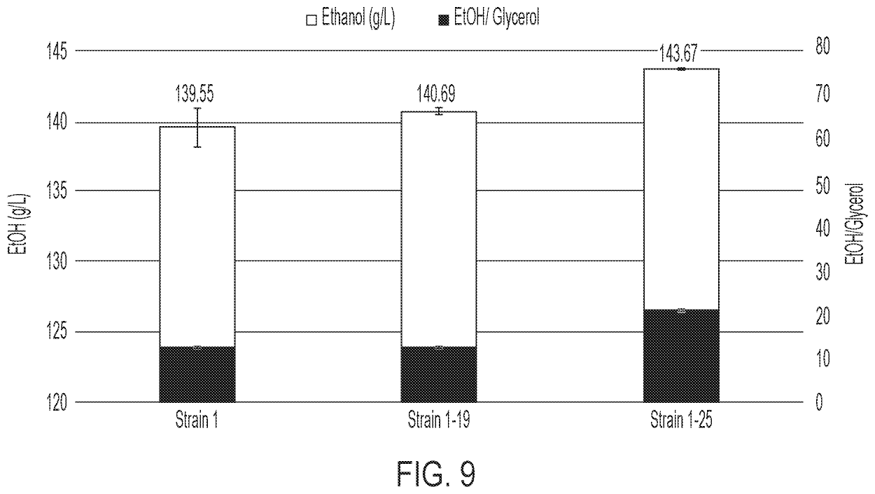

FIG. 9 shows that Strain 1-25 produces a 4.1 g/L increase in ethanol titer relative to Strain 1 in corn mash at 47 hrs.

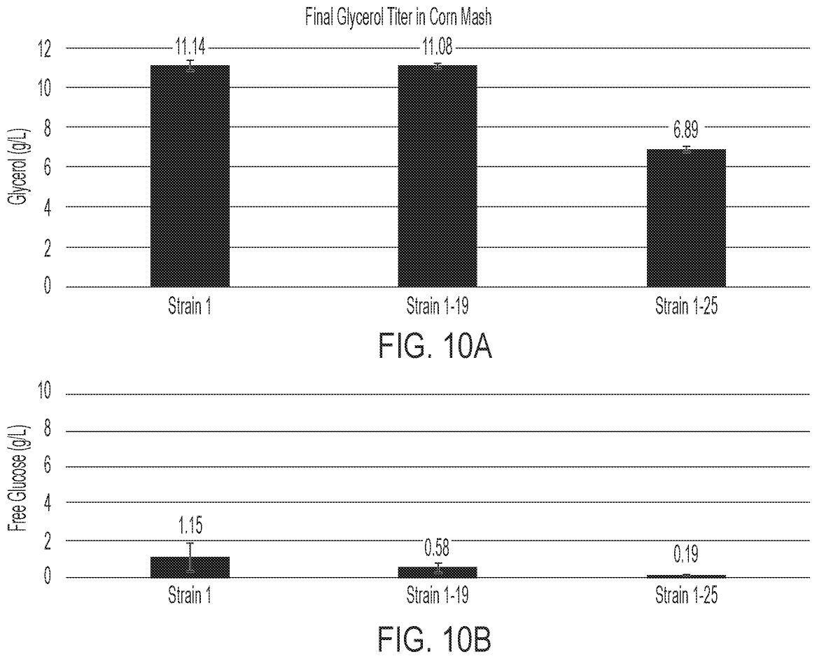

FIG. 10 shows that Strain 1-25 produces a 4.3 g/L reduction in glycerol titer relative to Strain 1 in corn mash. FIG. 10B shows residual glucose at the end of fermentation (47 hrs) in corn mash to be less than 1.5 g/L.

Strain 1-25 exhibits improved ethanol titer and decreased glycerol titer, without a negative impact on fermentative rate.

### Example 6. Comparison of Overexpressing the B. cereus gapN Gene at the GPP1 Locus or DLD1 Locus in a Variety of Glucoamylase Enabled Yeast Strains in Corn Mash

The impact of overexpressing the B. cereus gapN gene at the GPP1 locus (Strain 1-22, 1-23, 1-24, and 1-25) or DLD1 locus (Strain 1-27, 1-28, and 1-29) in a glucoamylase enabled yeast strain was compared in corn mash as described in Test #1. The test strains (Strain 1-22, 1-23, 1-24, 1-25, 1-27, 1-28, and 1-29) were compared to parent strains (Strain 1-7, 1-11, 1-15, and 1-19) and a wild type strain (Strain 1).

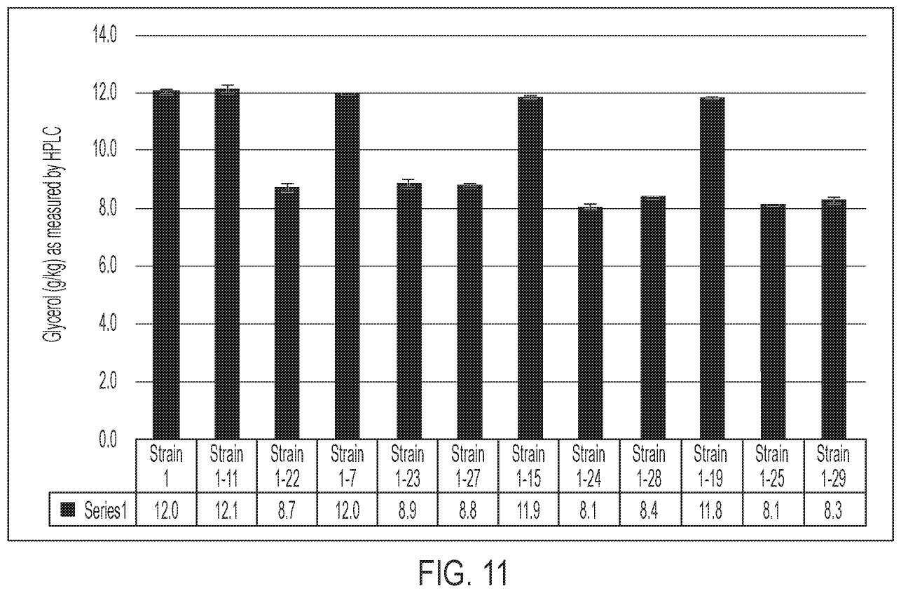

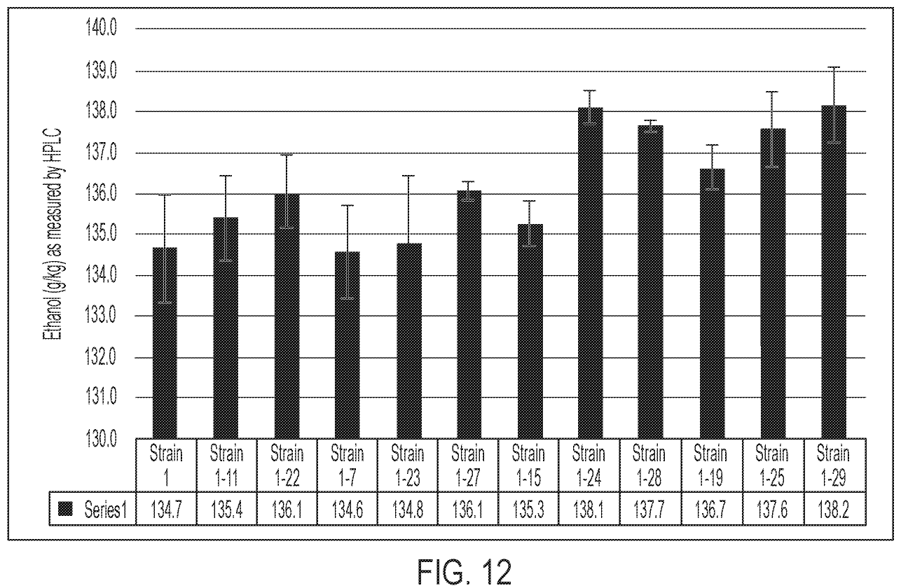

Addition of the B. cereus gapN to both the GPP1 locus and the DLD1 locus resulted in reducing the glycerol titer by between 3.1 g/kg and 3.9 g/kg depending on the glucoamylase background (FIG. 11). In general, strains that contained the gapN, regardless of the integration site, demonstrated ethanol titer increases over the respective parent strain and compared to the wild type strain (Strain 1) (FIG. 12). The ethanol titer increase was at least 1.4 g/kg in all strains except for Strain 1-23. While Strain 1-23 demonstrated a glycerol reduction of 3.1 g/kg compared to the parental control (Strain 1-7), the ethanol titers were similar. Strain 1-29 showed the highest increase in ethanol titer relative to Strain 1, with an increase of 3.5 g/kg (138.2 g/kg−134.7 g/kg).

These data indicate that the addition of GAPN at either the GPP1 locus or the DLD1 locus results in the increased ethanol titers at the end of fermentation as defined by Test #1.

### Example 7: Tests and Assays

### Test 1: Characterization of Strains in 33% DS Corn Mash at 33.3° C.

Strains were struck to a YPD plate and incubated at 30° C. until single colonies were visible (1-2 days). Cells from a YPD plate were scraped into pH 7.0 sterile phosphate buffer and the optical density (OD600) is measured. Optical density is measured at wavelength of 600 nm with a 1 cm path length using a model Genesys 20 Visible Spectrophotometer (Thermo Scientific). A shake flask is inoculated with the volume of the cell slurry necessary to reach an initial OD600 of 0.1. The inoculation volume is typically around 66 μl. Immediately prior to inoculating, the following materials were added to each 250 ml baffled shake flask: 50 grams of liquified corn mash, 1900 of 500 g/L filter-sterilized urea, and 2.50 of a 100 mg/ml filter sterilized stock of ampicillin. For the shake flasks containing the Ethanol Red® control strain (Strain 1), a quantity of glucoamylase (Spirizyme Fuel HS™ Novozymes; lot NAPM3771) to achieve a dose of 0.33 AGU/g of Dry Solids is added to the flasks, and 0.0825 AGU/g of Dry Solids (or a 25% of the dose provided to Ethanol Red®) of glucoamylase (Spirizyme Fuel HS™ Novozymes; lot NAPM3771) is added to the flasks containing the glucoamylase expressing yeast. Glucoamylase activity is measured using the Glucoamylase Activity Assay (described below). At least duplicate flasks for each strain were incubated at 33.3° C. with shaking in an orbital shaker at 100 rpm for approximately 48 hours. At 48 hours, 1 ml samples were taken and analyzed for ethanol and glucose concentrations in the broth by high performance liquid chromatography with refractive index detector.

### Test 2: Characterization of Strains in 33% DS Corn Mash at 33.3° C. (TEST #2)

Strains were struck to a YPD plate and incubated at 30° C. until single colonies were visible (1-2 days). Cells from a YPD plate were scraped into pH 7.0 sterile phosphate buffer and the optical density (OD600) is measured. Optical density is measured at a wavelength of 600 nm with a 1 cm path length using a model Genesys 20 Visible Spectrophotometer (Thermo Scientific). A shake flask is inoculated with the volume of the cell slurry necessary to reach an initial OD600 of 0.1. The inoculation volume is typically around 66 μl. Immediately prior to inoculating, the following materials were added to each 250 ml baffled shake flask: 50 grams of liquified corn mash, 1900 of 500 g/L filter-sterilized urea, and 2.50 of a 100 mg/ml filter sterilized stock of ampicillin. The shake flasks received a quantity of glucoamylase (Spirizyme Fuel HS™ Novozymes; lot NAPM3771) to achieve a dose of 0.33 AGU/g of Dry Solids. Glucamylase activity is measured using the Glucoamylase Activity Assay (defined below). At least duplicate flasks for each strain were incubated at 33.3° C. with shaking in an orbital shaker at 100 rpm for approximately 48 hours. At 48 hours, 1 ml samples were taken and analyzed for ethanol and glucose concentrations in the broth by high performance liquid chromatography with refractive index detector.

### Test 3: Yield Calculation

The equation for Ethanol Yield can be defined as: (Ethanol Titer at Time final−Ethanol Titer at Time zero) divided by TGE at Time zero.

$$Ethanol Yield ( \% ) = \frac{( Ethanol Titer at {T}_{final} - Ethanol Titer at {T}_{zero} )}{Total Glucose Equivalents at {T}_{zero}} \times 100$$

When calculating the yield difference between a glycerol reduction strain and a control strain, the ethanol yield of the control strain is subtracted from the ethanol yield of the glycerol reduction strain. For example, Strain 1-24 and Strain 1 were run in a corn mash fermentation as described in Test #1. The starting media was determined to have a TGE value of 280 g/kg glucose and there was 0 g/kg ethanol. At 48 hours the fermentation broth was measured by HPLC and it was determined that Strain 1-24 reached a final ethanol titer of 130 g/kg and Strain 1 reached a final ethanol titer of 128 g/kg. Based on the yield calculation above, it can be determined that Strain 1-24 had an ethanol yield of 46.4% (130 g/kg ethanol divided by 280 g/kg TGE) and Strain 1 had an ethanol yield of 45.7% (128 g/kg ethanol divided by 280 g/kg TGE). By using the ethanol yield of Strain 1-24 (46.4%) and subtracting the ethanol yield of Strain 1 (45.7%) it would be said that Strain 1-24 has a 0.7% higher ethanol yield than Strain 1.

Test 4: Evaluation of Genetically Modified Saccharomyces cerevisiae Strains in a Simultaneous Saccharification Fermentation (SSF) Shake Flask Assay

Strains were struck to a ScD-ura plate and incubated at 30° C. until single colonies were visible (2-3 days). Cells from the ScD-ura plate were scraped into sterile shake flask medium and the optical density (OD600) is measured. Optical density is measured at a wavelength of 600 nm with a 1 cm path length using a model Genesys 20 spectrophotometer (Thermo Scientific). A shake flask is inoculated with the cell slurry to reach an initial OD600 of 0.1. Immediately prior to inoculating, 50 mL of shake flask medium was added to a 250 mL baffled shake flask sealed with air-lock containing 4 mls of sterilized canola oil. The shake flask medium consisted of 725 g partially hydrolyzed corn starch, 150 g filtered sterilized (0.2 μm) light steep water, 10 g water, 25 g glucose, and 1 g urea. Strains were incubated at 30° C. with shaking in an orbital shake at 100 rpm for 72 hours. Samples were taken and analyzed for metabolite concentrations in the broth at the end of fermentation by HPLC.

### Glucoamylase Activity Assay

Glucoamylase activity (AGU) refers to the amount of enzyme that hydrolyzes 1 micromole of maltose per minute under the standard reaction conditions. The following stock solutions were prepared: i) 10× stock solution of maltose (232 mM); and ii) a 2× stock of Na-acetate buffer pH 4.3 (200 mM). A 1:10 dilution of the glucoamylase stock was used as the starting material and diluted from there (0.899 g water+0.140 g glucoamylase=1.0139 g total). Serial dilutions (1:1) were made in water, with a total of six dilutions in the series, starting with the original 1:10 dilution.

In a 200 μl reaction volume, the following components were added in order: 100 μl of Na-acetate buffer pH 4.3, 20 μl of a 10× maltose stock solution (or water in the blank control), and 70 μl water. The reaction was prewarmed to 37° C. prior to adding 10 μl of the diluted enzyme solutions. After 5 minutes at 37° C., the reaction was quenched with 15 μl of concentrated H2504. Glucose concentration was determined using HPLC, and the activity of the enzyme was determined using the following calculation:
1. The concentration of glucose (grams/Liter) at the end of the reaction was divided by the Molecular Weight of glucose (180.156 grams/mole) to obtain a Molar concentration (mole/Liter) of glucose.
2. The Molar concentration was multiplied by the total volume of the reaction (215 μl), to obtain the micromole concentration of glucose.
3. The micromoles of Glucose calculated in Step Two (above) was divided by 2 to account for maltose serving as the substrate in the reaction (2 Glucose=1 Maltose). This number was divided by the grams of enzyme used in the assay itself. The lowest dilution was made as described above, 0.140 g in 1.1039 g water, then multiplying this dilution by the assay dilution (10 μl of enzyme divided by 215 μl total volume).
   For example, a reaction containing the components listed above returned a HPLC glucose concentration of 4.2 grams per liter, and the activity of the enzyme was determined to be 312.7. AGU/g.

TABLE 3
Example of amylase activity assay

| | | | | | Micromoles |
|---|---|---|---|---|---|
| Grams of glucose | Moles per liter | Moles per liter | Micromoles | | maltose per |
| per liter released | glucose released | maltose released | maltose per | Grams of | minute per gram |
| per minute | per minute | per minute | minute in assay | enzyme used | enzyme |
| 0.8414 | 0.0047 | 0.0023 | 0.5021 | 0.0016 | 312.7 |
| measured by HPLC | (0.8414/180.156) | (.0047/2) | (.0023*0.000215*1000000) | measured by scale | (.5021/.0016) |

### Test 5: Characterization of Strains in 33% DS Corn Mash at 33.3° C. in 50 ml Conical Tubes

Strains were struck to a YPD plate and incubated at 30° C. until single colonies were visible (1-2 days). Cells from a YPD plate were scraped into pH 7.0 sterile phosphate buffer and the optical density (OD600) was measured. Optical density was measured at a wavelength of 600 nm with a 1 cm path length using a model Genesys 20 Visible Spectrophotometer (Thermo Scientific). A 50 ml conical tube fitted with a 0.2 μm filter (Nalgene syringe filter, Thermo Scientific; catalog number: 727-2020) was inoculated with the volume of the cell slurry necessary to reach an initial OD600 of 0.1. The inoculation volume was typically around 26 μl. Immediately prior to inoculating, the following materials were added to each 50 ml conical tube (Fisher Scientific; catalog number: 05-539-13): 20 grams of liquified corn mash, 76 μl of 500 g/L filter-sterilized urea, and 1 μl of a 100 mg/ml filter sterilized stock of ampicillin. For the shake flasks containing the Ethanol Red® control strain, a quantity of glucoamylase (Spirizyme Fuel HS™ Novozymes; lot NAPM3771) to achieve a dose of 0.33 AGU/g of Dry Solids was added to the flasks, and 0.0825 AGU/g of Dry Solids (or a 25% of the dose provided to Ethanol Red®) of glucoamylase (Spirizyme Fuel HS™ Novozymes) was added to the flasks containing the glucoamylase expressing yeast. Glucoamylase activity was measured using the Glucoamylase Activity Assay (described above). Duplicate flasks for each strain were incubated at 33.3° C. with shaking in an orbital shaker at 100 rpm for approximately 48 hours. At 48 hours, 1 ml samples were taken and analyzed for ethanol and glucose concentrations in the broth by high performance liquid chromatography with refractive index detector.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety, particularly for the disclosure referenced herein.

What is claimed is:

1. An engineered yeast comprising: a recombinant nucleic acid encoding a glyceraldehyde-3-phosphate dehydrogenase (E.C. 1.2.1.9); reduced or eliminated expression of a gene encoding a glycerol-3-phosphate phosphatase (E.C. 3.1.3.21); and a recombinant nucleic acid encoding a glucoamylase, wherein the yeast is capable of producing at least 100 g/kg of ethanol and producing less than 1.5 g/kg residual glucose in 48 hours under Test 1 conditions.

2. The engineered yeast of claim 1, wherein the yeast is a post-whole-genome duplication yeast species.

3. The engineered yeast of claim 2, wherein the yeast is Saccharomyces cerevisiae.

4. The engineered yeast of any one of claims 1-3, wherein the engineered yeast produces an ethanol yield that is at least 0.5% higher than a control strain.

5. The engineered yeast of any one of claims 1-4, wherein the engineered yeast produces 30% less glycerol, 40% less glycerol, or 50% less glycerol than a control strain.

6. The engineered yeast of claim 5, wherein glycerol production is determined by Test 4.

7. The engineered yeast of any one of claims 1-6, wherein the glucoamylase (GA) has at least 80%, at least 85%, at least 90%, or at least 95% sequence identity to SEQ ID NO:38 (Saccharomycopsis fibuligera GA).

8. The engineered yeast of any one of claims 1-6, wherein the glucoamylase (GA) has at least 80%, at least 85%, at least 90%, or at least 95% sequence identity to SEQ ID NO:39 (Rhizopus oryzae amyA).

9. The engineered yeast of any one of claims 1-6, wherein the glucoamylase (GA) has at least 80%, at least 85%, at least 90%, or at least 95% sequence identity to SEQ ID NO:41 (Rhizopus microsporus GA).

10. The engineered yeast of any one of claims 1-6, wherein the glucoamylase (GA) has at least 80%, at least 85%, at least 90%, or at least 95% sequence identity to SEQ ID NO:40 (Rhizopus delemar GA).

11. An engineered Saccharomyces cerevisiae yeast comprising: a recombinant nucleic acid encoding a glyceraldehyde-3-phosphate dehydrogenase (E.C. 1.2.1.9); and reduced or eliminated expression of a gene encoding a glycerol-3-phosphate phosphatase (E.C. 3.1.3.21), wherein the yeast is capable of producing at least 100 g/kg of ethanol and producing less than 1.5 g/kg residual glucose in 48 hours under Test 2 conditions.

12. The engineered Saccharomyces cerevisiae yeast of claim 11, wherein the engineered yeast produces an ethanol yield that is at least 0.5% higher than a control strain.

13. The engineered Saccharomyces cerevisiae yeast of claim 11 or 12, wherein the engineered yeast produces 30% less glycerol, 40% less glycerol, or 50% less glycerol than a control strain.

14. The engineered yeast of claim 13, wherein glycerol production is determined by Test 4.

15. The engineered yeast of any one of claims 11-14, wherein the glucoamylase (GA) has at least 80%, at least 85%, at least 90%, or at least 95% sequence identity to SEQ ID NO:38 (Saccharomycopsis fibuligera GA).

16. The engineered yeast of any one of claims 11-14, wherein the glucoamylase (GA) has at least 80%, at least 85%, at least 90%, or at least 95% sequence identity to SEQ ID NO:39 (Rhizopus oryzae amyA).

17. The engineered yeast of any one of claims 11-14, wherein the glucoamylase (GA) has at least 80%, at least 85%, at least 90%, or at least 95% sequence identity to SEQ ID NO:41 (Rhizopus microsporus GA).

18. The engineered yeast of any one of claims 11-14, wherein the glucoamylase (GA) has at least 80%, at least 85%, at least 90%, or at least 95% sequence identity to SEQ ID NO:40 (Rhizopus delemar GA).

19. An engineered yeast comprising an exogenous nucleic acid encoding a glyceraldehyde-3-phosphate dehydrogenase (E.C. 1.2.1.9), and an exogenous nucleic acid encoding a glucoamylase (GA) having 80% or greater identity to SEQ ID NO:38 (Saccharomycopsis fibuligera GA), SEQ ID NO:41 (Rhizopus microsporus GA), SEQ ID NO:40 (Rhizopus delemar GA), or SEQ ID NO:39 (Rhizopus oryzae amyA), wherein the yeast is capable of producing at least 100 g/kg of ethanol and having less than 1.5 g/kg residual glucose in 48 hours under Test 1 conditions.

20. The engineered yeast of claim 19, wherein the yeast is a post-whole-genome duplication yeast species.

21. The engineered yeast of claim 20, wherein the yeast is Saccharomyces cerevisiae.

22. The engineered yeast of any one of claims 19-21, wherein the engineered yeast produces an ethanol yield that is at least 0.5% higher than a control strain.

23. The engineered yeast of any one of claims 19-22, wherein the engineered yeast produces 30% less glycerol, 40% less glycerol, or 50% less glycerol than a control strain.

24. The engineered yeast of claim 23, wherein glycerol production is determined by Test 4.

25. The engineered yeast of any one of claims 1-24, wherein the nucleic acid encoding a glyceraldehyde-3-phosphate dehydrogenase (E.C. 1.2.1.9) has at least 80%, at least 85%, at least 90%, or at least 95% sequence identity to SEQ ID NO: 45.

26. The engineered yeast of any one of claims 1-24, wherein the nucleic acid encoding a glyceraldehyde-3-phosphate dehydrogenase (E.C. 1.2.1.9) encodes a protein that has at least 80%, at least 85%, at least 90%, or at least 95% sequence identity to SEQ ID NO: 42.

27. The engineered yeast of any one of claims 1-26, wherein the engineered yeast comprises a nucleic acid having at least 80%, at least 85%, at least 90%, or at least 95% sequence identity to SEQ ID NO: 59.

28. The engineered yeast of any one of claims 19-24, wherein the engineered yeast has reduced or eliminated expression of a gene encoding a glycerol-3-phosphate phosphatase (E.C. 3.1.3.21).

29. The engineered yeast of any one of claims 1-28, wherein the engineered yeast has reduced or eliminated expression of a glycerol-3-phosphate dehydrogenase (E.C. 1.1.1.8).

30. The engineered yeast of any one of claims 1-29, wherein the engineered yeast is Saccharomyces cerevisiae and wherein the engineered yeast has reduced or eliminated expression of GPP1.

31. The engineered yeast of any one of claims 1-30, wherein the engineered yeast is Saccharomyces cerevisiae and wherein the engineered yeast has reduced or eliminated expression of GPP2.

32. The engineered yeast of any one of claims 29-31, wherein the engineered yeast is Saccharomyces cerevisiae and wherein the engineered yeast has reduced or eliminated expression of GPD1.

33. The engineered yeast of any one of claims 29-32, wherein the engineered yeast is Saccharomyces cerevisiae and wherein the engineered yeast has reduced or eliminated expression of GPD2.

34. The engineered yeast of any one of claims 29-32, wherein the engineered yeast is Saccharomyces cerevisiae and wherein the engineered yeast has reduced or eliminated expression of GPP1, GPP2, GPD1, or GPD2.

35. The engineered yeast of any one of claims 1-34, further comprising a nucleic acid encoding a trehalose-6-phosphate synthase (Tps1; E.C. 2.4.1.15).

36. The engineered yeast of claim 35, wherein the nucleic acid encoding a trehalose-6-phosphate synthase (Tps1; E.C. 2.4.1.15) has at least 80%, at least 85%, at least 90%, or at least 95% sequence identity to SEQ ID NO: 55.

37. The engineered yeast of claim 35, wherein the nucleic acid encoding a trehalose-6-phosphate synthase (Tps1; E.C. 2.4.1.15) encodes a protein that has at least 80%, at least 85%, at least 90%, or at least 95% sequence identity to SEQ ID NO: 43.

38. The engineered yeast of any one of claims 1-37, further comprising a nucleic acid encoding a trehalose-6-phosphate synthase (Tps2; EC 3.1.3.12).

39. The engineered yeast of claim 38, wherein the nucleic acid encoding a trehalose-6-phosphate synthase (Tps2; EC 3.1.3.12) has at least 80%, at least 85%, at least 90%, or at least 95% sequence identity to SEQ ID NO: 56.

40. The engineered yeast of claim 38, wherein the nucleic acid encoding a trehalose-6-phosphate synthase (Tps2; EC 3.1.3.12) encodes a protein that has at least 80%, at least 85%, at least 90%, or at least 95% sequence identity to SEQ ID NO: 44.

41. A method for producing ethanol comprising fermenting the yeast of any one of claims 1-40 with a fermentation substrate.

42. The method of claim 41, wherein the fermentation substrate comprises starch.

43. The method of claim 41, wherein the fermentation substrate comprises glucose.

44. The method of claim 41, wherein the fermentation substrate comprises sucrose.

45. The method of claim 42, wherein the starch is obtained from corn, wheat and/or cassava.

46. The method of any one of claims 41-45, wherein the method includes supplementation with glucoamylase.

47. A method for producing trehalose comprising fermenting the yeast of any one of claims 35-40 with a fermentation substrate.
